(19) United States
(12) Patent Application Publication
(10) Pub. No.: US 2023/0322899 A1
(43) Pub. Date: Oct. 12, 2023

(54) FUSION PROTEIN ENHANCING CELL THERAPY

(71) Applicants: INNOVATIVE CELLULAR THERAPEUTICS HOLDINGS, LTD., Grand Cayman (KY); Innovative Cellular Therapeutics Inc., Rockville, MD (US)

(72) Inventors: Chengfei Pu, Shanghai (CN); Xiaogang Shen, Shanghai (CN); Zhiyuan Cao, Shanghai (CN); Lei Xiao, Rockville, MD (US); Beibei Jia, Shanghai (CN); Le Tian, Rockville, MD (US)

(73) Assignees: Innovative Cellular Therapeutics Holdings, Ltd., Rockville, MD (US); Innovative Cellular Therapeutics, Inc., Rockville, MD (US)

(21) Appl. No.: 18/043,021

(22) PCT Filed: Aug. 24, 2021
(86) PCT No.: PCT/US2021/047330

§ 371 Date: Feb. 25, 2023

Related U.S. Application Data
(60) Provisional application No. 63/132,057, filed on Dec. 30, 2020.
(60) Provisional application No. 63/081,675, filed on Sep. 22, 2020.
(60) Provisional application No. 63/076,707, filed on Sep. 10, 2020.
(60) Provisional application No. 63/071,785, filed on Aug. 28, 2020.

(51) Int. Cl. C07K 14/705; C07K 14/725; A61K 38/21

(52) U.S. Cl. CPC C07K 14/70578; C07K 14/70521; C07K 14/70503; C07K 2319/03; A61K 38/217; C07K 2319/02; C07K 14/7051

(57) ABSTRACT

The present disclosure relates to a fusion protein and uses thereof. For example, the fusion protein comprises an extra-cellular domain, a transmembrane domain, and an intracellular domain. The extracellular domain is derived from a first molecule, and the intracellular domain is derived from a second molecule. The first molecule is different from the second molecule, and the second molecule comprises OX40, CD40, 4-1 BB, GITR, ICOS, CD28, CD27, HER2, EGFR, EL-10R, IL-12R, IL-18R1, IL-23R, GP130, or IL-15Ra.

### CROSS REFERENCE TO RELATED APPLICATIONS

This application claims the benefit of Provisional Application No. 63/132,057, filed Dec. 30, 2020; U.S. Provisional Application No. 63/081,675, filed Sep. 22, 2020; U.S. Provisional Application No. 63/076,707, filed Sep. 10, 2020; and U.S. Provisional Application No. 63/071,785, filed Aug. 28, 2020, which are incorporated herein by reference in their entirety.

### SEQUENCE LISTING INFORMATION

A computer readable textfile, entitled “ST25.txt,” created on or about Jul. 26, 2021, with a file size of about 333 KB, contains the sequence listing this application and is hereby incorporated by reference in its entirety.

### BACKGROUND

T cell therapy has achieved good clinical efficacy in cancer, such as B-cell acute lymphoblastic leukemia (B-ALL), chronic lymphocytic leukemia (CLL), and lymphoma. However, progress is relatively slow for the treatment of solid tumors. There is a need to further modify T cells to treat solid tumors.

### SUMMARY

Embodiments relate to a method of enhancing T cell response. The method includes introducing a polynucleotide encoding a chimeric antigen receptor (CAR) and a polynucleotide encoding an extracellular domain of CD40 into T cells to obtain modified T cells; contacting the modified T cells with an antigen that the CAR binds and a CD40 activator, thereby inducing T cell response, wherein the T cell response is greater than the T cell response induced by contacting the modified T cells with the antigen in the absence of a CD40 activator.

Embodiments relate to a modified cell comprising a CAR comprising an extracellular domain, a transmembrane domain, and an intracellular domain. The modified cell can further comprise a modified signal regulatory protein α (SIRPα) that interferes with an interaction between SIRPα and CD47. In embodiments, the modified SIRPα lacks a functional SIRPα intracellular domain.

Embodiments relate to a modified cell comprising a CAR comprising an extracellular domain, a transmembrane domain, and an intracellular domain, and a dominant negative form of SIGLEC-10.

This Summary is not intended to identify key features or essential features of the claimed subject matter, nor is it intended to be used to limit the scope of the claimed subject matter.

### BRIEF DESCRIPTION OF THE DRAWINGS

The Detailed Description is described with reference to the accompanying figures. The use of the same reference numbers in different figures indicates similar or identical items.

FIG. 1 is a schematic diagram of an exemplary CAR molecule and a fusion protein.

FIG. 2 shows CAR expression ratio and cell phenotypes.

FIG. 3 shows a schematic diagram of various vectors introduced into T cells.

FIG. 4 shows results of flow cytometry analysis of the expression of CD137 on CD4 positive T cells.

FIG. 5 shows results of flow cytometry analysis of the expression of CD137 on CD8 positive T cells.

FIG. 6 shows results of flow cytometry analysis of the overexpression of CD40 on CD4 positive T cells.

FIG. 7 shows results of flow cytometry analysis of the overexpression of CD40 on CD8 positive T cells.

FIG. 8 shows the overexpression of CD40 on CD4 positive T cells.

FIG. 9 shows the overexpression of CD40 on CD8 positive T cells.

FIG. 10 shows results of flow cytometry analysis of T cells including vectors encoding CD40 that down-regulated CD40 expression after receiving CD40L stimulation as compared to the T cells without CD40L stimulation.

FIG. 11 shows results of flow cytometry analysis of the overexpression of CD40 on T cells that down-regulated PD-1 expression after receiving CD40L stimulation.

FIG. 12 shows embodiments of various modified cells overexpressing co-stimulation molecules.

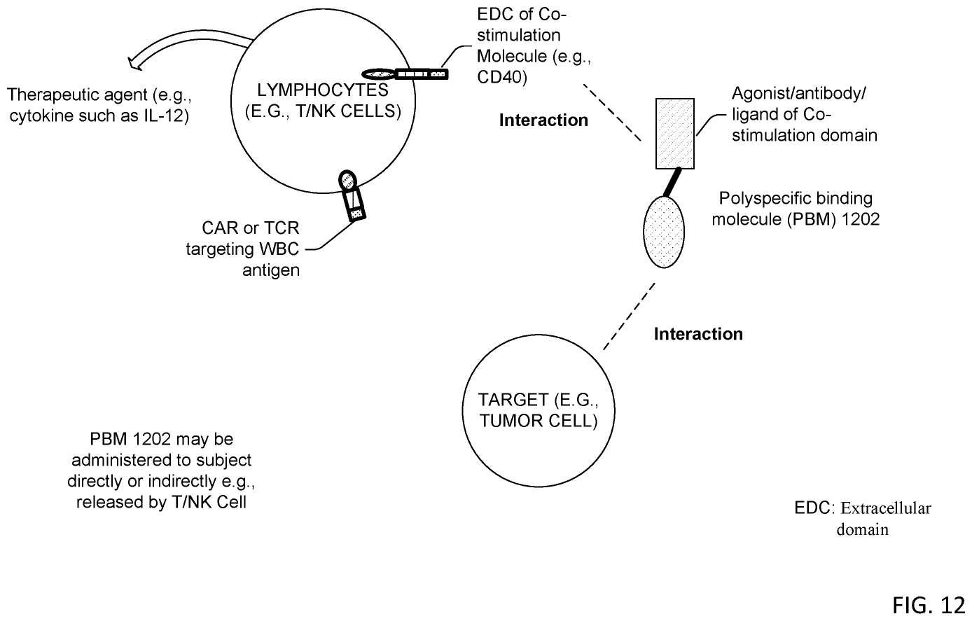

FIG. 13 shows embodiments of various modified cells overexpressing co-stimulation molecules.

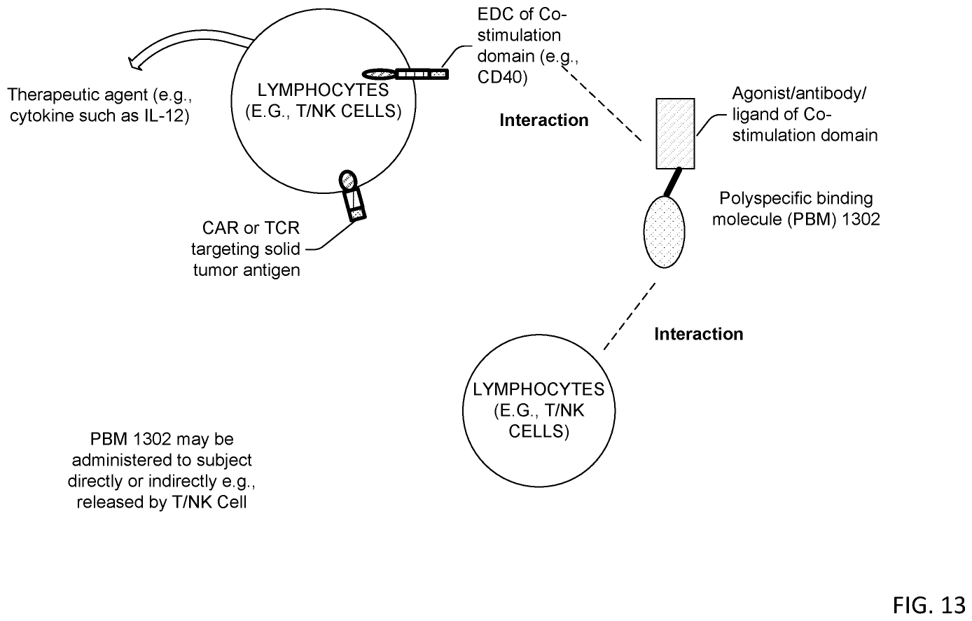

FIGS. 14 shows embodiments of various modified cells overexpressing co-stimulation molecules.

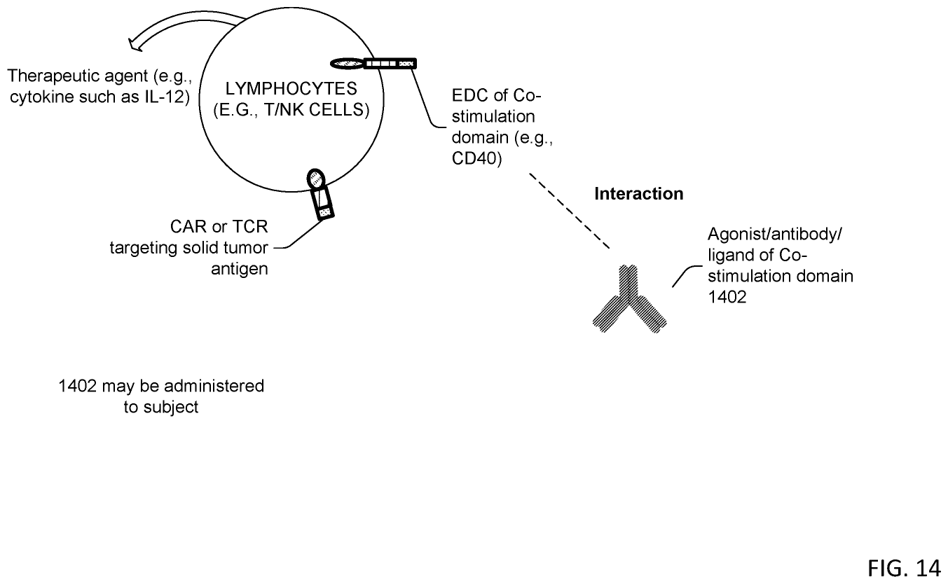

FIG. 15 shows a schematic diagram showing an example of a modified lymphocyte having less immunosuppression induced by CD47 as compared to a wide type lymphocyte.

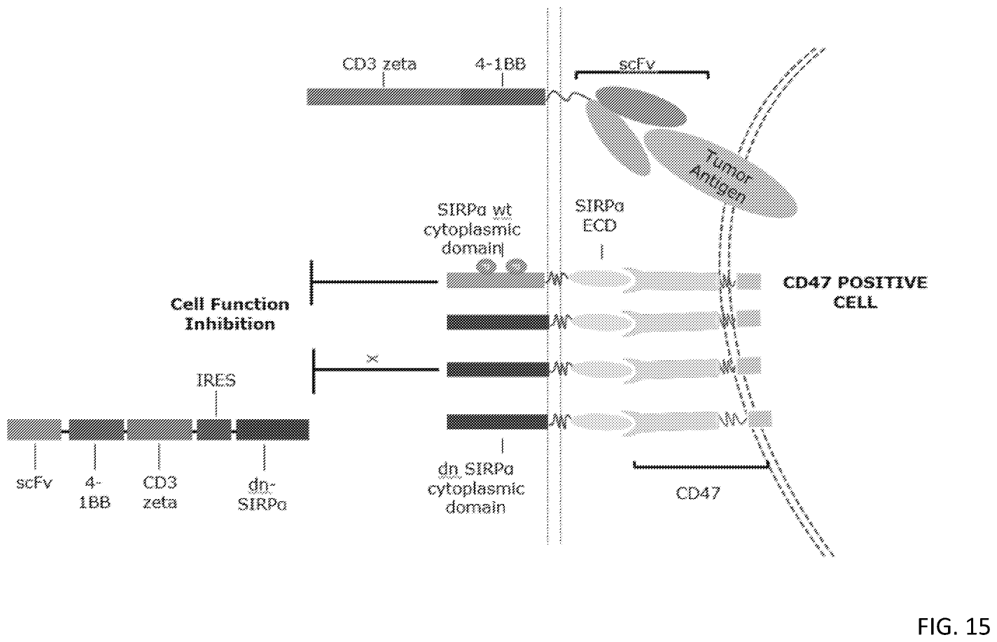

FIG. 16 shows a schematic diagram showing an example of a modified lymphocyte expressing one or more therapeutic agents.

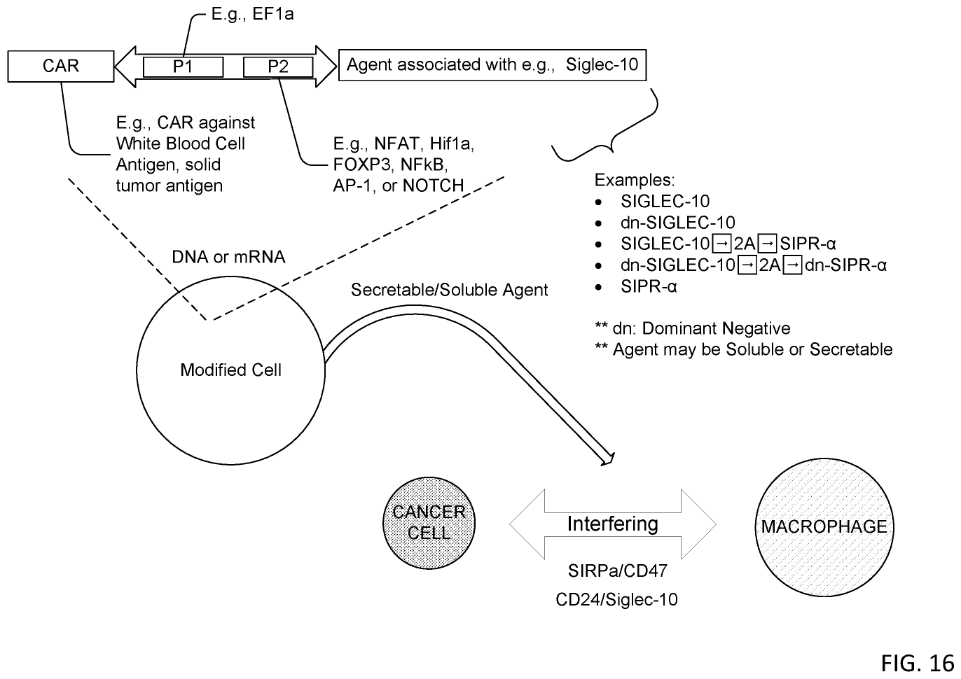

FIG. 17 shows a schematic diagram illustrating embodiments of immunotherapy using HRE.

FIG. 18 shows schematic structures of a polyspecific binding molecule targeting automiinue disease (AD) related T cell.

FIG. 19 shows schematic structures of a polyspecific binding molecule targeting AD related B cell.

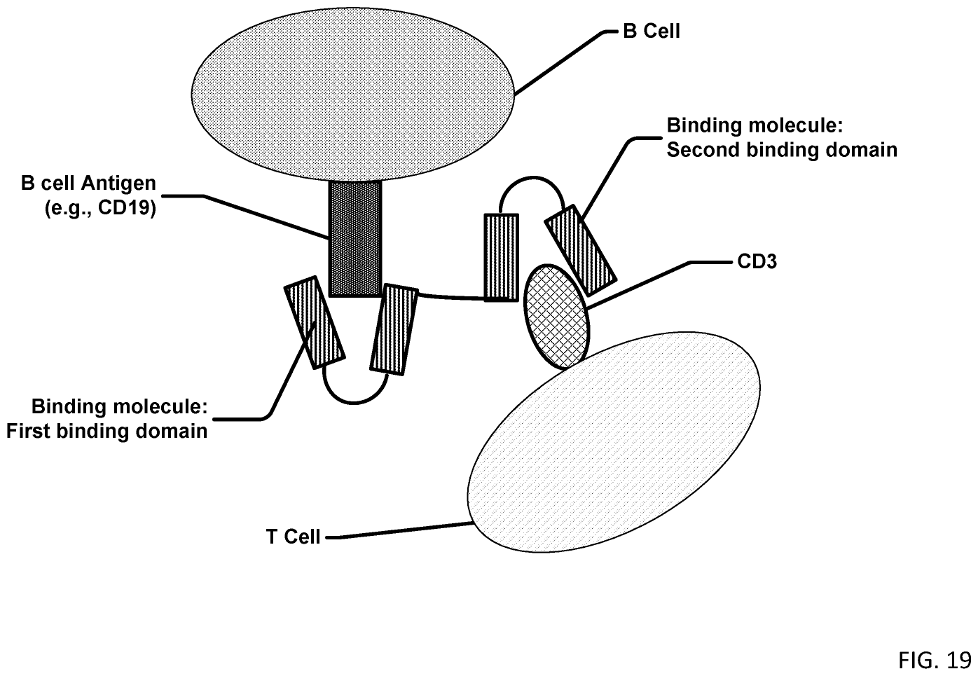

FIG. 20 shows relationship between expression of CLDN18.2 and GUCY2C in advanced gastric cancer patients.

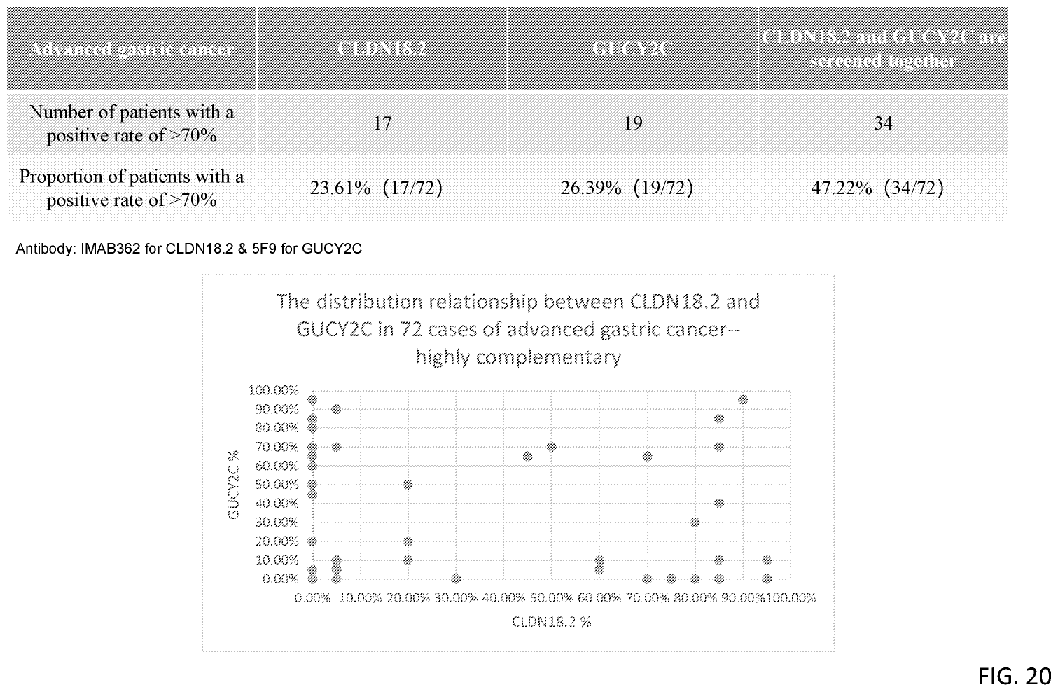

FIG. 21 shows immunochemistry staining of CLDN18.2 and GUCY2C in samples from advanced gastric cancer patients.

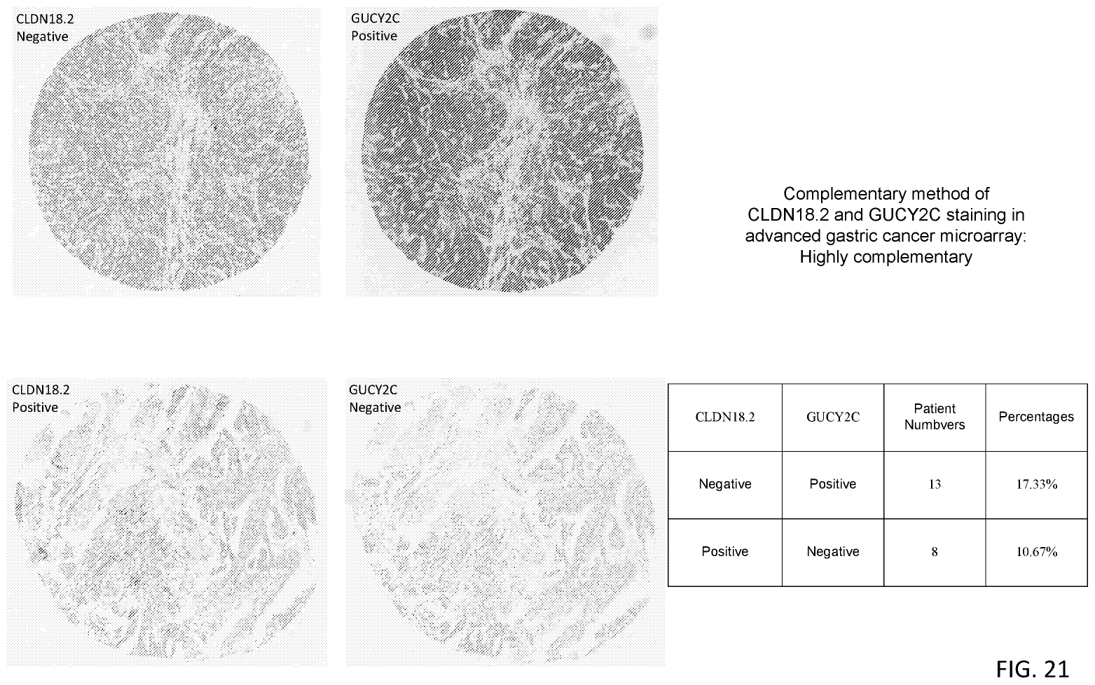

FIG. 22 shows immunochemistry staining of CLDN18.2 and GUCY2C in samples from advanced gastric cancer patients. The staining of CLDN18.2 and GUCY2C is complementary and overlapping.

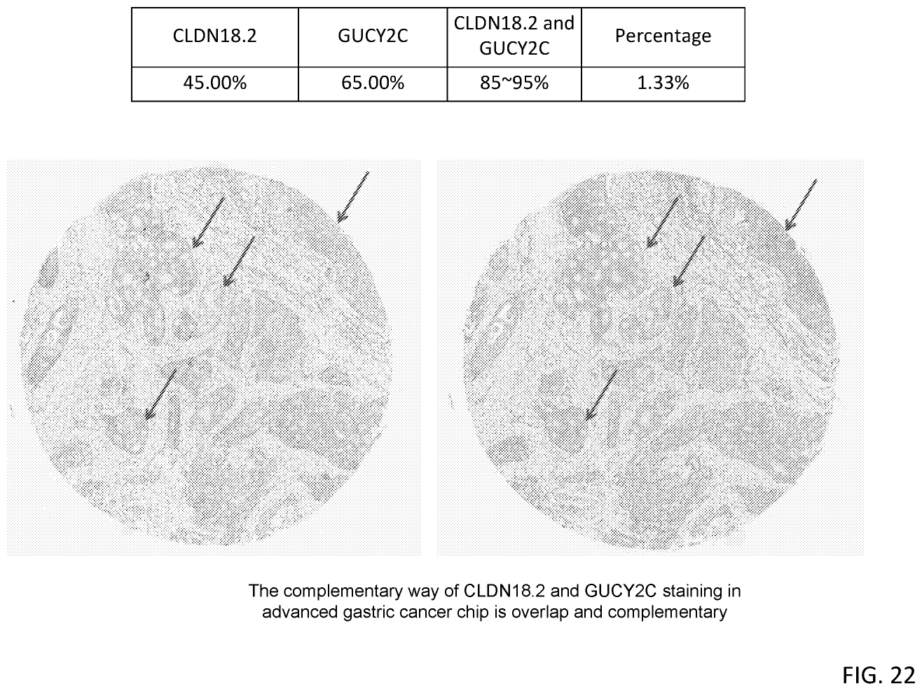

FIG. 23 shows a schematic diagram of an exemplary portion of a cell membrane comprising a bispecific (Tan (tandem)) CAR molecule.

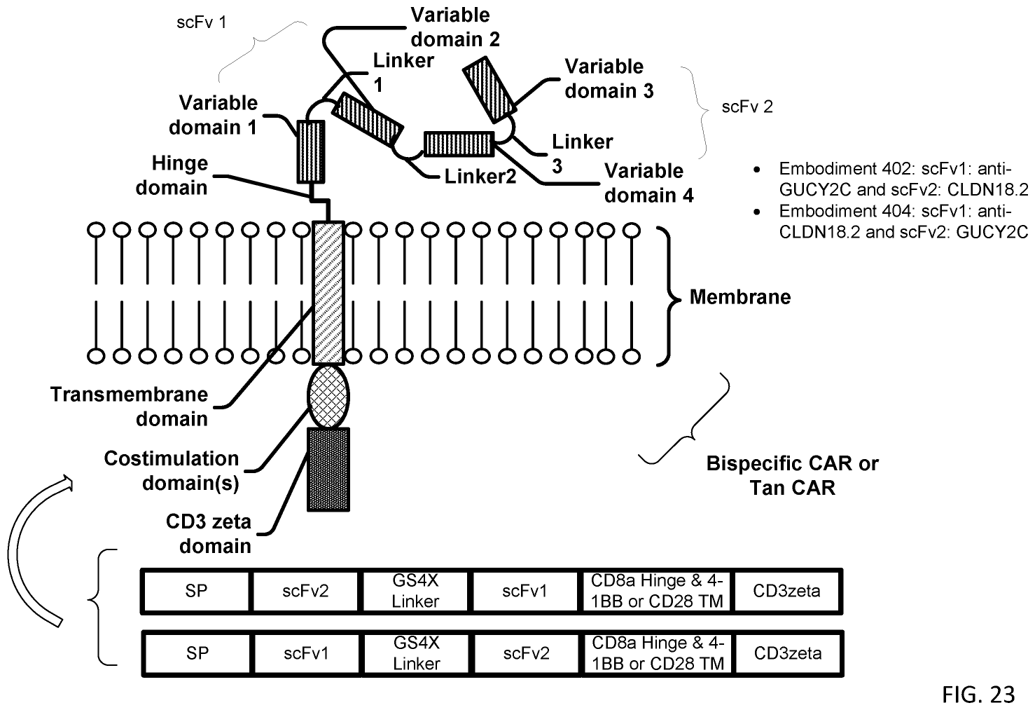

FIG. 24 shows an embodiment of mixed T cells for treating cancer.

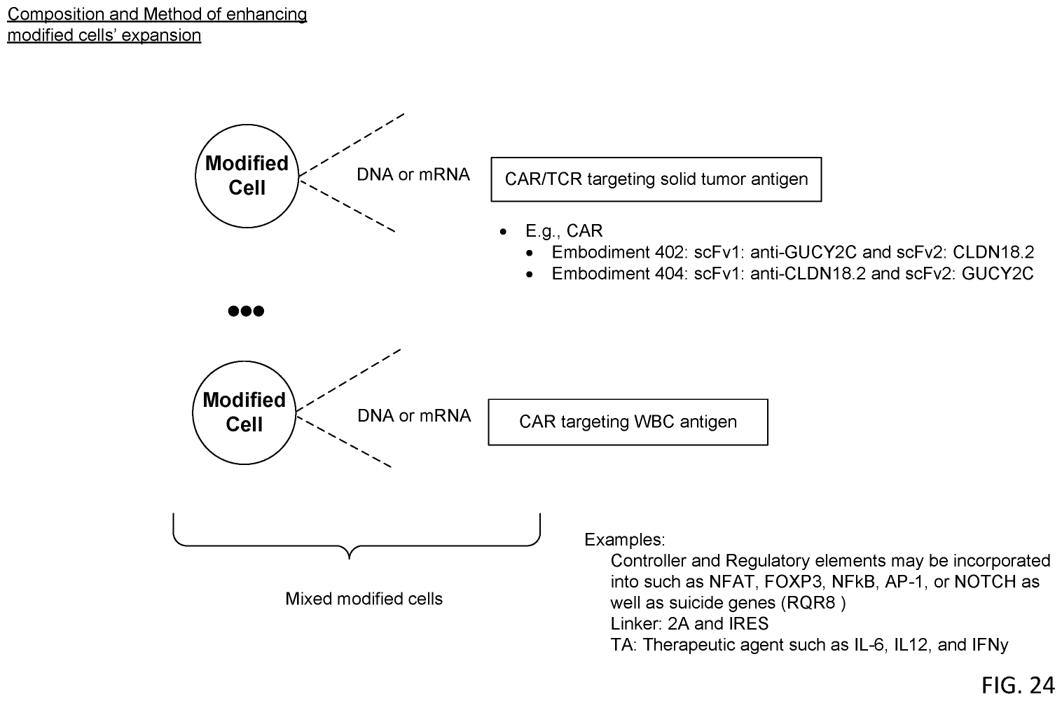

FIG. 25 shows an embodiment of mixed T cells for treating cancer.

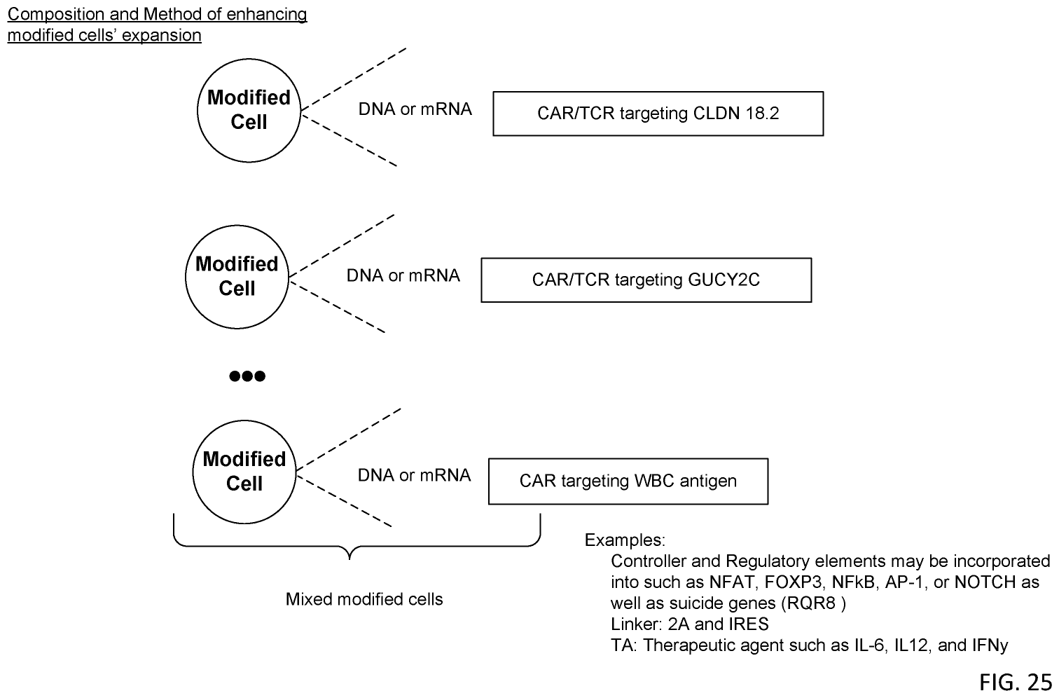

FIG. 26 shows an embodiment of polyspecific binding molecules (PBMs) for treating cancer.

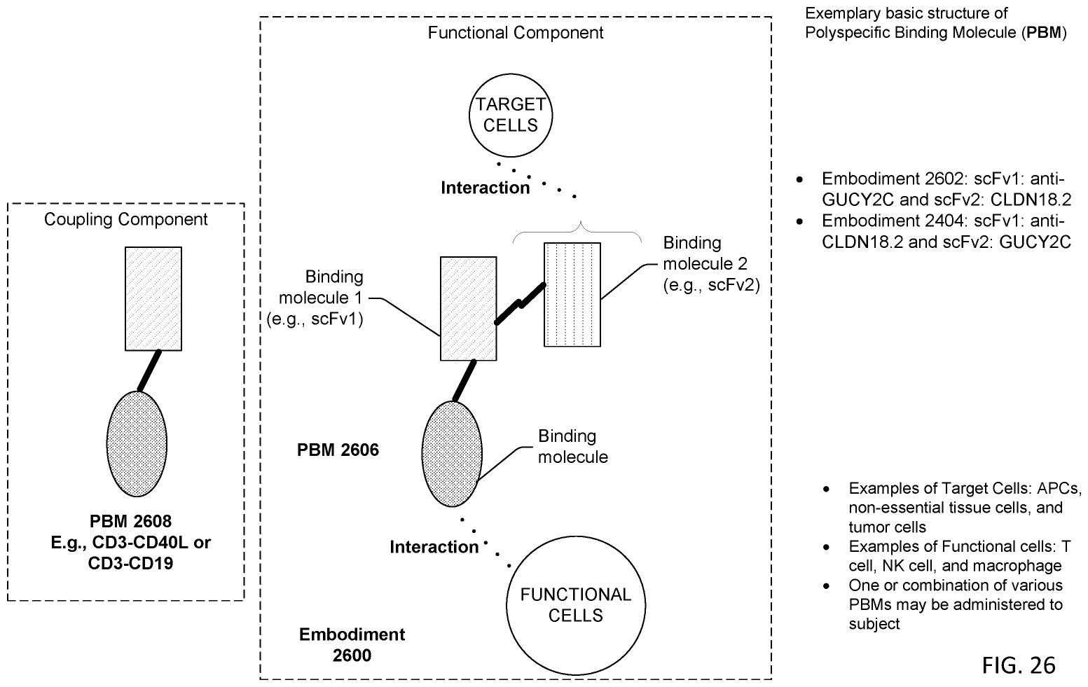

FIG. 27 shows an embodiment of polyspecific binding molecules (PBMs) for treating cancer.

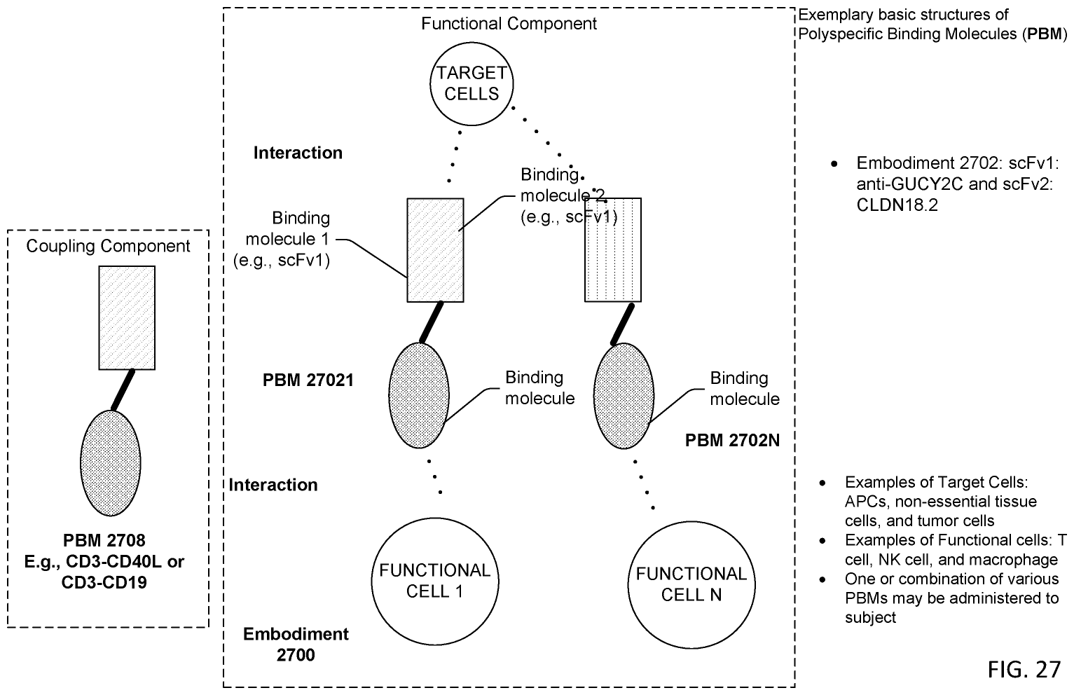

FIG. 28 shows an schematic diagram of an exemplary portion of a cell membrane comprising a bispecific CAR molecule.

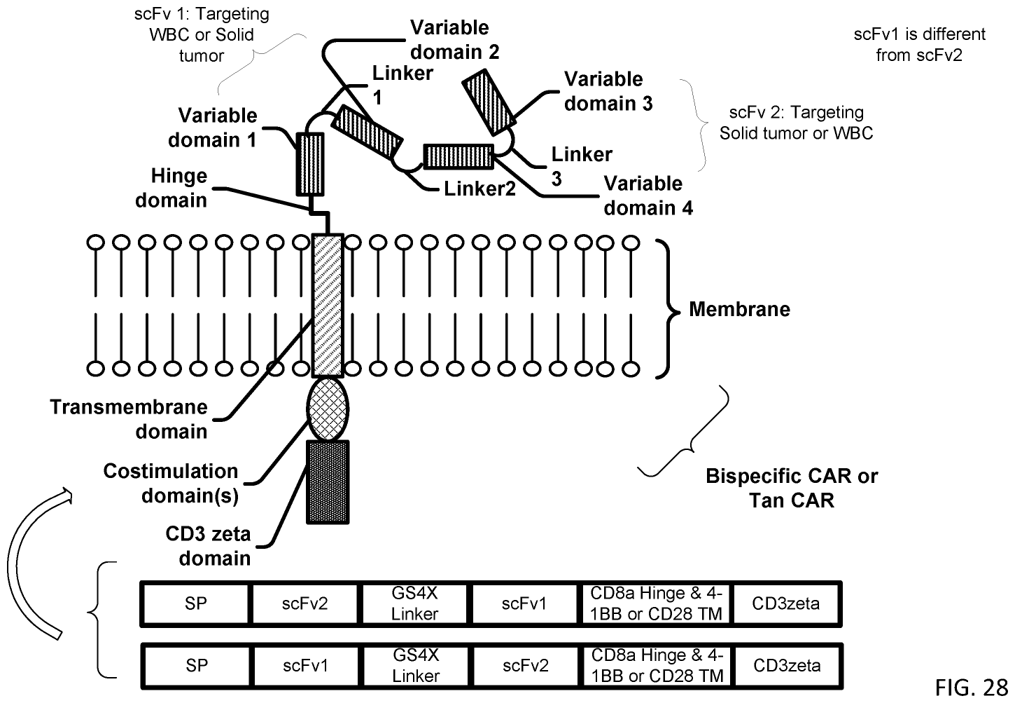

FIG. 29 shows the construct of a bispecific CAR and the results of expression assays.

FIG. 30 shows IFNγ (IFNγ) release by T cells expressing a bispecific CAR.

FIG. 31 shows results of co-culturing assay of T cells expressing a bispecific CAR and corresponding target cells.

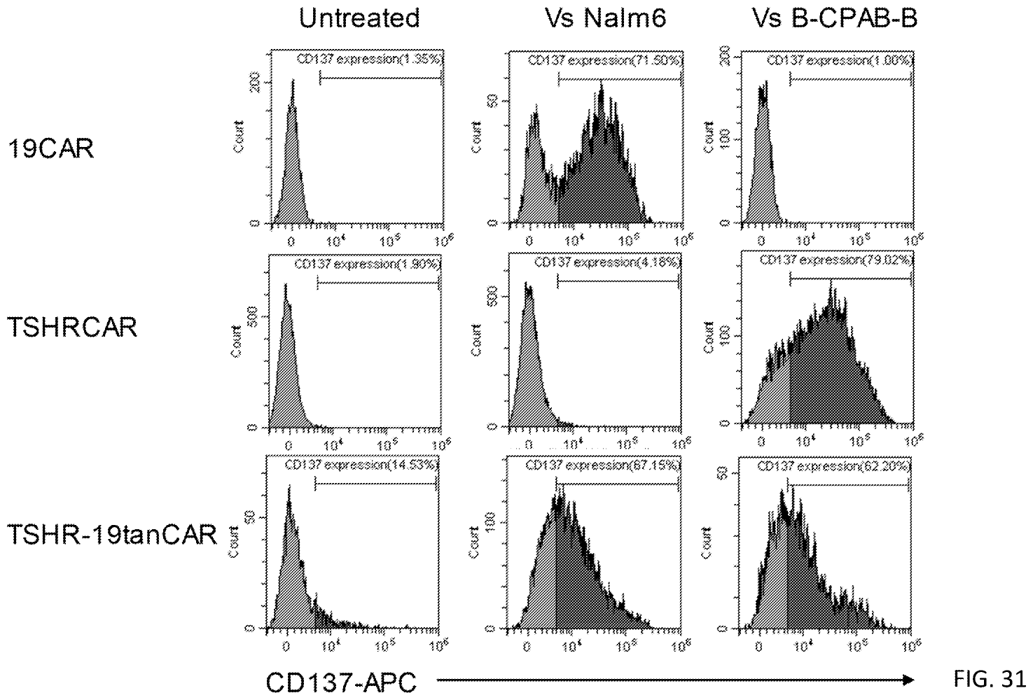

FIG. 32 shows the constructs of bispecific CARs and the results of expression assays.

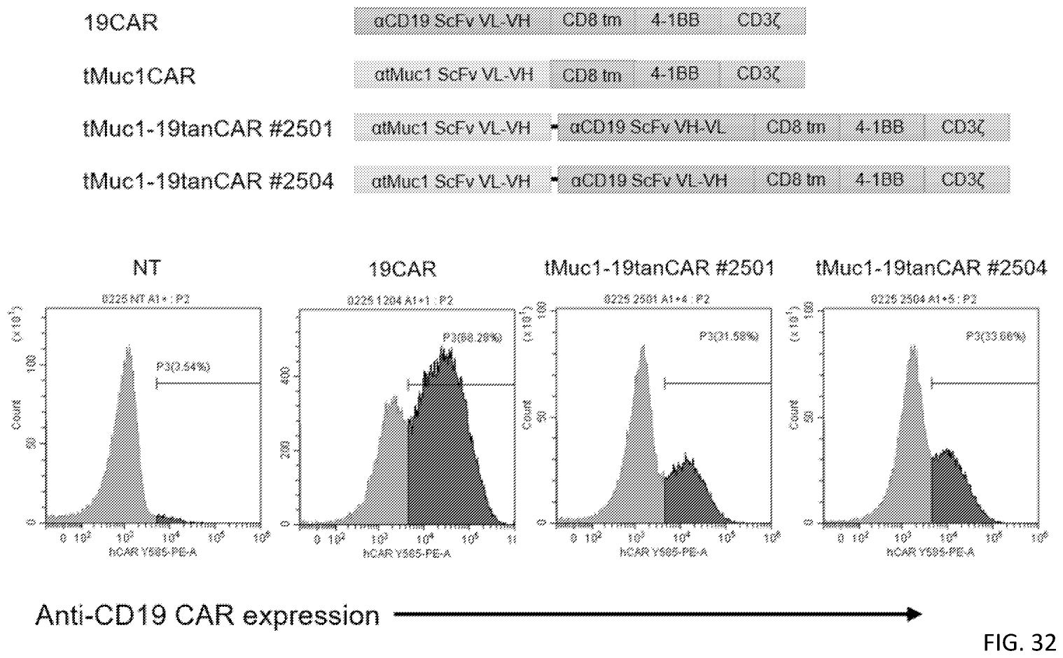

FIG. 33 shows results of co-culturing assay of T cells expressing a bispecific CAR and corresponding target cells (tumor cells).

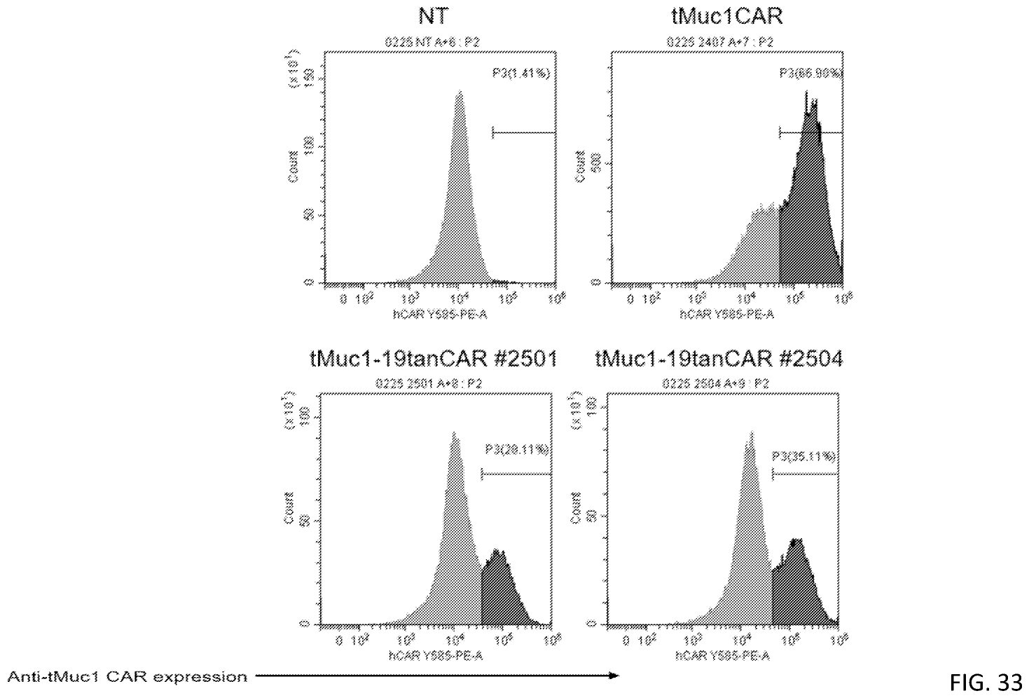

FIG. 34 shows of the construct of a bispecific CAR and results of expression assays.

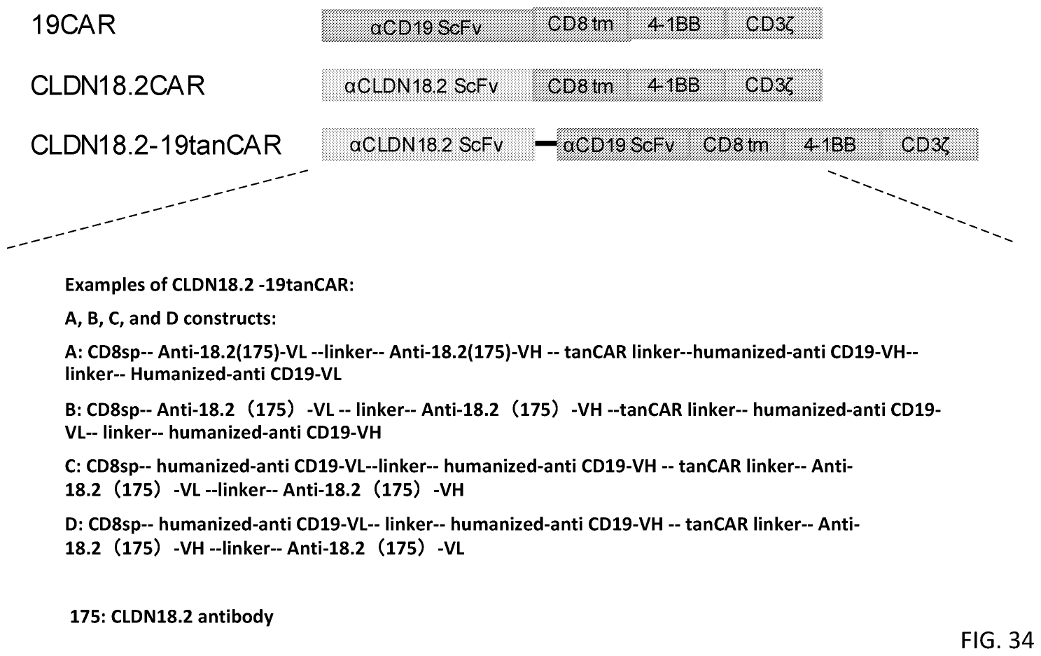

FIG. 35 shows results of an expression assay of the bispecific CAR used in the assay of FIG. 34.

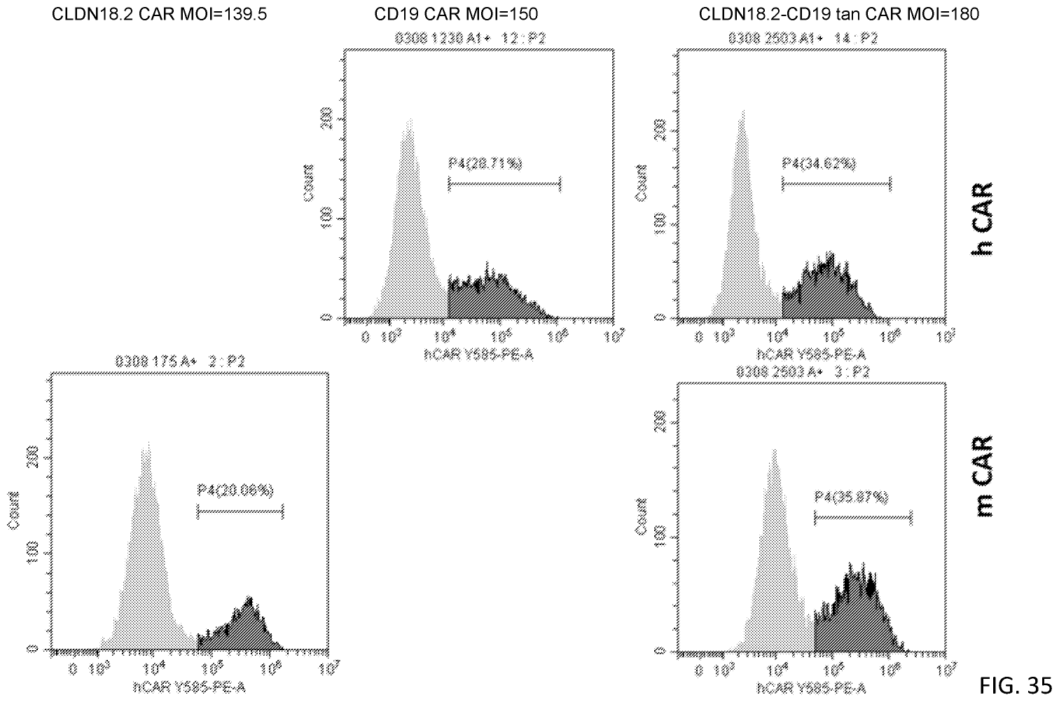

FIG. 36 shows results of IFNγ (IFNγ) release of co-culturing CAR T cells with tumor cells.

FIG. 37 shows flow cytometry results depicting CD137 expression for co-culturing of CAR T cells and tumor cells.

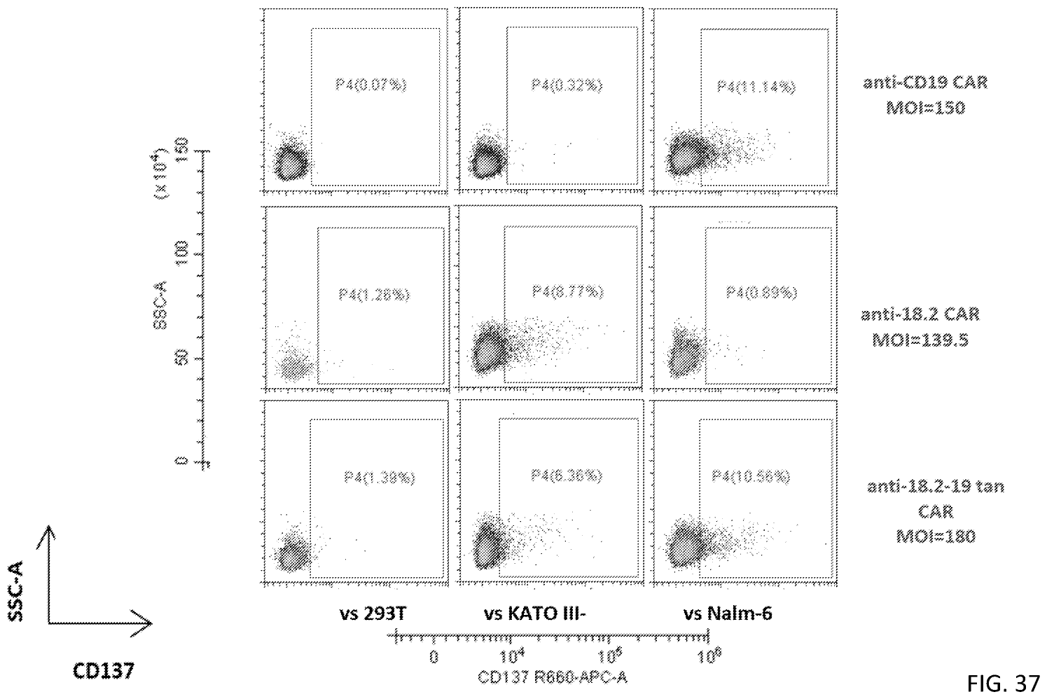

FIG. 38 shows expression of several markers (i.e., CD137, CD25, and CD40L) on CAR T cells and TanCAR T (bispecific CAR T) cells using flow cytometry analysis. Table 6 provides information regarding the cells.

FIG. 39 shows cytokine release by CAR T cells and TanCAR T cells.

FIG. 40 shows cytokine release by CAR T cells and TanCAR T cells.

FIG. 41 shows cytokine release by CAR T cells and TanCAR T cells.

FIG. 42 shows the expansion of cells in each group after 5 days of stimulation with the corresponding substrate cells.

FIG. 43 shows the results of killing assays. 6917 inhibited MCF-7 and 6921 inhibited PC3-ACPP.

FIG. 44 shows expression of several markers (i.e., CD137 and CD40L) on CART cells and TanCAR T cells. Table 6 provides information regarding the cells.

FIG. 45 shows cytokine release by CAR T cells and TanCAR T cells measured using flow cytometry analysis.

FIG. 46 shows cytokine release of various CAR T cells and TanCAR T cells in response to substrate cells.

FIG. 47 shows cytokine release of various CAR T cells and TanCAR T cells in response to substrate cells.

### DETAILED DESCRIPTION

Unless defined otherwise, all technical and scientific terms used herein have the same meaning as commonly understood by those of ordinary skill in the art to which the disclosure belongs. Although any method and material similar or equivalent to those described herein can be used in the practice or testing of the present disclosure, preferred methods and materials are described. For the purposes of the present disclosure, the following terms are defined below.

The articles “a” and “an” are used herein to refer to one or to more than one (i.e., to at least one) of the grammatical object of the article. By way of example, “an element” means one element or more than one element.

By “about” is meant a quantity, level, value, number, frequency, percentage, dimension, size, amount, weight, or length that varies by as much as 20, 15, 10, 9, 8, 7, 6, 5, 4, 3, 2 or 1% to a reference quantity, level, value, number, frequency, percentage, dimension, size, amount, weight or length.

The term “activation,” as used herein, refers to the state of a cell that has been sufficiently stimulated to induce detectable cellular proliferation. Activation can also be associated with induced cytokine production and detectable effector functions. The term “activated T cells” refers to, among other things, T cells that are undergoing cell division.

The term “antibody” is used in the broadest sense and refers to monoclonal antibodies (including full length monoclonal antibodies), polyclonal antibodies, multispecific antibodies (e.g., bispecific antibodies), and antibody fragments so long as they exhibit the desired biological activity or function. The antibodies in the present disclosure may exist in a variety of forms including, for example, polyclonal antibodies; monoclonal antibodies; Fv, Fab, Fab′, and F(ab′)2 fragments; as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragments” refers to a portion of a full-length antibody, for example, the antigen binding or variable region of the antibody. Other examples of antibody fragments include Fab, Fab′, F(ab′)2, and Fv fragments; diabodies; linear antibodies; single-chain antibody molecules; and multispecific antibodies formed from antibody fragments.

The term “Fv” refers to the minimum antibody fragment, which contains a complete antigen-recognition and -binding site. This fragment consists of a dimer of one heavy- and one light-chain variable region domain in tight, non-covalent association. From the folding of these two domains emanates six hypervariable loops (3 loops each from the H and L chain) that contribute amino acid residues for antigen binding and confer antigen binding specificity to the antibody. However, even a single variable domain (or half of an Fv including only three complementarity determining regions (CDRs) specific for an antigen) has the ability to recognize and bind antigen, although at a lower affinity than the entire binding site (the dimer).

An “antibody heavy chain,” as used herein, refers to the larger of the two types of polypeptide chains present in all antibody molecules in their naturally occurring conformations. An “antibody light chain,” as used herein, refers to the smaller of the two types of polypeptide chains present in all antibody molecules in their naturally occurring conformations. K and A light chains refer to the two major antibody light chain isotypes.

The term “synthetic antibody” refers to an antibody that is generated using recombinant DNA technology, such as, for example, an antibody expressed by a bacteriophage. The term also includes an antibody that has been generated by the synthesis of a DNA molecule encoding the antibody and the expression of the DNA molecule to obtain the antibody or to obtain an amino acid encoding the antibody. Synthetic DNA is obtained using technology that is available and well known in the art.

The term “antigen” refers to a molecule that provokes an immune response, which may involve either antibody production, or the activation of specific immunologically competent cells, or both. Antigens include any macromolecule, including all proteins or peptides or molecules derived from recombinant or genomic DNA. For example, DNA including a nucleotide sequence or a partial nucleotide sequence encoding a protein or peptide that elicits an immune response, and therefore, encodes an “antigen” as the term is used herein. An antigen need not be encoded solely by a full-length nucleotide sequence of a gene. An antigen can be generated, synthesized, or derived from a biological sample, including a tissue sample, a tumor sample, a cell, or a biological fluid.

The term “anti-tumor effect,” as used herein, refers to a biological effect associated with a decrease in tumor volume, a decrease in the number of tumor cells, a decrease in the number of metastases, decrease in tumor cell proliferation, decrease in tumor cell survival, an increase in life expectancy of a subject having tumor cells, or amelioration of various physiological symptoms associated with the cancerous condition. An “anti-tumor effect” can also be manifested by the ability of the peptides, polynucleotides, cells, and antibodies in the prevention of the occurrence of tumors in the first place.

The term “auto-antigen” refers to an endogenous antigen mistakenly recognized by the immune system as being foreign. Auto-antigens include cellular proteins, phosphoproteins, cellular surface proteins, cellular lipids, nucleic acids, glycoproteins, including cell surface receptors.

The term “autologous” is used to describe a material derived from a subject that is subsequently re-introduced into the same subject.

The term “allogeneic” is used to describe a graft derived from a different subject of the same species. As an example, a donor subject may be related or unrelated to the recipient subject, but the donor subject has immune system markers which are similar to the recipient subject.

The term “xenogeneic” is used to describe a graft derived from a subject of a different species. As an example, the donor subject is from a different species than a recipient subject, and the donor subject and the recipient subject can be genetically and immunologically incompatible.

The term “cancer” is used to refer to a disease characterized by the rapid and uncontrolled growth of aberrant cells. Cancer cells can spread locally or through the bloodstream and lymphatic system to other parts of the body. Examples of various cancers include breast cancer, prostate cancer, ovarian cancer, cervical cancer, skin cancer, pancreatic cancer, colorectal cancer, renal cancer, liver cancer, brain cancer, lymphoma, leukemia, lung cancer, and the like.

Throughout this specification, unless the context requires otherwise, the words “comprise,” “includes,” and “including” will be understood to imply the inclusion of a stated step or element or group of steps or elements but not the exclusion of any other step or element or group of steps or elements.

The phrase “consisting of” is meant to include, and is limited to, whatever follows the phrase “consisting of.” Thus, the phrase “consisting of” indicates that the listed elements are required or mandatory and that no other elements may be present.

The phrase “consisting essentially of” is meant to include any element listed after the phrase and can include other elements that do not interfere with or contribute to the activity or action specified in the disclosure for the listed elements. Thus, the phrase “consisting essentially of” indicates that the listed elements are required or mandatory, but those other elements are optional and may or may not be present depending upon whether or not they affect the activity or action of the listed elements.

The terms “complementary” and “complementarity” refer to polynucleotides (i.e., a sequence of nucleotides) related by the base-pairing rules. For example, the sequence “A-G-T” is complementary to the sequence “T-C-A.” Complementarity may be “partial,” in which only some of the nucleic acids' bases are matched according to the base-pairing rules, or there may be “complete” or “total” complementarity between the nucleic acids. The degree of complementarity between nucleic acid strands has significant effects on the efficiency and strength of hybridization between nucleic acid strands.

The term “corresponds to” or “corresponding to” refers to (a) a polynucleotide having a nucleotide sequence that is substantially identical or complementary to all or a portion of a reference polynucleotide sequence or encoding an amino acid sequence identical to an amino acid sequence in a peptide or protein, or (b) a peptide or polypeptide having an amino acid sequence that is substantially identical to a sequence of amino acids in a reference peptide or protein.

The term “co-stimulatory ligand” refers to a molecule on an antigen-presenting cell (e.g., an APC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including at least one of proliferation, activation, differentiation, and other cellular responses. A co-stimulatory ligand can include B7-1 (CD80), B7-2 (CD86), PD-L1, PD-L2, 4-1BBL, OX40L, inducible co-stimulatory ligand (ICOS-L), intercellular adhesion molecule (ICAM), CD300L, CD40, CD70, CD83, HLA-G, MICA, MICB, HVEM, lymphotoxin beta receptor, 3/TR6, ILT3, ILT4, HVEM, a ligand for CD7, an agonist or antibody that binds the Toll ligand receptor, and a ligand that specifically binds with B7-H3. A co-stimulatory ligand also includes, inter alia, an agonist or an antibody that specifically binds with a co-stimulatory molecule present on a T cell, such as CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds CD83.

The term “co-stimulatory molecule” refers to the cognate binding partner on a T cell that specifically binds with a co-stimulatory ligand, thereby mediating a co-stimulatory response by the T cell, such as proliferation. Co-stimulatory molecules include an MHC class I molecule, BTLA, and a Toll-like receptor.

The term “co-stimulatory signal” refers to a signal, which in combination with a primary signal, such as TCR/CD3 ligation, leads to T cell proliferation and/or upregulation or downregulation of key molecules.

The terms “disease” and “condition” may be used interchangeably or may be different in that the particular malady or condition may not have a known causative agent (so that etiology has not yet been worked out), and it is therefore not yet recognized as a disease but only as an undesirable condition or syndrome, wherein a more or less specific set of symptoms have been identified by clinicians. The term “disease” is a state of health of a subject wherein the subject cannot maintain homeostasis and wherein if the disease is not ameliorated, then the subject's health continues to deteriorate. In contrast, a “disorder” in a subject is a state of health in which the animal is able to maintain homeostasis, but in which the animal's state of health is less favorable than it would be in the absence of the disorder. Left untreated, a disorder does not necessarily cause a further decrease in the animal's state of health.

The term “effective” refers to adequate to accomplish a desired, expected, or intended result. For example, an “effective amount” in the context of treatment may be an amount of a compound sufficient to produce a therapeutic or prophylactic benefit.

The term “encoding” refers to the inherent property of specific sequences of nucleotides in a polynucleotide, such as a gene, a cDNA, or an mRNA, to serve as a template for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence (except that a “T” is replaced by a “U”) and is usually provided in sequence listings, and the non-coding strand, used as the template for transcription of a gene or cDNA, can be referred to as encoding the protein or other product of that gene or cDNA.

The term “exogenous” refers to a molecule that does not naturally occur in a wild-type cell or organism but is typically introduced into the cell by molecular biological techniques. Examples of exogenous polynucleotides include vectors, plasmids, and/or man-made nucleic acid constructs encoding the desired protein. With regard to polynucleotides and proteins, the term “endogenous” or “native” refers to naturally occurring polynucleotide or amino acid sequences that may be found in a given wild-type cell or organism. Also, a particular polynucleotide sequence that is isolated from a first organism and transferred to a second organism by molecular biological techniques is typically considered an “exogenous” polynucleotide or amino acid sequence with respect to the second organism. In specific embodiments, polynucleotide sequences can be “introduced” by molecular biological techniques into a microorganism that already contains such a polynucleotide sequence, for instance, to create one or more additional copies of an otherwise naturally-occurring polynucleotide sequence, and thereby facilitate overexpression of the encoded polypeptide.

The term “expression” refers to the transcription and/or translation of a particular nucleotide sequence driven by its promoter.

The term “expression vector” refers to a vector including a recombinant polynucleotide including expression control (regulatory) sequences operably linked to a nucleotide sequence to be expressed. An expression vector includes sufficient cis-acting elements for expression; other elements for expression can be supplied by the host cell or in an in vitro expression system. Expression vectors include all those known in the art, such as cosmids, plasmids (e.g., naked or contained in liposomes), and viruses (e.g., lentiviruses, retroviruses, adenoviruses, and adeno-associated viruses) that incorporate the recombinant polynucleotide.

The term “homologous” refers to sequence similarity or sequence identity between two polypeptides or between two polynucleotides when a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared to 100. For example, if 6 of 10 of the positions in two sequences are matched or homologous, then the two sequences are 60% homologous. By way of example, the DNA sequences ATTGCC and TATGGC share 50% homology. A comparison is made when two sequences are aligned to give maximum homology.

The term “immunoglobulin” or “Ig” refers to a class of proteins, which function as antibodies. The five members included in this class of proteins are IgA, IgG, IgM, IgD, and IgE. IgA is the primary antibody that is present in body secretions, such as saliva, tears, breast milk, gastrointestinal secretions, and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses and is important in defense against bacteria and viruses. IgD is the immunoglobulin that has no known antibody function but may serve as an antigen receptor. IgE is the immunoglobulin that mediates immediate hypersensitivity by causing the release of mediators from mast cells and basophils upon exposure to the allergen.

The term “isolated” refers to a material that is substantially or essentially free from components that normally accompany it in its native state. The material can be a cell or a macromolecule, such as a protein or nucleic acid. For example, an “isolated polynucleotide,” as used herein, refers to a polynucleotide, which has been purified from the sequences which flank it in a naturally occurring state, e.g., a DNA fragment that has been removed from the sequences that are normally adjacent to the fragment. Alternatively, an “isolated peptide” or an “isolated polypeptide” and the like, as used herein, refer to in vitro isolation and/or purification of a peptide or polypeptide molecule from its natural cellular environment and from association with other components of the cell.

The term “substantially purified” refers to a material that is substantially free from components that are normally associated with it in its native state. For example, a substantially purified cell refers to a cell that has been separated from other cell types with which it is normally associated in its naturally occurring or native state. In some instances, a population of substantially purified cells refers to a homogenous population of cells. In other instances, this term refers simply to a cell that has been separated from the cells with which they are naturally associated in their natural state. In embodiments, the cells are cultured in vitro. In embodiments, the cells are not cultured in vitro.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some versions contain an intron(s).

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. Moreover, the use of lentiviruses enables the integration of genetic information into the host chromosome, resulting in stably transduced genetic information. HIV, SIV, and FIV are all examples of lentiviruses. Vectors derived from lentiviruses offer the means to achieve significant levels of gene transfer in vivo.

The term “modulating” refers to mediating a detectable increase or decrease in the level of a response in a subject compared with the level of a response in the subject in the absence of a treatment or compound and/or compared with the level of a response in an otherwise identical but untreated subject. The term encompasses perturbing and/or affecting a native signal or response, thereby mediating a beneficial therapeutic response in a subject, preferably a human.

Nucleic acid is “operably linked” when it is placed into a functional relationship with another nucleic acid. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence, or a ribosome binding site is operably linked to a coding sequence if it is positioned so as to facilitate translation.

The term “under transcriptional control” refers to a promoter being operably linked to and in the correct location and orientation in relation to a polynucleotide to control (regulate) the initiation of transcription by RNA polymerase and expression of the polynucleotide.

The term “overexpressed” tumor antigen or “overexpression” of the tumor antigen is intended to indicate an abnormal level of expression of the tumor antigen in a cell from a disease area such as a solid tumor within a specific tissue or organ of the patient relative to the level of expression in a normal cell from that tissue or organ. Patients having a solid tumor or a hematological malignancy characterized by overexpression of the tumor antigen can be determined by standard assays known in the art.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme), astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma, and brain metastases).

A solid tumor antigen is an antigen expressed on a solid tumor. In embodiments, solid tumor antigens are also expressed at low levels on healthy tissue. Examples of solid tumor antigens and their related disease tumors are provided in Table 1.

TABLE 1

| Solid Tumor | |
|---|---|
| antigen | Disease tumor |
| PRLR | Breast Cancer |
| CLCA1 | colorectal Cancer |
| MUC12 | colorectal Cancer |
| GUCY2C | colorectal Cancer |
| GPR35 | colorectal Cancer |
| CR1L | Gastric Cancer |
| MUC 17 | Gastric Cancer |
| TMPRSS11B | esophageal Cancer |
| MUC21 | esophageal Cancer |
| TMPRSS11E | esophageal Cancer |
| CD207 | bladder Cancer |
| SLC30A8 | pancreatic Cancer |
| CFC1 | pancreatic Cancer |
| SLC12A3 | Cervical Cancer |
| SSTR1 | Cervical tumor |
| GPR27 | Ovary tumor |
| FZD10 | Ovary tumor |
| TSHR | Thyroid Tumor |
| SIGLEC15 | Urothelial cancer |
| SLC6A3 | Renal cancer |
| KISS1R | Renal cancer |
| QRFPR | Renal cancer: |
| GPR119 | Pancreatic cancer |
| CLDN6 | Endometrial cancer/Urothelial cancer |
| UPK2 | Urothelial cancer (including bladder cancer) |
| ADAM12 | Breast cancer, pancreatic cancer and the like |
| SLC45A3 | Prostate cancer |
| ACPP | Prostate cancer |
| MUC21 | Esophageal cancer |
| MUC16 | Ovarian cancer |
| MS4A12 | Colorectal cancer |
| ALPP | Endometrial cancer |
| CEA | Colorectal carcinoma |
| EphA2 | Glioma |
| FAP | Mesotelioma |
| GPC3 | Lung squamous cell carcinoma |
| IL-13Rα2 | Glioma |
| Mesothelin | Metastatic cancer |
| PSMA | Prostate cancer |
| ROR1 | Breast lung carcinoma |
| VEGFR-II | Metastatic cancer |
| GD2 | Neuroblastoma |
| FR-α | Ovarian carcinoma |
| ErbB2 | Carcinomasb |
| EpCAM | Carcinomasa |
| EGFRvIII | Glioma-Glioblastoma |
| EGFR | Glioma-NSCL cancer |
| tMUC 1 | Cholangiocarcinoma, Pancreatic cancer, Breast Cancer |
| PSCA | pancreas, stomach, or prostate cancer |

The term “parenteral administration” of the composition includes, e.g., subcutaneous (s.c.), intravenous (i.v.), intramuscular (i.m.), intrasternal injection, or infusion techniques.

The terms “patient,” “subject,” and “individual,” and the like are used interchangeably herein and refer to any human or animal, amenable to the methods described herein. In embodiments, the patient, subject, or individual is a human or animal. In embodiments, the term “subject” is intended to include living organisms in which an immune response can be elicited (e.g., mammals). Examples of subjects include humans, and animals, such as dogs, cats, mice, rats, and transgenic species thereof.

A subject in need of treatment or in need thereof includes a subject having a disease, condition, or disorder that needs to be treated. A subject in need thereof also includes a subject that needs treatment for the prevention of a disease, condition, or disorder.

The term “polynucleotide” or “nucleic acid” refers to mRNA, RNA, cRNA, rRNA, cDNA, or DNA. The term typically refers to a polymeric form of nucleotides of at least 10 bases in length, either ribonucleotides or deoxynucleotides, or a modified form of either type of nucleotide. The term includes all forms of nucleic acids, including single and double-stranded forms of nucleic acids.

The terms “polynucleotide variant” and “variant,” and the like refer to polynucleotides displaying substantial sequence identity with a reference polynucleotide sequence or polynucleotides that hybridize with a reference sequence under stringent conditions that are defined hereinafter. These terms also encompass polynucleotides that are distinguished from a reference polynucleotide by the addition, deletion, or substitution of at least one nucleotide. Accordingly, the terms “polynucleotide variant” and “variant” include polynucleotides in which one or more nucleotides have been added or deleted, or replaced with different nucleotides. In this regard, it is well understood in the art that certain alterations inclusive of mutations, additions, deletions, and substitutions can be made to a reference polynucleotide whereby the altered polynucleotide retains the biological function or activity of the reference polynucleotide or has increased activity in relation to the reference polynucleotide (i.e., optimized). Polynucleotide variants include, for example, polynucleotides having at least 50% (and at least 51% to at least 99% and all integer percentages in between, e.g., 90%, 95%, or 98%) sequence identity with a reference polynucleotide sequence described herein. The terms “polynucleotide variant” and “variant” also include naturally-occurring allelic variants and orthologs.

The terms “polypeptide,” “polypeptide fragment,” “peptide,” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally occurring amino acid polymers. In embodiments, polypeptides may include enzymatic polypeptides, or “enzymes,” which typically catalyze (i.e., increase the rate of) various chemical reactions.

The term “polypeptide variant” refers to polypeptides that are distinguished from a reference polypeptide sequence by the addition, deletion, or substitution of at least one amino acid residue. In embodiments, a polypeptide variant is distinguished from a reference polypeptide by one or more substitutions, which may be conservative or non-conservative. In embodiments, the polypeptide variant comprises conservative substitutions, and, in this regard, it is well understood in the art that some amino acids may be changed to others with broadly similar properties without changing the nature of the activity of the polypeptide. Polypeptide variants also encompass polypeptides in which one or more amino acids have been added or deleted, or replaced with different amino acid residues.

The term “promoter” refers to a DNA sequence recognized by the synthetic machinery of the cell or introduced synthetic machinery required to initiate the specific transcription of a polynucleotide sequence. The term “expression control (regulatory) sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

The term “bind,” “binds,” or “interacts with” refers to a molecule recognizing and adhering to a second molecule in a sample or organism but does not substantially recognize or adhere to other structurally unrelated molecules in the sample. The term “specifically binds,” as used herein with respect to an antibody, refers to an antibody that recognizes a specific antigen but does not substantially recognize or bind other molecules in a sample. For example, an antibody that specifically binds an antigen from one species may also bind that antigen from one or more species. But, such cross-species reactivity does not itself alter the classification of an antibody as specific. In another example, an antibody that specifically binds an antigen may also bind different allelic forms of the antigen. However, such cross reactivity does not itself alter the classification of an antibody as specific. In some instances, the terms “specific binding” or “specifically binding” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds a specific protein structure rather than to any protein. If an antibody is specific for epitope “A,” the presence of a molecule containing epitope A (or free, unlabeled A), in a reaction containing labeled “A” and the antibody, will reduce the amount of labeled A bound to the antibody.

By “statistically significant,” it is meant that the result was unlikely to have occurred by chance. Statistical significance can be determined by any method known in art. Commonly used measures of significance include the p-value, which is the frequency or probability with which the observed event would occur if the null hypothesis were true. If the obtained p-value is smaller than the significance level, then the null hypothesis is rejected. In simple cases, the significance level is defined at a p-value of 0.05 or less. A “decreased” or “reduced” or “lesser” amount is typically a “statistically significant” or a physiologically significant amount and may include a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein.

The term “stimulation” refers to a primary response induced by binding of a stimulatory molecule (e.g., a TCR/CD3 complex) with its cognate ligand, thereby mediating a signal transduction event, such as signal transduction via the TCR/CD3 complex. Stimulation can mediate altered expression of certain molecules, such as downregulation of TGF-β and/or reorganization of cytoskeletal structures.

The term “stimulatory molecule” refers to a molecule on a T cell that specifically binds a cognate stimulatory ligand present on an antigen-presenting cell. For example, a functional signaling domain derived from a stimulatory molecule is the zeta chain associated with the T cell receptor complex. The stimulatory molecule includes a domain responsible for signal transduction.

The term “stimulatory ligand” refers to a ligand that when present on an antigen-presenting cell (e.g., an APC, a dendritic cell, a B-cell, and the like.) can specifically bind with a cognate binding partner (referred to herein as a “stimulatory molecule”) on a cell, for example, a T cell, thereby mediating a primary response by the T cell, including activation, initiation of an immune response, proliferation, and similar processes. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “therapeutic” refers to treatment and/or prophylaxis. A therapeutic effect is obtained by suppression, remission, or eradication of a disease state or alleviating the symptoms of a disease state.

The term “therapeutically effective amount” refers to the amount of the subject compound that will elicit the biological or medical response of a tissue, system, or subject that is being sought by the researcher, veterinarian, medical doctor, or another clinician. The term “therapeutically effective amount” includes that amount of a compound that, when administered, is sufficient to prevent the development of, or alleviate to some extent, one or more of the signs or symptoms of the disorder or disease being treated. The therapeutically effective amount will vary depending on the compound, the disease, and its severity, and the age, weight, etc., of the subject to be treated.

The term “treat a disease” refers to the reduction of the frequency or severity of at least one sign or symptom of a disease or disorder experienced by a subject.

The term “transfected” or “transformed” or “transduced” refers to a process by which an exogenous nucleic acid is transferred or introduced into the host cell. A “transfected” or “transformed” or “transduced” cell is one that has been transfected, transformed, or transduced with exogenous nucleic acid. The cell includes the primary subject cell and its progeny.

The term “vector” refers to a polynucleotide that comprises an isolated nucleic acid and which can be used to deliver the isolated nucleic acid to the interior of a cell. Numerous vectors are known in the art, including linear polynucleotides, polynucleotides associated with ionic or amphiphilic compounds, plasmids, and viruses. Thus, the term “vector” includes an autonomously replicating plasmid or a virus. The term also includes non-plasmid and non-viral compounds which facilitate the transfer of nucleic acid into cells, such as, for example, polylysine compounds, liposomes, and the like. Examples of viral vectors include adenoviral vectors, adeno-associated virus vectors, retroviral vectors, and others. For example, lentiviruses are complex retroviruses, which, in addition to the common retroviral genes gag, pol, and env, contain other genes with regulatory or structural functions. Lentiviral vectors are well known in art. Some examples of lentivirus include the Human Immunodeficiency Viruses: HIV-1, HIV-2, and the Simian Immunodeficiency Virus: SIV. Lentiviral vectors have been generated by multiply attenuating the HIV virulence genes, for example, the genes env, vif, vpr, vpu, and nef are deleted, making the vector biologically safe.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, and 6. This applies regardless of the breadth of the range.

A “chimeric antigen receptor” (CAR) molecule is a recombinant polypeptide including at least an extracellular domain, a transmembrane domain, and a cytoplasmic domain or intracellular domain. In embodiments, the domains of the CAR are on the same polypeptide chain, for example, a chimeric fusion protein. In embodiments, the domains are on different polypeptide chains, for example, the domains are not contiguous.

The extracellular domain of a CAR molecule includes an antigen binding domain. The antigen binding domain is for expanding and/or maintaining the modified cells, such as a CAR T cell, or for killing a tumor cell, such as a solid tumor. In embodiments, the antigen binding domain for expanding and/or maintaining modified cells binds an antigen, for example, a cell surface molecule or marker, on the surface of a WBC. In embodiments, the WBC is a granulocyte, monocyte, and or lymphocyte. In embodiments, the WBC is a lymphocyte, for example, a B cell. In embodiments, the WBC is a B cell. In embodiments, the cell surface molecule of a B cell includes CD19, CD22, CD20, BCMA, CDS, CD7, CD2, CD16, CD56, CD30, CD14, CD68, CD11b, CD18, CD169, CD1c, CD33, CD38, CD138, or CD13. In embodiments, the cell surface molecule of the B cell is CD19, CD20, CD22, or BCMA. In embodiments, the cell surface molecule of the B cell is CD19.

In embodiments, the antigen binding domain for killing a tumor binds an antigen on the surface of a tumor, for example, a tumor antigen or tumor marker. Tumor antigens are proteins that are produced by tumor cells that elicit an immune response, particularly T cell-mediated immune responses. Tumor antigens are well known in the art and include, for example, tumor-associated MUC1 (tMUC1), a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, surviving, telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor, CD19, and mesothelin. For example, when the tumor antigen is CD19, the CAR thereof can be referred to as CD19 CAR, which is a CAR molecule that includes an antigen binding domain that binds CD19.

In embodiments, the extracellular antigen binding domain of a CAR includes at least one scFv or at least a single domain antibody. As an example, there can be two scFvs on a CAR. The scFv includes a light chain variable (VL) region and a heavy chain variable (VH) region of a target antigen-specific monoclonal antibody joined by a flexible linker. Single chain variable region fragments can be made by linking light and/or heavy chain variable regions by using a short linking peptide (Bird et al., Science 242:423-426, 1988). An example of a linking peptide is the GS linker having the amino acid sequence (GGGGS)3 (SEQ ID NO: 131), which bridges approximately 3.5 nm between the carboxy terminus of one variable region and the amino terminus of the other variable region. Linkers of other sequences have been designed and used (Bird et al., 1988, supra). In general, linkers can be short, flexible polypeptides and preferably comprised of about 20 or fewer amino acid residues. The single-chain variants can be produced either recombinantly or synthetically. For synthetic production of scFv, an automated synthesizer can be used. For recombinant production of scFv, a suitable plasmid containing a polynucleotide that encodes the scFv can be introduced into a suitable host cell, either eukaryotic, such as yeast, plant, insect, or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

The cytoplasmic domain of the CAR molecules described herein includes one or more co-stimulatory domains and one or more signaling domains. The co-stimulatory and signaling domains function to transmit the signal and activate molecules, such as T cells, in response to antigen binding. The one or more co-stimulatory domains are derived from stimulatory molecules and/or co-stimulatory molecules, and the signaling domain is derived from a primary signaling domain, such as the CD3 zeta domain. In embodiments, the signaling domain further includes one or more functional signaling domains derived from a co-stimulatory molecule. In embodiments, the co-stimulatory molecules are cell surface molecules (other than antigens receptors or their ligands) that are required for activating a cellular response to an antigen.

In embodiments, the co-stimulatory domain includes the intracellular domain of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83, or any combination thereof. In embodiments, the signaling domain includes a CD3 zeta domain derived from a T cell receptor.

The CAR molecules described herein also include a transmembrane domain. The incorporation of a transmembrane domain in the CAR molecules stabilizes the molecule. In embodiments, the transmembrane domain of the CAR molecules is the transmembrane domain of a CD28 or 4-1BB molecule.

Between the extracellular domain and the transmembrane domain of the CAR, there may be incorporated a spacer domain. As used herein, the term “spacer domain” generally means any oligo- or polypeptide that functions to link the transmembrane domain to the extracellular domain and/or the cytoplasmic domain on the polypeptide chain. A spacer domain may include up to 300 amino acids, preferably 10 to 100 amino acids, and most preferably 25 to 50 amino acids.

An “agonist antibody,” as used herein, is an antibody that activates a biological activity of the antigen it binds. In embodiments, during agonist antibody activation, partial dissociation of antibodies allows the antigen binding fragment (Fab) arms of a single antibody to interact with more than two receptors in a dynamic fashion, resulting in the recruitment of multiple receptor monomers into a receptor oligomer where signaling activation can be triggered. In embodiments, antigen-presenting cells (APCs) acts as a scaffold to crosslink agonist antibodies bound to a receptor (e.g., co-stimulatory receptor), leading to receptor supercluster formation and increased agonist signaling. Some proteins (such as the CD28 family and the TNF receptor family) have an agonist antibody for tumor therapy. More information about agonist antibodies and co-stimulatory molecules may be found at Nature Reviews Drug Discovery volume 17, pages 509-527 (2018), which is incorporated herein for its entirety.

Embodiments relate to a fusion protein that can be activated by these agonist antibodies to perform the specific desired functions. In the overexpressed fusion protein, the extracellular domain is a protein sequence that can be bound by the agonist antibody (that is, the extracellular domain of the antibody against the antibody), and the intracellular is the intracellular domain of another transmembrane protein. When using the agonist antibody, the extracellular domains of the fusion protein are cross-linked (as the agonist antibodies usually will cross-link them), and the intracellular domains of the fusion protein form a cluster that activates the downstream pathway. In embodiments, examples of the extracellular domains comprise the extracellular domain of OX40, CD40, 4-1BB, Her2, GITR, ICOS, EGFR, CD27, CD28, CRCX4, IL-10R, IL-12R, IL-18R1, IL-23R, GP130, or IL-15Ra. For example, Plerixafor, also known as AMD3100, is a reversible antagonist of CXCR4, which can interrupt the binding between CXCR4 and SDF-1, thereby causing HSC to be released to the peripheral blood. In embodiments, the transmembrane domain may comprise the Notch domain, and the intracellular domain can comprise a transcription factor or mitochondrial protein. Once induced via agonist antibodies or Notch, these transcription factors and/or mitochondrial proteins can perform their functions.

Embodiments relate to a polynucleotide comprising a fusion protein comprising the amino acid SEQ ID NO: 37 or one of SEQ ID NO: 110-116. Embodiments relate to a modified cell comprising the polynucleotide. In embodiments, the modified cell is a lymphocyte such as T cells, NK cells, macrophages, or dendritic cells. In embodiments, the modified cell comprises a CAR or a modified TCR.

Embodiments relate to a polynucleotide encoding a fusion protein comprising at least one of amino acid SEQ ID NO: 81-100 and 102-105. Embodiments relate to a modified cell comprising the polynucleotide. In embodiments, the modified cell is a lymphocyte such as T or NK cells. In embodiments, the modified cell comprises a CAR or a modified TCR.

Embodiments relate to a polynucleotide encoding a modified protein comprising the amino acid SEQ ID NO: 76, 77, 78, or 79. Embodiments relate to a modified cell comprising the polynucleotide and/or a dominant negative form of SIGLEC-10. In embodiments, the modified cell is a lymphocyte such as T or NK cells. In embodiments, the modified cell comprises a CAR or a modified TCR. More information on dominant-negative forms of proteins can be found in PCT Publication No: WO2020086989, which is incorporated herein in its entirety.

Embodiments relate to a polynucleotide encoding a modified protein comprising the amino acid SEQ ID NO: 72 or 74. Embodiments relate to a modified cell comprising the polynucleotide and/or a modified form of SIRPα. In embodiments, the modified cell is a lymphocyte such as T or NK cells. In embodiments, the modified cell comprises a CAR or a modified TCR.

Embodiments relate to a method of enhancing T cell response, the method comprising: introducing a polynucleotide encoding a chimeric antigen receptor (CAR) and a polynucleotide encoding an extracellular domain of CD40 into T cells to obtain modified T cells; contacting the modified T cells with an antigen that the CAR binds and a CD40 activator, thereby inducing T cell response, wherein a level of T cell response is greater than a level of T cell response induced by contacting the modified T cells with the antigen but without the CD40 activator.

It has been reported that CD40 activation promotes apoptosis of tumor cells and stimulation of the immune response, and the presence if CD40 on carcinoma cell lines may indicate that it is involved in generating tumor specific T cell responses that contribute to inhibiting tumor cell growth or killing tumor cells. CD40 activation is mediated by CD40 activators, such as CD40 ligand (CD40L) and CD40 antibodies. CD40L, also known as CD154, is a natural ligand of CD40. The interaction of CD40 and CD40L is a major component of Tcell help. CD40 antibodies have been shown to mimic the signals CD40L.

Embodiments relate to a method of reducing PD-1 expressed on CART cells, the method comprising: introducing a polynucleotide encoding a CAR and a polynucleotide encoding an extracellular domain of CD40 into T cells to obtain modified T cells; contacting the modified T cells with an antigen that the CAR binds and a CD40 activator, thereby inducing PD-1 expression, wherein a level of PD-1 expression is lower than a level of PD-1 expression induced by contacting the modified T cells with the antigen but without the CD40 activator.

Embodiments relate to a method of maintaining and/or extending naive T cell status, the method comprising: introducing a polynucleotide encoding a CAR and a polynucleotide encoding an extracellular domain of CD40 into T cells to obtain modified T cells; contacting the modified T cells with an antigen that the CAR binds and a CD40 activator, thereby inducing differentiation from naive T cells to effector T cells, wherein a level of the differentiation is lower than a level of the differentiation induced by contacting the modified T cells with the antigen but without the CD40 activator.

Embodiments relate to a population of T cells comprising a polynucleotide encoding a CAR and a polynucleotide encoding an extracellular domain of CD40 into T cells

Embodiments relate to a method of enhancing T cell response, the method comprising: introducing a polynucleotide encoding a TCR and a polynucleotide encoding an extracellular domain of CD40 into T cells to obtain modified T cells; contacting the modified T cells with an antigen that the TCR binds and a CD40 activator, thereby inducing T cell response, wherein a level of T cell response is greater than a level of T cell response induced by contacting the modified T cells with the antigen but without the CD40 activator.

Embodiments relate to a method of reducing PD-1 expressed on T cells, the method comprising: introducing a polynucleotide encoding a TCR and a polynucleotide encoding an extracellular domain of CD40 into T cells to obtain modified T cells; contacting the modified T cells with an antigen that the TCR binds and a CD40 activator, thereby inducing PD-1 expression, wherein a level of PD-1 expression is lower than a level of PD-1 expression induced by contacting the modified T cells with the antigen but without the CD40 activator.

Embodiments relate to a method of maintaining and/or extending naive T cell status, the method comprising: introducing a polynucleotide encoding a TCR and a polynucleotide encoding an extracellular domain of CD40 into T cells to obtain modified T cells; contacting the modified T cells with an antigen that the TCR binds and a CD40 activator, thereby inducing differentiation from naive T cells to effector T cells, wherein a level of the differentiation is lower than a level of the differentiation induced by contacting the modified T cells with the antigen but without the CD40 activator.

Embodiments relate to a population of T cells comprising a polynucleotide encoding a TCR and a polynucleotide encoding an extracellular domain of CD40 into T cells

In embodiments, the modified cells comprise a modified T Cell Receptor (TCR). In embodiments, the TCR is derived from spontaneously occurring tumor-specific T cells in patients. In embodiments, the TCR binds a tumor antigen. In embodiments, the tumor antigen comprises CEA, gp100, MART-1, p53, MAGE-A3, or NY-ESO-1. In embodiments, the TCR comprises TCRγ and TCRδ chains, TCRα and TCRβ chains, or a combination thereof. In embodiments, the modified cells are derived from tumor infiltrating lymphocytes (TILs).

In embodiments, the CD40 activator is a CD40 ligand or a CD40 agonist. In embodiments, the polynucleotide encoding the extracellular domain of CD40 comprises SEQ ID NO: 36.

In embodiments, the T cell response comprises T cell expansion and/or T cell activation.

In embodiments, the CAR comprises an antigen binding domain, a transmembrane domain, and an intracellular signaling domain. In embodiments, the antigen binding domain binds a tumor antigen selected from a group consisting of: TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LACE-1a, MAGE-A1, legumain, HPV E6, E7, MAGE Al, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1. In embodiments, the intracellular signaling domain comprises a co-stimulatory signaling domain, or a primary signaling domain and a co-stimulatory signaling domain, wherein the co-stimulatory signaling domain comprises a functional signaling domain of a protein selected from the group consisting of CD27, CD28, 4-1BB(CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D.

In embodiments, the polynucleotide is an mRNA, which is not integrated into the genome of the modified cell. In embodiments, the polynucleotide is associated with an oxygen-sensitive polypeptide domain. In embodiments, the oxygen-sensitive polypeptide domain comprises Hif1a VHL binding domain.

In embodiments, the polynucleotide is regulated by a promoter comprising a binding site for a transcription modulator that modulates the expression and/or secretion of the therapeutic agent in the cell. In embodiments, the transcription modulator is or includes Hif1a, NFAT, FOXP3, and/or NFkB.

In embodiments, the modified cells comprise a polynucleotide encoding a dominant negative form of an inhibitory immune checkpoint molecule or a receptor thereof. In embodiments, the inhibitory immune checkpoint molecule is selected from the group consisting of programmed death 1 (PD-1), cytotoxic T lymphocyte antigen-4 (CTLA- 4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIRD, natural killer cell receptor 2B4 (2B4), and CD 160.

In embodiments, the modified cell has a reduced expression of endogenous TRAC gene. In embodiments, the modified cells include a nucleic acid encoding hTERT or a nucleic acid encoding SV40LT, or a combination thereof.

In embodiments, the modified cells comprise an exogenous polynucleotide encoding a therapeutic agent, and the therapeutic agent comprises at least one of IL-1P, IL-2, IL-4, IL-5, IL-6, IL-7, IL-8, IL-10, IL-12, IL-13, IL-15, IL-17, IL-1Ra, IL-2R, IFNγ, MIP-In, MIP-IP, MCP-1, TNFα, GM-CSF, GCSF, CXCL9, CXCL10, CXCR factors, VEGF, RANTES, EOTAXIN, EGF, HGF, FGF-P, and ferritin. In embodiments, the therapeutic agent comprises at least one of IL-6, IL-12, IL-7, and IFNγ.

CD47 (Cluster of Differentiation 47), also known as integrin associated protein (IAP), is a transmembrane protein that in humans is encoded by the CD47 gene. CD47 belongs to the immunoglobulin superfamily and partners with membrane integrins and also binds the ligands thrombospondin-1 (TSP-1) and signal-regulatory protein alpha (SIRPα). CD47 interacts with SIRPα, an inhibitory transmembrane receptor present on myeloid cells. The CD47/SIRPα interaction leads to bidirectional signaling, resulting in different cell-to-cell responses, including inhibition of phagocytosis, stimulation of cell-cell fusion, and T-cell activation. It has been reported that macrophages have the potential to be a powerful cellular immunotherapeutic agent in this setting if properly activated and redirected. Thus, there is a need to develop immunotherapy using lymphocytes other than T cells while reducing immunosuppression associated with these lymphocytes, such as a monocyte, macrophage, or dendritic cell.

Embodiments relate to a modified cell comprising: a first nucleic acid that encodes modified signal regulatory protein-alpha (SIRPα) that interferes with an interaction between SIRPα and CD47, the modified SIRPα lacking a functional SIRPα intracellular domain; and a second nucleic acid that encodes a chimeric antigen receptor (CAR) comprising an extracellular domain, a transmembrane domain, and an intracellular domain, and wherein the modified SIRPα and the CAR are expressed as gene products that are separate polypeptides. Embodiments relate to a modified cell comprising: a CAR comprising an extracellular domain, a transmembrane domain, and an intracellular domain; and modified SIRPα that interferes with an interaction between SIRPα and CD47, the modified SIRPα lacking a functional SIRPα intracellular domain. Embodiments relate to a pharmaceutical composition comprising the modified cell and a pharmaceutically acceptable carrier. Embodiments relate to the use of the modified cell in the manufacture of a medicament for the treatment of a tumor or cancer in a subject in need thereof. Embodiments relate to a method of treating a tumor in a subject, comprising administering to the subject a therapeutically effective amount of a pharmaceutical composition comprising the modified cell.

Embodiments relate to a method of modifying a cell, the method comprising: introducing a CAR into the cell, and wherein the cell comprises a modified SIRPα that interferes with an interaction between SIRPα and CD47, the modified SIRPα lacking a functional SIRPα intracellular domain.

In embodiments, the cell is a monocyte, macrophage, or dendritic cell. In embodiments, the cell is a macrophage. In embodiments, the intracellular domain of the modified SIRPα comprises or consist essentially of amino acid SEQ ID: 36 or 38. In embodiments, the modified SIRPα is over-expressed as compared to the wild-type receptor.

In embodiments, the extracellular domain comprises an antigen binding domain that binds an antigen comprising Epidermal growth factor receptor (EGFR), Variant III of the epidermal growth factor receptor (EGFRvIII), Human epidermal growth factor receptor 2 (HER2), Mesothelin (MSLN), Prostate-specific membrane antigen (PSMA), Carcinoembryonic antigen (CEA), Disialoganglioside 2 (GD2), Interleukin-13Ra2 (IL-13Rα2), Glypican-3 (GPC3), Carbonic anhydrase IX (CAIX), L1 cell adhesion molecule (L1-CAM), Cancer antigen 125 (CA125), Cluster of differentiation 133 (CD133), Fibroblast activation protein (FAP), Cancer/testis antigen 1B (CTAG1B), Mucin 1 (MUC1), Folate receptor-α (FR-α), CD19, FZD10, TSHR, PRLR, Muc 17, GUCY2C, CD207, CD3, CDS, B-Cell Maturation Antigen (BCMA), or CD4. In Embodiments, the intracellular domain of the CAR comprises a co-stimulatory signaling region that comprises an intracellular domain of a co-stimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, and any combination thereof.

In embodiments, the antigen binding molecule is a chimeric antigen receptor (CAR) or a T Cell Receptor (TCR).

Embodiments relate to an in vitro method for preparing modified cells. The method can include obtaining a sample of cells from the subject. For example, the sample may include T cells or T cell progenitors. The method can further include transfecting the cells with a DNA encoding at least a CAR, culturing the population of CAR cells ex vivo in a medium that selectively enhances proliferation of CAR-expressing T cells.

In embodiments, the sample is a cryopreserved sample. In embodiments, the sample of cells is from umbilical cord blood or a peripheral blood sample from the subject. In embodiments, the sample of cells is obtained by apheresis or venipuncture. In embodiments, the sample of cells is a subpopulation of T cells.

The cell surface has a CD47 signal, a signal of “don't eat me.” Almost all cancer cells have high levels of CD47 on their surface, and CD47 inhibits their ability to kill cancer cells by binding to the surface of macrophages and a protein called SIRPα. Blocking CD47 is a promising cancer treatment strategy. A protein called CD24, which is highly expressed on the surface of cancer cells, also sends a “don't eat me” signal to macrophages through the inhibitory receptor Siglec-10 (sialic acid binding Ig-like lectin 10), which is highly expressed on tumor-associated macrophages. Binding to Ig-like lectin 10 inhibits phagocytosis by macrophages. CD24 signaling usually appears to be a complementary pathway for CD47 signalings, such as in blood cancer, which appears to be very susceptible to CD47 signaling and is insensitive to CD24 signaling blockade, in contrast to other cancers, such as ovarian cancer. This allows most cancers to be attacked by blocking one of the signals. In addition, if you stop multiple “don't eat me” signals on your tumor, cancer may be more vulnerable. CD24 seems to be the most important protein of the “don't eat me” proteins.

Embodiments relate to modified cells expressing siglec-10 and/or SIRPα molecules, which bind to CD24 and CD47 molecules on the surface of cancer cells, block the binding to macrophages, and enhancing the phagocytosis of macrophages to tumors. Expression was induced using NFAT, AP-1, NFkB, and the like, and the modified cells comprises CAR-T-NFAT/AP-1/NFkB-Siglec10/SIRPα.

Modified T-cells can be derived from a stem cell. The stem cells may be adult stem cells, embryonic stem cells, more particularly non-human stem cells, cord blood stem cells, progenitor cells, bone marrow stem cells, induced pluripotent stem cells, totipotent stem cells, or hematopoietic stem cells. A modified cell can also be a dendritic cell, an NK-cell, a B-cell, or a T-cell selected from the group consisting of inflammatory T lymphocytes, cytotoxic T lymphocytes, regulatory T lymphocytes, and helper T lymphocytes. In embodiments, modified cells can be derived from the group consisting of CD4+ T lymphocytes and CD8+T lymphocytes. Prior to expansion and genetic modification of the cells described herein, a source of cells may be obtained from a subject through a variety of non-limiting methods. T cells may be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In embodiments, any number of T cell lines available and known to those skilled in the art can be used. In embodiments, modified cells may be derived from a healthy donor, from a patient diagnosed with cancer, or from a patient diagnosed with an infection. In embodiments, the modified cell is part of a mixed population of cells that present different phenotypic characteristics.

The term “stem cell” refers to any type of cells which have the capacity for self-renewal and the ability to differentiate into other kind(s) of a cell. For example, a stem cell gives rise either to two daughter stem cells (as occurs in vitro with embryonic stem cells in culture) or to one stem cell and a cell that undergoes differentiation (as occurs, e.g., in hematopoietic stem cells, which give rise to blood cells). Different categories of stem cells can be distinguished on the basis of their origin and/or on the extent of their capacity for differentiation into other types of cells. For example, a stem cell can include embryonic stem (ES) cells (i.e., pluripotent stem cells), somatic stem cells, induced pluripotent stem cells, and any other types of stem cells.

The pluripotent embryonic stem cells can be found in the inner cell mass of a blastocyst and have high innate capacity for differentiation. For example, pluripotent embryonic stem cells can have the potential to form any type of cell in the body. When grown in vitro for long periods of time, ES cells maintain pluripotency: progeny cells retain the potential for multilineage differentiation.

Somatic stem cells can include fetal stem cells (from the fetus) and adult stem cells (found in various tissues, such as bone marrow). These cells have been regarded as having a capacity for differentiation that is lower than that of the pluripotent ES cells—with the capacity of fetal stem cells being greater than that of adult stem cells. Somatic stem cells apparently differentiate into only a limited number of different types of cells and have been described as multipotent. The “tissue-specific stem cells” normally give rise to only one type of cell. For example, ES cells can be differentiated into blood stem cells (e.g., hematopoietic stem cells (HSCs)), which may be further differentiated into various blood cells (e.g., red blood cells, platelets, white blood cells, etc.).

Induced pluripotent stem cells (i.e., iPS cells or iPSCs) can include pluripotent stem cells artificially derived from a non-pluripotent cell (e.g., an adult somatic cell) by inducing expression of specific genes. Induced pluripotent stem cells are similar to natural pluripotent stem cells, such as embryonic stem (ES) cells, in many aspects, such as the expression of certain stem cell genes and proteins, chromatin methylation patterns, doubling time, embryoid body formation, teratoma formation, viable chimera formation, and potency and differentiability. Induced pluripotent cells can be made from adult stomach, liver, skin cells, and blood cells.

Embodiments relate to a nucleic acid encoding one or more therapeutic agents that inhibit CD47 and/or CD24 signaling pathways. Embodiments relate to a modified cell comprising a nucleic acid encoding one or more therapeutic agents that inhibit CD47 and/or CD24 signaling pathways. Embodiments relate to a method for enhancing T cell response (e.g., expansion and/or activation) in vitro and/or in vivo, the method comprising: introducing a nucleic acid encoding one or more therapeutic agents that inhibit CD47 and/or CD24 signaling pathway to obtain a modified cell; culturing the modified cell to obtain a population of modified cells; and contacting the population of modified cells with cells including an antigen, wherein the modified cells enhance the T cell response as compared to the cell contacted with modified cells not comprising the nucleic acid.

In embodiments, the polynucleotide can integrate into the genome of the modified cell, and progenies of the modified cell will express the polynucleotide, resulting in stably transfected modified cells. In embodiments, the modified cell express the polynucleotide encoding the CAR, but the polynucleotide does not integrate into the genome of the modified cell such that the modified cell expresses the transiently transfected polynucleotide for a finite period of time (e.g., several days), after which the polynucleotide is lost through cell division or other cellular processes. For example, the polynucleotide is present in the modified cell in a recombinant DNA construct, in an mRNA, or in a viral vector, and/or the polynucleotide is an mRNA, which is not integrated into the genome of the modified cell.

In embodiments, the one or more therapeutic agents comprise one or more recombinant proteins. In embodiments, the one or more recombinant proteins comprise secretable/soluble proteins. In embodiments, the proteins can be in a dominant negative form (e.g., SEQ NO: 78 and 79). In embodiments, the one or more recombinant proteins comprise a dominant negative form of a corresponding protein.

As defined herein, a secretable or soluble protein can be used interchangeably and refer to receptor or ligand polypeptides that are not bound to a cell membrane. Soluble proteins are most commonly ligand-binding polypeptides that lack transmembrane and cytoplasmic domains. Soluble proteins can include additional amino acid residues, such as affinity tags that provide for purification of the polypeptide or provide sites for attachment of the polypeptide to a substrate or immunoglobulin constant region. Many cell-surface receptors or ligands have naturally occurring soluble counterparts that are produced by proteolysis. Soluble polypeptides are said to be substantially free of transmembrane and intracellular polypeptide segments when they lack sufficient portions of these segments to provide membrane anchoring or signal transduction, respectively.

In embodiments, the one or more recombinant proteins comprise a recombinant protein of Siglec10 and/or a recombinant protein of SIRPα. In embodiments, the one or more therapeutic agents are or comprise a ligand for CD47 and/or a ligand for CD24. The sequences of the recombination proteins are found in Table 2. In embodiments, the one or more recombinant proteins are associated with an oxygen-sensitive polypeptide domain.

In embodiments, the nucleic acid encoding the one or more recombinant proteins comprise a promoter comprising a binding site for a transcription modulator that modulates the expression and/or secretion of the therapeutic agent in the cell. In embodiments, the transcription modulator is or includes Hif1a, NFAT, FOXP3, AP-1, Notch, and/or NFkB.

In embodiments, the nucleic acid encodes a binding molecule (e.g., CAR or TCR). In embodiments, the modified cell comprises a binding molecule (e.g., CAR or TCR). In embodiments, the nucleic acid is present in the modified cell in a recombinant DNA construct, in an mRNA, or in a viral vector. In embodiments, the nucleic acid is an mRNA, which is not integrated into the genome of the modified cell.

Embodiments relate to a method or use of polynucleotides. The method or use includes: providing a viral particle (e.g., adeno-associated virus (AAV), lentivirus or their variants) comprising a vector genome, the vector genome comprising the polynucleotide, wherein the polynucleotide is operably linked to an expression control element conferring transcription of the polynucleotide, and administering an amount of the viral particle to the subject such that the polynucleotide is expressed in the subject. In embodiments, the AAV preparation can include AAV vector particles, empty capsids, and host cell impurities, thereby providing an AAV product substantially free of AAV empty capsids. More information on the administration and preparation of the viral particles can be found at the US Patent NO: 9840719 and Milani et al., Sci. Transl. Med. 11, eaav7325 (2019) 22 May 2019, which are incorporated herein by reference.

In embodiments, the nucleic acid comprises a sequence encoding a cleavable peptide (e.g., 2A or IRES), which is disposed between at least two cytokines.

In embodiments, the one or more therapeutic agents are or comprise a secretable/soluble protein of Siglec10, a secretable/soluble protein of SIRPα, a dominant negative form of Siglec10, and/or a dominant negative form of SIRPα.

In embodiments, the nucleic acid encodes a CAR, or the modified cell comprises a CAR. In embodiments, the CAR comprises an extracellular domain, a transmembrane domain, and an intracellular domain, the extracellular domain binding an antigen. In embodiments, the CAR binds tMUC 1, PRLR, CLCA1, MUC12, GUCY2C, GPR35, CR1L, MUC 17, TMPRSS11B, MUC21, TMPRSS11E, CD207, SLC30A8, CFC1, SLC12A3, SSTR1, GPR27, FZD10, TSHR, SIGLEC15, SLC6A3, KISS1R, QRFPR, GPR119, CLDN6, UPK2, ADAM12, SLC45A3, ACPP, MUC21, MUC16, MS4A12, ALPP, CEA, EphA2, FAP, GPC3, IL-13Ra2, Mesothelin, PSMA, ROR1, VEGFR-II, GD2, FR-α, ErbB2, EpCAM, EGFRvIII, PSCA, or EGFR. In embodiments, the intracellular domain comprises a co-stimulatory domain that comprises an intracellular domain of a co-stimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, and one combination thereof. In embodiments, the antigen is Epidermal growth factor receptor (EGFR), Variant III of the epidermal growth factor receptor (EGFRvIII), Human epidermal growth factor receptor 2 (HER2), Mesothelin (MSLN), Prostate-specific membrane antigen (PSMA), Carcinoembryonic antigen (CEA), Disialoganglioside 2 (GD2), Interleukin-13Ra2 (IL-13Ra2), Glypican-3 (GPC3), Carbonic anhydrase IX (CAIX), L1 cell adhesion molecule (L1-CAM), Cancer antigen 125 (CA125), Cluster of differentiation 133 (CD133), Fibroblast activation protein (FAP), Cancer/testis antigen 1B (CTAG1B), Mucin 1 (MUC1), Folate receptor-α (FR-α), CD19, FZD10, TSHR, PRLR, Muc 17, GUCY2C, CD207, CD3, CD5, B-Cell Maturation Antigen (BCMA), or CD4.

Embodiments relate to a composition for treating a subject having cancer, the composition comprising a first population of cells and a second population of cells, wherein the first population of cells comprising a nucleic acid encoding a binding molecule binding a solid tumor antigen, a second population of cells comprising the nucleic acid described above encoding the cleavable peptide.

Embodiments relate to a composition for treating a subject having cancer, the composition comprising a first population of cells and a second population of cells, wherein the first population of cells comprising a nucleic acid encoding a binding molecule binding a white blood antigen, a second population of cells expressing and/or secreting the one or more therapeutic agents of described herein.

Systemic autoimmune diseases (SAD) can be life-threatening. For example, 10-year survival rate of one type of SAD, Systemic Lupus Erythematosus (SLE), is 89%. On the other hand, patients with Multiple Sclerosis (MS) have shortened life expectancy by 7 to 14 years compared to the average population. Furthermore, the quality of life is generally compromised from disease symptoms and flares and routine avoidance of environmental triggers. Therapeutic measures for these diseases are usually supportive treatment due to their incurable nature. Conventionally, systemic administration of immunosuppressants is used to manage disease severity and decrease relapse rate, but the long-term use of immune-suppressive drugs can result in various side effects, including fatigue, allergies, shingles, kidney problems, and heart problems.

Recently, B lymphocyte depleting therapy has been shown to effectively suppress SLE and MS activity measures, including developing new enhancing lesions and relapse rates. In addition, unlike conventional systemic immunosuppressive drugs, which include those that excessively down-regulate a vast array of immune activities, monoclonal antibodies targeting CD19, CD20, B cell maturation antigen (BCMA), or BAFF-R specifically deplete B lymphocyte-mediated immune responses, therefore minimizing possible side effects of systemic immunosuppression. However, the therapeutic regime using monoclonal antibodies usually requires weekly intravenous (IV) administration for over one month, and the average half-life after a complete infusion is approximately 21 days (for Rituximab®). Thus, the time-to-effect only begins approximately six weeks after the primary infusion, and the full effect usually does not take place until the third month. Moreover, disease symptoms can resurface as soon as nine months post-infusion.

Autoimmune disorders occur when autoreactive immune cells are induced to activate their responses against healthy self-tissues. Autoimmune disorders affect one percent of the world population, and represent one of the top 10 leading causes of death. The major histocompatibility complex (MHC) is a principal susceptibility locus for many human autoimmune diseases, in which self-tissue antigens providing targets for pathogenic lymphocytes are bound to HLA molecules encoded by disease-associated alleles.

There are more than 80 different autoimmune diseases. Examples of autoimmune diseases include type 1 diabetes, rheumatoid arthritis, psoriasis/psoriatic arthritis, multiple sclerosis, systemic lupus erythematosus (SLE), inflammatory bowel disease, Addison's disease, graves' disease, sjögren's syndrome, Hashimoto's thyroiditis, myasthenia gravis, autoimmune vasculitis, pernicious anemia, and celiac disease. For example, in Type 1 diabetes (T1D), the pancreas produces the hormone insulin, which helps regulate blood sugar levels. In type 1 diabetes mellitus, the immune system attacks and destroys insulin-producing cells in the pancreas, and high blood sugar results can lead to damage in the blood vessels and organs like the heart, kidneys, eyes, and nerves. In rheumatoid arthritis (RA), the immune system attacks the joints. This attack causes redness, warmth, soreness, and stiffness in the joints. Unlike osteoarthritis, which commonly affects people as they get older, RA can start in patients as early as those in their 30s or sooner.

Skin cells normally grow and then shed when they're no longer needed. Psoriasis causes skin cells to multiply quickly. The extra cells build up and form inflamed, red patches, commonly with silver-white scales of plaque on the skin. Up to 30 percent of people with psoriasis also develop swelling, stiffness, and pain in their joints. This form of the psoriasis is called psoriatic arthritis.

Multiple sclerosis (MS) damages the myelin sheath, the protective coating surrounding nerve cells in a patient's central nervous system. Damage to the myelin sheath slows the transmission speed of messages between the patient's brain and spinal cord and the rest of patient's body. This damage can lead to symptoms like numbness, weakness, balance issues, and trouble walking. The disease comes in several forms that progress at different rates. According to a 2012 study, about 50 percent of people with MS need help walking within 15 years after the disease starts. With SLE, although doctors in the 1800s first described lupus as a skin disease because of the rash it commonly produces, the systemic form, which is most the common, actually affects many organs, including the joints, kidneys, brain, and heart. Joint pain, fatigue, and rashes are among the most common symptoms. Inflammatory bowel disease (IBD) is a term used to describe conditions that cause inflammation in the intestinal wall lining. Each type of IBD affects a different part of the GI tract. Crohn's disease can inflame any part of the GI tract, from the mouth to the anus. Ulcerative colitis affects only the lining of the large intestine (colon) and rectum. Addison's disease affects the adrenal glands, which produce the hormones cortisol and aldosterone as well as androgen hormones. Low cortisol level affects how the body uses and stores carbohydrates and sugar (glucose). Aldosterone deficiency leads to sodium loss and excess potassium in the bloodstream. Symptoms include weakness, fatigue, weight loss, and low blood sugar.

Graves' disease attacks the thyroid gland in the neck, causing it to produce too much of thyroid hormones. Thyroid hormones control the body's energy usage, known as metabolism. An over production of these hormones accelerates the body's metabolism causing symptoms like nervousness, a fast heartbeat, heat intolerance, and weight loss. One potential symptom of this disease is bulging eyes, called exophthalmos. It can occur as a part of what is called Graves' ophthalmopathy, which occurs in around 30 percent of those who have Graves' disease. Sjögren's syndrome is a condition in which immune cells attacks the glands that provide lubrication to the eyes and mouth. The hallmark symptoms of Sjögren's syndrome are dry eyes and dry mouth, but it may also affect the joints or skin. In Hashimoto's thyroiditis, thyroid hormone production slows to a deficiency. Symptoms include weight gain, sensitivity to cold, fatigue, hair loss, and thyroid swelling (goiter). Myasthenia gravis affects nerve impulses that help the brain control the muscles impairing the communication between nerves and muscles. The most common symptom is muscle weakness that gets worse with activity and improves with rest. Often muscles that control eye movements, eyelid opening, swallowing, and facial movements are affected.

Autoimmune vasculitis is a disease in which the immune system attacks the blood vessels causing inflammation that results in the narrowing of the arteries and veins, restricting blood to flow. Pernicious anemia is a condition caused by a lack of intrinsic factor, a protein made by the cells of the stomach lining that is needed by the small intestine to absorb vitamin B12 from food. A lack of the protein leads to vitamin B12 deficiency which induces alterations in DNA synthesis in an individual. Moreover, without a sufficient amount of vitamin B12, an individual develops anemia. Pernicious anemia is more common in older adults and affects 0.1 percent of people in general, but nearly 2 percent of people over age 60.

Although celiac disease is not an autoimmune disease, people with celiac disease can't eat foods containing gluten, a protein found in wheat, rye, and other grain products, which triggers an immune response in the gastrointestinal tract. Over time, the immune response can damage the intestinal lining and prevent it from absorbing the required nutrients. The damage to the intestinal causes diarrhea, fatigue, weight loss, bloating, an anemia, and other complications. A 2015 study found that celiac disease affects about 1 percent of people in the United States. There is no cure for celiac disease but the condition can be managed by following a strict gluten free diet.

Embodiments relate to the treatment of autoimmune disease with immunotherapy. Embodiments include methods and systems for the use of chimeric antigen receptor-modified cells to treat autoimmune diseases. For example, the method includes administering a pharmaceutically effective amount of a population of T cells of the subject modified to express a CAR that binds to an antigen associated with B cells. In embodiments, the autoimmune disease is an autoimmune disease associated with B cell proliferation and immunoglobulin secretion. In embodiments, the autoimmune disease is SLE, RA, T1D, Sjögren's syndrome, or MS. In embodiments, the antigen is BAFF, APRIL, CD19, CD20, CD22, a B cell receptor (BCR), BCMA, or CD79. In embodiments, the antigen is CD19, CD20, or BAFF. In embodiments, the pharmaceutically effective amount of a population of T cells is about 104 to 109 cells per kg body weight of the human patient. In embodiments, the pharmaceutically effective amount of a population of T cells is about 105 to 106 cells per kg body weight of the human patient. In embodiments, the pharmaceutically effective amount of a population of T cells is about 106 to 107 cells per kg body weight of the human patient. In embodiments, the pharmaceutically effective amount of a population of T cells is about 104 to 105 cells per kg body weight of the human patient. In embodiments, the pharmaceutically effective amount of a population of T cells is about 107 to 108 cells per kg body weight of the human patient. In embodiments, the pharmaceutically effective amount of a population of T cells is about 108 to 109 cells per kg body weight of the human patient.

Embodiments relate to compositions and methods for treating autoimmune diseases. For example, a composition comprises a population of modified cells effective for expanding and/or maintaining the modified cells in a patient, wherein the population of modified cells comprises at least two different modified cells: a first modified cell comprising a first antigen binding domain for expanding, maintaining the modified cells, and/or killing B cells; and a second modified cell comprising a second antigen binding domain for killing a target cell, such as a T cell causing autoimmune disease. In embodiments, the modified cells are modified T cells. In embodiments, the at least two different modified cells include two different modified T cells, two different modified immune cells, or a combination thereof, and/or wherein the modified immune cells include modified T cells, DC cells, and/or macrophages.

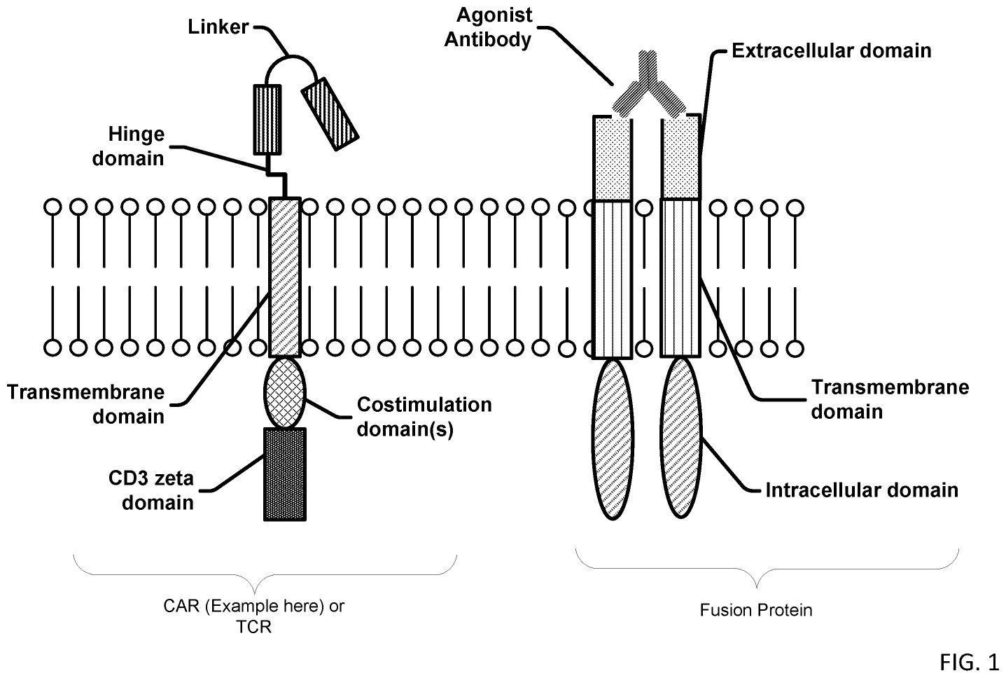

HLA-peptide complex (HPC) refers to lymphocyte epitopes or antigens restricted by the HLA complex. For example, T cell epitopes are presented on the surface of an antigen-presenting cell, where they are bound to MHC molecules. In humans, professional antigen-presenting cells are specialized to present MHC class II peptides, whereas most nucleated somatic cells present MHC class I peptides. T cell epitopes presented by MHC class I molecules can typically include peptides between 8 and 11 amino acids in length. In contrast, MHC class II molecules present longer peptides, 13-17 amino acids in length, and non-classical MHC molecules also present non-peptidic epitopes such as glycolipids. In embodiments, an HPC can include α1, α2, and α3 domains, β-macroglobulin, and an antigen peptide (See HLA-Peptide soluble in FIG. 1). Methods for identifying HPC can be found at Zhao L, Zhang M, Cong H. Advances in the study of HLA-restricted epitope vaccines. Hum Vaccin Immunother. 2013 December;9(12):2566-77. doi: 10.4161/hv.26088. Epub 2013 Aug. 16. PMID: 23955319; PMCID: PMC4162067, which is incorporated in its entirety.

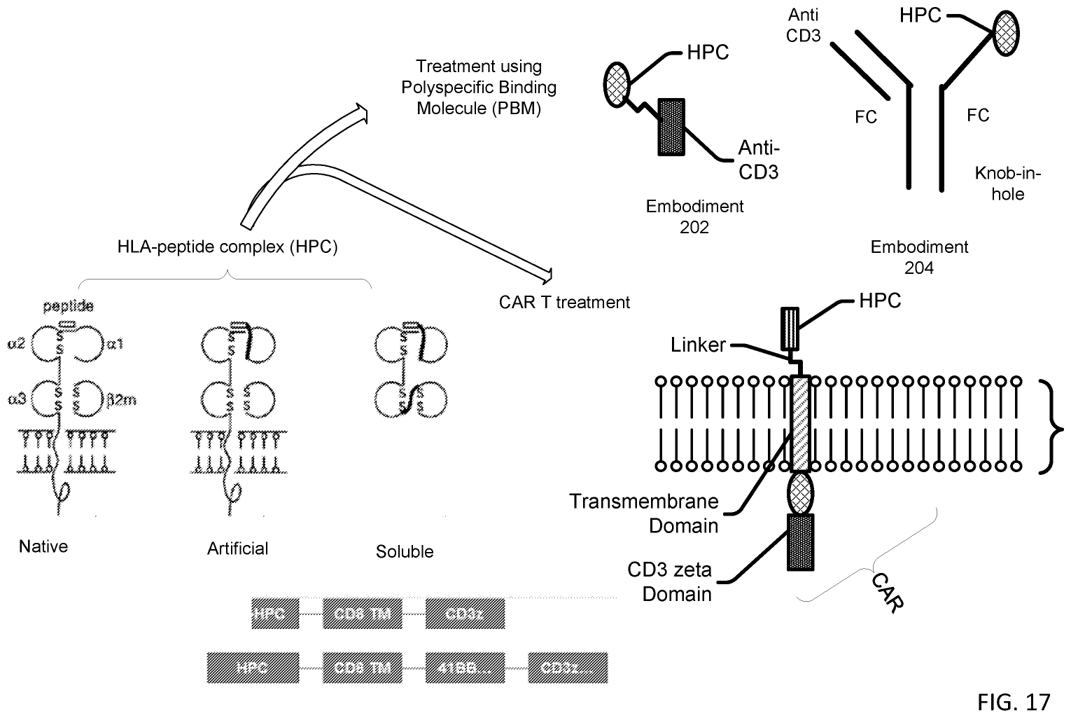

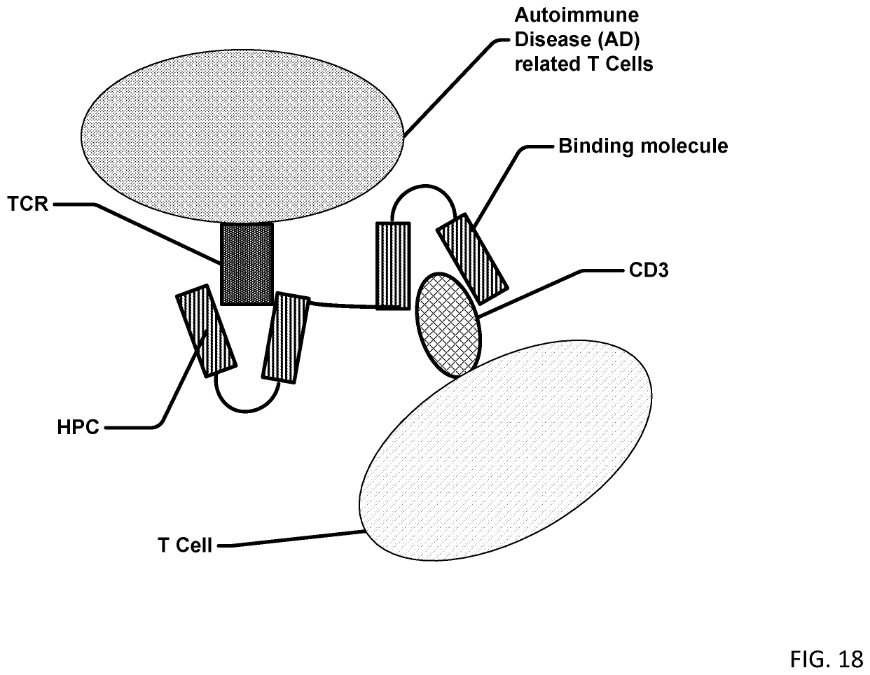

ADHPC refers to an HPC associated with T cell-mediated autoimmune diseases. Autoimmune diseases include T cell-mediated autoimmune diseases (e.g., type 1 diabetes mellitus in infancy, hypothyroidism, and Addison's disease) caused by the attack on these organs by the patient's immune cells. In embodiments, ADHPC mediates T cell-mediated autoimmune diseases. Examples of ADHPC may be found at FIGS. 17 and 18, as well as SEQ ID Nos: 127 and 128.

Embodiments of the present disclosure include fusion proteins and uses for treating autoimmune diseases thereof. For example, a fusion protein (e.g., polyspecific binding molecule (e.g., polyspecific antibody)) comprises a first antigen binding domain targeting a receptor of an antigen of T cells; and a second antigen binding domain targeting a receptor of an ADHPC. ADHPC refers to an HPC associated with T cell-mediated autoimmune diseases. Autoimmune diseases include T cell-mediated autoimmune diseases (e.g., type 1 diabetes mellitus in infancy, hypothyroidism, and Addison's disease) caused by the attack on these organs by the patient's immune cells. In embodiments, ADHPC mediates T cell-mediated autoimmune diseases. A method for treating a subject with an autoimmune disease comprises administering an effective amount of the composition comprising the fusion protein. In embodiments, the method further comprises administering an effective amount of the composition comprising an additional fusion protein target B cells (e.g., BITE® CD19-CD3).

Embodiments include the use of abnormal T cells to recognize a certain target to kill normal cells or activate themselves through the target. In embodiments, CARs and/or bispecific binding molecules are designed to recognize and remove abnormal immune cells, thereby removing the source of autoimmune diseases.

Embodiments describe methods and compositions of HPC-based immunotherapies. Embodiments relate to a polynucleotide encoding an antigen binding molecule (e.g., a CAR and a polyspecific binding molecule (for example a bispecific binding molecule, such as a BITE)) that comprises HPC. Embodiments relate to a composition comprising a first CAR T cell or a bispecific binding molecule targeting a B cell antigen and a second CAR cell or a bispecific binding molecule comprising HPC as a binding domain. Embodiments relate to a modified cell comprising the antigen binding molecule and treating autoimmune diseases using the compositions.

As used herein, a polyspecific binding molecule refers to a molecule capable of binding more than one target. Examples of the polyspecific binding molecule include a bispecific binding molecule and a triple-specific binding molecule. Examples of the binding molecule may include an antibody, a receptor (e.g., TCR), a ligand, an agonist, a cytokine, etc. The binding molecules can be connected via various compositions such as a linker, a nanoparticle, a bead, a surface, and the like. More information on bispecific and tri-specific binding molecules can be found at Runcie et al. Molecular Medicine (2018) 24:50 and Oates J, Hassan N J, Jakobsen B K. ImmTACs for targeted cancer therapy: Why, what, how, and which. Mol Immunol. 2015;67(2 Pt A):67-74. doi:10.1016/j.molimm.2015.01.024, which are incorporated by reference in their entirety. In embodiments, a polyspecific binding molecule may include an antibody binding a T cell, a linker, and an antibody binding a solid tumor antigen. In embodiments, a polyspecific binding molecule includes a TCR binding a TCR antigen (e.g., HLA-restricted peptide antigen) and an antibody binding a T cell. Examples of the TCR antigen include CEA, gp100, MART-1, p53, MAGE-A314, and NY-ESO-1. In embodiments, a bispecific binding molecule comprises a first and a second binding domain, wherein the first binding domain binds to a solid tumor antigen, and the second binding domain binds, for example, the T cell CD3 receptor complex or CD28.

HLAA2-P1-CAR kills T cells that specifically recognize HLAA2-P1 in vitro. The CAR includes an scFv specifically that recognizes the MAGEA4 peptide presented by HLA-A2. Vectors encoding the scFv is packaged into a lentivirus to infect Jurkat cells (T lymphoma cell line). The HLAA2-peptide was constructed as an extracellular, intracellular connection with 41BB, CD28. CD40, CD27 or other co-stimulatory signals and CD3z, CD3e, or other activation signals, to assemble into a CART that can specifically recognize and kill the Jurkat cells. Accordingly, CAR T cells can specifically recognize the above Jurkat cells, activate, release cytokines, and expand in a large amount while effectively killing the Jurkat cells.

Type 1 diabetes is known to specifically kill HLA-A2 pancreatic β cells presenting defective ribosomal products (DRIPs) by T cells. In embodiments, an scFv targeting HLA-A2-DRIPs may be generated, and a vector encoding the scFv may be transferred to Jurkat cells. These Jurkat cells may be used for testing effacacy of CAR T cells for treating type 1 diabetes. The binding domain of the CAR includes HLA-A2-DRIPs, which bind the scFv expressed on the Jurkat cells. In embodiments, cytotoxic T lymphocytes (CTLs) may be collected from a subject having type 1 diabetes and then used to teat effacacy of CAR T cells for treating type 1 diabetes. In embodiments, peripheral blood mononuclear cells (PBMCs) from a subject having type 1 diabetes may be cultured with HLA-A2-DRIPs and then used to teat effacacy of CAR T cells for treating type 1 diabetes. Embodiments relate to a bispecific chimeric antigen receptor comprising: a first antigen binding domain, a second antigen binding domain, a cytoplasmic domain, and transmembrane domain, wherein the first antigen binding domain recognizes a first antigen, and the second antigen binding domain recognizes a second antigen, and the first antigen is different from the second antigen. In embodiments, the first antigen is CLDN18.2, and the second antigen is GUCY2C. In embodiments, the bispecific CAR comprises amino acid SEQ ID NO: 128 or 129.

In embodiments, the two different antigen binding domains can be on the same binding molecule, for example on a bispecific CAR, and encoded by a single nucleic acid. In embodiments, the bispecific CAR can have two different scFv molecules joined together by linkers. Examples of bispecific CARs are provided in Table 2.

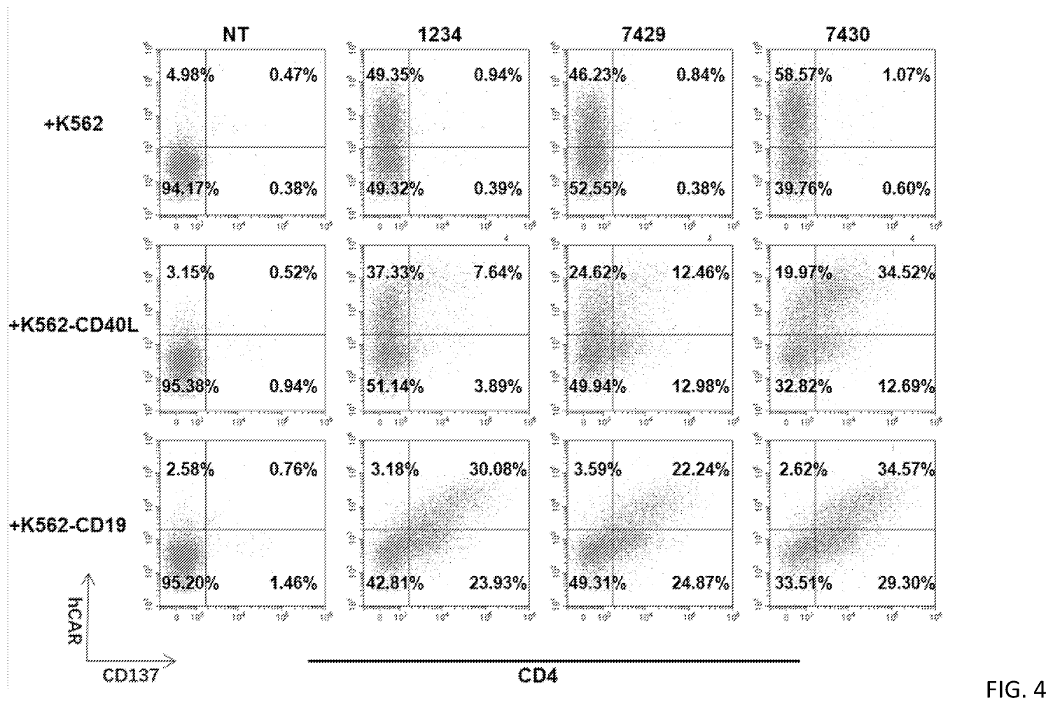

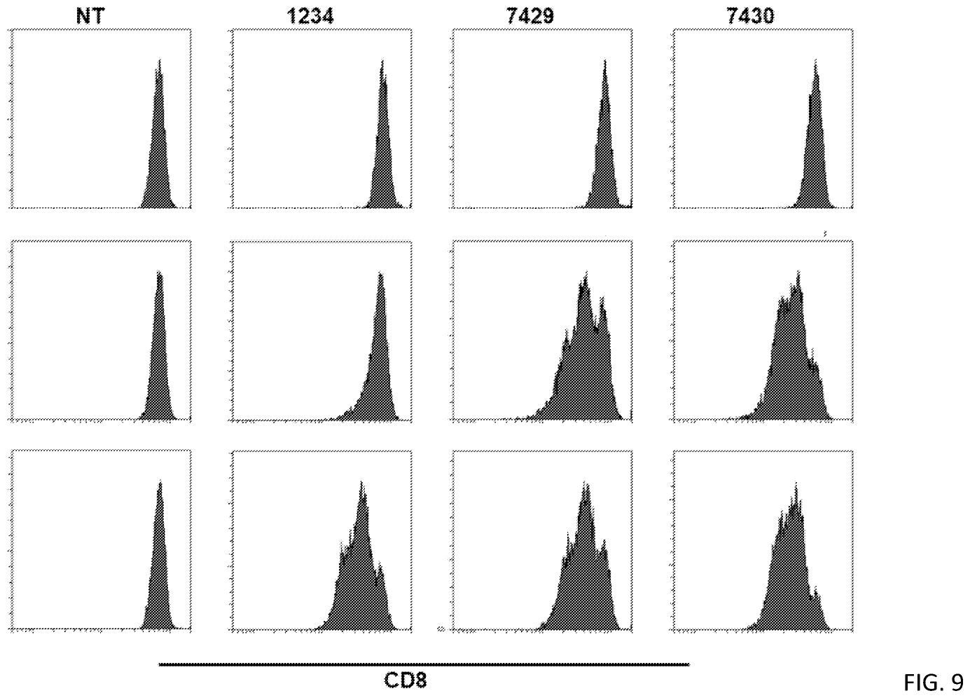

Examples of a bispecific CAR are shown in FIGS. 4 and 9. As shown in FIG. 9, a bispecific CAR (or tandem CAR (tanCAR)) can include two binding domains: scFv1 and scFv2. In embodiments, scFv1 binds an antigen of a white blood cell (e.g., CD19), and scFv2 binds a solid tumor antigen (e.g., tMUC1). In embodiments, scFv1 binds a solid tumor antigen, and scFv2 binds another solid tumor antigen (e.g., tMUC1 and CLDN 18.2). Claudin18.2 (CLDN 18.2) is a stomach-specific isoform of Claudin-18. CLDN 18.2 is highly expressed in gastric and pancreatic adenocarcinoma. In embodiments, scFv1 binds an antigen expressed on tumor cells but not on normal tissues (e.g., tMUC1); scFv2 binds an antigen expressed on nonessential tissues associated with solid tumor; and the killing of normal cells of the tissue does not cause a life-threatening event (e.g., complications) to the subject (e.g., TSHR, GUCY2C). Examples of the nonessential tissues include organs such as prostate, breast, or melanocyte. In embodiments, scFv1 and scFv2 bind to different antigens that are expressed on the same nonessential tissue (e.g., ACPP and SLC45A3 for Prostate cancer, and SIGLEC15 and UPK2 for Urothelial cancer).

Embodiments relate to a bispecific chimeric antigen receptor, comprising: a first antigen binding domain, a second antigen binding domain, a cytoplasmic domain, and transmembrane domain, wherein the first antigen binding domain recognizes a first antigen, and the second antigen binding domain recognize a second antigen, and the first antigen is different from the second antigen. Embodiments relate to a cell comprising the bispecific CAR. Embodiments relate to a nucleic acid encoding the bispecific CAR. Embodiments relate to a method of enhancing T cell response, enhancing treatment of cancer, treating cancer in a subject, treating a subject having a tumor, or inhibiting the growth of a tumor, the method comprising: administering an effective amount of modified cell comprising a CAR. In embodiments, the subject has cancer. Embodiments relate to a method of enhancing treatment of tumors associated with one or more organs of the digestive tract, the method comprising: administering an effective amount of cells, wherein the T cell response thereof is enhanced as compared to administering an effective amount of cells comprising a CAR comprising one of scFv binding CLDN18.2 or GUCY2C. In embodiments, the first antigen and the second antigen are not expressed on the same cell.

In embodiments, the first antigen is an antigen of a blood component, and the second antigen is an antigen of a solid tumor. In embodiments, the first antigen is CLDN18.2, and the second antigen is GUCY2C. In embodiments, the bispecific CAR comprises the SEQ ID NO: 128 or 129.

In embodiments, the first binding domain binds an antigen of nonessential tissues, and the second binding domain binds an antigen of tumor tissue. In embodiments, the first binding domain binds TSHR or GUCY2C. In embodiments, the second binding domain binds tMUC1, MAGE-E1, or Epithelial tumor antigen (ETA).

In embodiments, the first binding domain binds a tissue specific antigen, and the second binding domain binds an antigen expressed on more than one tissue. In embodiments, the first binding domain binds TSHR or PRLR. In embodiments, the second binding domain binds tMUC1, MAG-E1, or ETA.

In embodiments, the first binding domain binds an antigen of normal tissue, and the second binding domain binds an antigen expressed on tumor tissue. In embodiments, the first binding domain binds ACPP, TSHR, GUCY2C, UPK2, CLDN18.2, PSMA, DPEP3, CXCR5, B7-H3, MUC16, SIGLEC-15, CLDN6, Muc17, PRLR, or FZD10. In embodiments, the second binding domain binds tMUC1, MAG-E1, or ETA.

In embodiments, the first binding domain binds an antigen that is expressed on non-malignant cells, and the second binding domain binds an antigen that is expressed on tumor cells and not on corresponding non-malignant cells.

More information related to CAR and its uses for treating cancer can be found in PCT Patent Publications WO2020106843 and WO2019140100 and PCT Patent Application NO: PCT/US20/13099, which are incorporated herein by reference in their entirety.

The present disclosure is further described by reference to the following exemplary embodiments and examples. These exemplary embodiments and examples are provided for purposes of illustration only and are not intended to be limiting unless otherwise specified. Thus, the present disclosure should in no way be construed as being limited to the following exemplary embodiments and examples, but rather, should be construed to encompass any and all variations which become evident as a result of the teaching provided herein.

### EXEMPLARY EMBODIMENTS

The following are exemplary embodiments: 1. A fusion protein comprising an extracellular domain, a transmembrane domain, and an intracellular domain, the extracellular domain derived from a first molecule, the intracellular domain derived from a second molecule, the first molecule different from the second molecule., wherein the first molecule is a co-stimulatory domain, and the second molecule is a cytokine receptor.

2. A fusion protein comprising an extracellular domain, a transmembrane domain, and an intracellular domain, the extracellular domain derived from a first molecule, the intracellular domain derived from a second molecule, the first molecule different from the second molecule.

3. An isolated nucleic acid encoding the fusion of embodiments 1 or 2.

4. A modified cell comprising the isolated nucleic acid of embodiment 3 or the fusion protein of embodiments 1 or 2.

5. A pharmaceutical composition comprising a population of the cells of embodiment 4.

6. A method of cause T cell response in a subject in need thereof and/or treating a tumor of the subject, the method comprising administering an effective amount of the composition of embodiment 5 to the subject.

7. The method of embodiment 6, further comprising: administering an effective amount of an agonist antibody binding the extracellular domain.

8. The method of embodiment 7, wherein the binding of the extracellular domain with the agnostic antibody causes recruitment of multiple first molecules into a receptor oligomer where signaling activation of the second molecule is triggered or enhanced.

9. The fusion protein, isolated nucleic acid, modified cell, pharmaceutical composition, or method of one of embodiments 1-8, wherein the extracellular domain binds to an agonist antibody, and wherein the agonist antibody is CDX 1140, SEA CD40, R07009789, JNJ 64457107 (ADC1013), APX 005M, Chi Lob 7/4, TRX 518, MK 4166, MK 1248, GWN 323, INCAGN01876, BMS 986156, AMG 228, Tavolimab (MED10562), PF 04518600, BMS 986178, MOXR 0916, GSK 3174998, INCAGN01949, Utomilumab (PF 05082566), Urelumab (BMS 663513), GSK 3359609, JTX 2011, Theralizumab (TAB 08).

10. The fusion protein, isolated nucleic acid, modified cell, pharmaceutical composition, or method of one of embodiments 1-8, wherein the extracellular domain is an extracellular domain of a co-stimulatory molecule.

11. The fusion protein, isolated nucleic acid, modified cell, pharmaceutical composition, or method of one of embodiments 1-8, wherein the extracellular domain is an extracellular domain of OX40, CD40, 4-1BB, GITR, ICOS, CD28, CD27, HER2, or EGFR.

12. The fusion protein, isolated nucleic acid, modified cell, pharmaceutical composition, or method of one of embodiments 1-8, wherein:
the extracellular domain is an extracellular domain of CD40, GITR, OX40, 4-1BB, ICOS, or CD28;
the co-stimulatory molecule is a co-stimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, and any combination thereof; and/or the intracellular is an intracellular domain of a cytokine receptor.

13. The fusion protein, isolated nucleic acid, modified cell, pharmaceutical composition, or method of one of embodiments 1-8, wherein the second molecule is OX40, CD40, 4-1BB, GITR, ICOS, CD28, CD27, HER2, EGFR, IL-10R, IL-12R, IL-18R1, IL-23R, GP130, or IL-15Ra.

14. The fusion protein, isolated nucleic acid, modified cell, pharmaceutical composition, or method of one of embodiments 1-8, wherein the intracellular domain is an intracellular domain of IL-12R, IL-18R1, IL-23R, GP130, or IL-15Ra.

15. The fusion protein, isolated nucleic acid, modified cell, pharmaceutical composition, or method of one of embodiments 1-14, wherein the transmembrane domain is an intracellular domain of OX40, CD40, 4-1BB, GITR, ICOS, CD28, CD27, HER2, EGFR, IL-12R, IL-18R1, IL-23R, GP130, or IL-15Ra.

16. The modified cell, pharmaceutical composition, or method of one of embodiments 4-8, wherein the modified cell is lymphocyte, leukocyte, or PBMC; or cells, NK cells, or dendritic cells.

17. The modified cell, pharmaceutical composition, or method of one of embodiments 4-16, wherein the modified cell further comprises a Chimeric antigen receptor (CAR) or a modified TCR.

18. The modified cell, pharmaceutical composition, or method of embodiment 17, wherein the TCR is modified TCR.

19. The modified cell, pharmaceutical composition, or method of embodiment 17, wherein the TCR is derived from spontaneously occurring tumor-specific T cells in patients.

20. The modified cell, pharmaceutical composition, or method of embodiment 17, wherein the TCR binds a tumor antigen.

21. The modified cell, pharmaceutical composition or method of embodiment 20, wherein the tumor antigen comprises CEA, gp100, MART-1, p53, MAGE-A3, or NY-ESO-1, or the TCR comprises TCRγ and TCRδ Chains or TCRα and TCRβ chains, or a combination thereof.

22. The modified cell, pharmaceutical composition, or method of embodiment 17, wherein the CAR comprises an extracellular domain, a transmembrane domain, and an intracellular domain, the extracellular domain binds an antigen.

23. The modified cell, pharmaceutical composition or method of embodiment 22, wherein the intracellular domain comprises a co-stimulatory signaling region that comprises an intracellular domain of a co-stimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, and any combination thereof.

24. The modified cell, pharmaceutical composition or method of embodiment 23, wherein the antigen is Epidermal growth factor receptor (EGFR), Variant III of the epidermal growth factor receptor (EGFRvIII), Human epidermal growth factor receptor 2 (HER2), Mesothelin (MSLN), Prostate-specific membrane antigen (PSMA), Carcinoembryonic antigen (CEA), Disialoganglioside 2 (GD2), Interleukin-13Ra2 (IL-13Ra2), Glypican-3 (GPC3), Carbonic anhydrase IX (CAIX), L1 cell adhesion molecule (L1-CAM), Cancer antigen 125 (CA125), Cluster of differentiation 133 (CD133), Fibroblast activation protein (FAP), Cancer/testis antigen 1B (CTAG1B), Mucin 1 (MUC1), Folate receptor-α (FR-α), CD19, FZD10, TSHR, PRLR, Muc 17, GUCY2C, CD207, CD3, CDS, B-Cell Maturation Antigen (BCMA), or CD4.

25. The modified cell, pharmaceutical composition, or method of one of embodiment 4-24, wherein the modified cell or the T cells comprise an additional CAR binding a solid tumor antigen, and the CAR binds an antigen of a white blood cell.

26. The modified cell, pharmaceutical composition, or method of one of embodiment 4-24, wherein the modified cell or the T cells comprise a dominant negative PD-1.

27. The modified cell, pharmaceutical composition, or method of one of embodiment 4-24, wherein the modified cell or the T cells comprise a modified PD-1 lacking a functional PD-1 intracellular domain.

28. The modified cell, pharmaceutical composition, or method of one of embodiment 4-27, wherein the modified cell further comprises a nucleic acid encoding therapeutic agent.

29. The modified cell, pharmaceutical composition, or method of embodiment 28, wherein the isolated nucleic acid comprises a promoter comprising a binding site for a transcription modulator that modulates the expression and/or secretion of the therapeutic agent in the cell.

30. The modified cell, pharmaceutical composition, or method of embodiment 29, wherein the transcription modulator is or includes Hif1a, NFAT, FOXP3, and/or NFkB.

31. The modified cell, pharmaceutical composition, or method of embodiment 30, wherein the promoter is responsive to the transcription modulator.

32. The modified cell, pharmaceutical composition, or method of embodiment 30, wherein the promoter is operably linked to the nucleic acid encoding the therapeutic agent such that the promoter drives expression and/or secretion of the therapeutic agent in the cell.

33. The modified cell, pharmaceutical composition, or method of embodiment 30, wherein expression of the therapeutic agent is regulated by an inducible gene expression system.

34. The modified cell, pharmaceutical composition, or method of embodiment 33, wherein the inducible gene expression system comprises or is a lac system, a tetracycline system, or a galactose system.

35. The modified cell, pharmaceutical composition, or method of embodiment 33, wherein the inducible gene expression system comprises or is a tetracycline system.

36. The modified cell, pharmaceutical composition, or method of embodiment 35, wherein the inducible gene expression system comprises or is a tetracycline on a system, and an inducer is tetracycline doxycycline, or an analog thereof.

37. The modified cell, pharmaceutical composition, or method of one of embodiments 4-36, wherein the modified cell is a T cell derived from a primary human T cell isolated from a human donor.

38. The modified cell, pharmaceutical composition, or method of embodiment 37, wherein the cell has a reduced expression of endogenous TRAC gene.

39. The modified cell, pharmaceutical composition, or method of one of embodiments 4-36, wherein the modified cell is a T cell derived from a primary human T cell isolated from a subject having cancer.

40. The modified cell, pharmaceutical composition, or method of embodiment 39, wherein the cell has a reduced expression of endogenous TRAC gene.

41. A modified cell comprising an exogenous polynucleotide encoding at least an extracellular domain of a co-stimulatory molecule.

42. A modified cell comprising an exogenous peptide comprising at least an extracellular domain of a co-stimulatory molecule.

44. The modified cell of embodiment 41 or 42, wherein the modified cell comprises the fusion protein of any preceding suitable embodiments.

44. The modified cell of any of embodiments 41-43, wherein the co-stimulatory molecule comprises CD27, CD28, 4-1BB, OX40, CD30, CD40, PD-1, ICOS, lymphocyte function-associated antigen-1 (LFA-1), CD2, CD7, LIGHT, NKG2C, B7-H3, or a combination thereof.

45. The modified cell of any of embodiments 41-44, wherein the co-stimulatory molecule comprises OX40, CD40, 4-1BB, GITR, ICOS, CD27, or CD28.

46. The modified cell of any of embodiments 41-44, wherein the co-stimulatory molecule is CD40.

47. The modified cell of any of embodiments 41-46, wherein the modified cell is derived from a lymphocyte.

48. The modified cell of any of embodiments 41-47, wherein the modified cell is derived from a T cell or NK.

49. The modified cell of any of embodiments 41-48, wherein the exogenous peptide further comprises or the exogenous polynucleotide further encodes an intracellular domain and/or a transmembrane domain of the co-stimulatory molecule.

50. The modified cell of any of embodiments 41-49, wherein the modified cell further comprises a CAR or a TCR.

51. The method cell of any of embodiments 41-49, wherein the modified cell further comprises a CAR binding a solid tumor antigen.

52. A method of expanding modified cells of any of embodiments 14-51 or enhancing T cell response of the modified cell of any of embodiments 41-51, the method comprising:
administering an effective amount of the modified cells to a subject having cancer; and
administering an effective amount of an agonist or ligand of the co-stimulatory molecule to the subject.

53. The method of embodiment 51, wherein the T cell response or cell expansion is enhanced as compared to a subject that is administered the modified cells without the agonist or ligand.

54. A modified cell comprising: a first nucleic acid that encodes modified signal regulatory protein-alpha (SIRPα) that interferes with an interaction between SIRPα and CD47, the modified SIRPα lacking a functional SIRPα intracellular domain; and
a second nucleic acid that encodes a chimeric antigen receptor (CAR) comprising an extracellular domain, a transmembrane domain, and an intracellular domain, and wherein the modified SIRPα and the CAR are expressed as gene products that are separate polypeptides.

55 A modified cell comprising: a CAR comprising an extracellular domain, a transmembrane domain, and an intracellular domain; and modified SIRPα that interferes with an interaction between SIRPα and CD47, the modified SIRPα lacking a functional SIRPα intracellular domain.

56. A method of modifying a cell, the method comprising: introducing a CAR into the cell, and wherein the comprises modified SIRPα that interferes with an interaction between SIRPα and CD47, the modified SIRPα lacking a functional SIRPα intracellular domain.

57. The modified cell or the method of any of embodiments 1-3, wherein the cell is a monocyte, macrophage, or dendritic cell.

58. The modified cell or the method of embodiment 4, wherein the cell is a macrophage.

59. The modified cell or the method of embodiment 5, wherein the intracellular domain of the modified SIRPα consisting essentially of the amino acid sequence of SEQ ID: 71 or 73.

60. The modified cell or the method of embodiment 5, wherein the modified SIRPα is over-expressed as compared to the wild-type receptor.

61. The modified cell or the method of embodiment 5, wherein the extracellular domain comprises an antigen binding domain binds an antigen comprising Epidermal growth factor receptor (EGFR), Variant III of the epidermal growth factor receptor (EGFRvIII), Human epidermal growth factor receptor 2 (HER2), Mesothelin (MSLN), Prostate-specific membrane antigen (PSMA), Carcinoembryonic antigen (CEA), Disialoganglioside 2 (GD2), Interleukin-13Ra2 (IL-13Ra2), Glypican-3 (GPC3), Carbonic anhydrase IX (CAIX), L1 cell adhesion molecule (L1-CAM), Cancer antigen 125 (CA125), Cluster of differentiation 133 (CD133), Fibroblast activation protein (FAP), Cancer/testis antigen 1B (CTAG1B), Mucin 1 (MUC1), Folate receptor-α (FR-α), CD19, FZD10, TSHR, PRLR, Muc 17, GUCY2C, CD207, CD3, CDS, B-Cell Maturation Antigen (BCMA), or CD4.

62. The modified cell or the method of embodiment 5, wherein the intracellular domain of the CAR comprises a co-stimulatory signaling region that comprises an intracellular domain of a co-stimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, and any combination thereof.

63. A pharmaceutical composition comprising the cell of embodiment 54 or 55and a pharmaceutically acceptable carrier.

64. Use of the modified cell of embodiment 54 or 55in the manufacture of a medicament for the treatment of a tumor or cancer in a subject in need thereof.

65. A method of treating a tumor in a subject, comprising administering to the subject a therapeutically effective amount of a pharmaceutical composition comprising the modified cell of embodiment 54 or 55.

66. A nucleic acid comprising a polynucleotide encoding one or more therapeutic agents that inhibit CD47 and/or CD24 signaling pathways.

67. A modified cell comprising a nucleic acid encoding one or more therapeutic agents that inhibit CD47 and/or CD24 signaling pathways.

68. A method for enhancing T cell response (e.g., expansion and/or activation) invitro and/or in vivo, the method comprising: introducing a nucleic acid encoding one or more therapeutic agents that inhibit CD47 and/or CD24 signaling pathway to obtain a modified cell; culturing the modified cell to obtain a population of modified cells; and contacting the population of modified cells with cells including an antigen, wherein the modified cells enhance the T cell response as compared to the cell contacted with modified cells not comprising the nucleic acid.

69. The nucleic acid, modified cell, or method of one of embodiments 66-68, wherein the one or more therapeutic agents comprise one or more recombinant proteins.

70. The nucleic acid, modified cell, or method of embodiment 69, wherein the one or more recombinant proteins comprise secretable/soluble proteins.

71. The nucleic acid, modified cell, or method of embodiment 69, wherein the one or more recombinant proteins comprise a dominant negative form of a corresponding protein.

72. The nucleic acid, modified cell, or method of any one of embodiment 69-71, wherein the one or more recombinant proteins comprise a recombinant protein of Siglec10 and/or a recombinant protein of SIRPα.

73. The nucleic acid, modified cell, or method of any one of embodiments 69-71, wherein the one or more therapeutic agents are or comprise a ligand for CD47 and/or a ligand for CD24.

74. The nucleic acid, modified cell, or method of one of embodiments 69-73, wherein the one or more recombinant proteins are associated with an oxygen-sensitive polypeptide domain.

75. The nucleic acid, modified cell, or method of embodiment 74, wherein the oxygen-sensitive polypeptide domain comprises a HIF VHL binding domain.

76. The nucleic acid, modified cell, or method of one of embodiments 66-75, wherein the nucleic acid encoding the one or more recombinant proteins that comprises a promoter comprising a binding site for a transcription modulator that modulates the expression and/or secretion of the therapeutic agent in the cell.

77. The nucleic acid, modified cell, or method of embodiment 76, wherein the transcription modulator is or includes Hif1a, NFAT, FOXP3, AP-1, Notch, and/or NFkB.

78. The nucleic acid, modified cell, or method of one of embodiments 66-77, wherein the nucleic acid encodes a binding molecule.

79. The modified cell or method of one of embodiments 66-77, wherein the modified cell comprises a binding molecule.

80. The nucleic acid, modified cell, or method of embodiment 79, wherein the nucleic acid is present in the modified cell in a recombinant DNA construct, in an mRNA, or in a viral vector.

81. The nucleic acid, modified cell, or method of embodiment 80, wherein the nucleic acid is an mRNA, which is not integrated into the genome of the modified cell.

82. The nucleic acid, modified cell, or method of one of embodiments 66-81, wherein the nucleic acid comprises a sequence encoding a cleavable peptide (e.g., 2A or IRES), which is disposed between at least two cytokines.

83. The nucleic acid, modified cell, or method of one of embodiments 66-81, wherein the one or more therapeutic agents are or comprise a secretable/soluble protein of Siglec10, a secretable/soluble protein of SIRPα, a dominant negative form of Siglec10, and/or a dominant negative form of SIRPα.

84. The nucleic acid, modified cell, or method of any one of embodiments 66-83, wherein the nucleic acid encodes a CAR, or the modified cell comprises a CAR.

85. The nucleic acid, modified cell, or method of embodiment 84, wherein the CAR comprises an extracellular domain, a transmembrane domain, and an intracellular domain, the extracellular domain binding an antigen.

86. The nucleic acid, modified cell, or method of embodiment 85, wherein the CAR binds tMUC 1, PRLR, CLCA1, MUC12, GUCY2C, GPR35, CR1L, MUC 17, TMPRSS11B, MUC21, TMPRSS11E, CD207, SLC30A8, CFC1, SLC12A3, SSTR1, GPR27, FZD10, TSHR, SIGLEC15, SLC6A3, KISS1R, QRFPR, GPR119, CLDN6, UPK2, ADAM12, SLC45A3, ACPP, MUC21, MUC16, MS4A12, ALPP, CEA, EphA2, FAP, GPC3, IL-13Ra2, Mesothelin, PSMA, ROR1, VEGFR-II, GD2, FR-α, ErbB2, EpCAM, EGFRvIII, PSCA, or EGFR.

87. The nucleic acid, modified cell, or method of embodiment 86, wherein the intracellular domain comprises a co-stimulatory domain that comprises an intracellular domain of a co-stimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, and one combination thereof.

88. The nucleic acid, modified cell, or method of embodiment 86, wherein the antigen is Epidermal growth factor receptor (EGFR), Variant III of the epidermal growth factor receptor (EGFRvIII), Human epidermal growth factor receptor 2 (HER2), Mesothelin (MSLN), Prostate-specific membrane antigen (PSMA), Carcinoembryonic antigen (CEA), Disialoganglioside 2 (GD2), Interleukin-13Ra2 (IL-13Ra2), Glypican-3 (GPC3), Carbonic anhydrase IX (CAIX), L1 cell adhesion molecule (L1-CAM), Cancer antigen 125 (CA125), Cluster of differentiation 133 (CD133), Fibroblast activation protein (FAP), Cancer/testis antigen 1B (CTAG1B), Mucin 1 (MUC1), Folate receptor-a (FR-a), CD19, FZD10, TSHR, PRLR, Muc 17, GUCY2C, CD207, CD3, CDS, B-Cell Maturation Antigen (BCMA), or CD4.

89. A composition for treating a subject having cancer, the composition comprising a first population of cells and a second population of cells, wherein the first population of cells comprising a nucleic acid encoding a binding molecule binding a solid tumor antigen, a second population of cells comprising the nucleic acid of one of embodiments 66-88.

90. A composition for treating a subject having cancer, the composition comprising a first population of cells and a second population of cells, wherein the first population of cells comprising a nucleic acid encoding a binding molecule binding a white blood antigen, a second population of cells expressing and/or secreting the one or more therapeutic agents of any one of embodiments 66-89.

91. A method for treating an autoimmune disease in a human patient, the method comprising: administrating to the human patient a pharmaceutically effective amount of a population of T cells of the human patient that express a chimeric antigen receptor (CAR) that comprising the amino acid sequence of any of SEQ ID NO: 117-125.

92. A method for treating an autoimmune disease in a human patient, the method comprising: administrating to the human patient a pharmaceutically effective amount of a population of T cells of the human patient that express a chimeric antigen receptor (CAR) that binds an antigen associated with B cells.

93. The method of embodiment 92, wherein the autoimmune disease is an autoimmune disease associated with B cell proliferation and immunoglobulin secretion.

94. The method of embodiment 92, wherein the autoimmune disease is Systemic Lupus Erythematosus (SLE), Rheumatoid Arthritis (RA), Type 1 Diabetes (T1D), Sjögren's syndrome, or Multiple Sclerosis (MS).

95. The method of embodiment 92, wherein the antigen is BAFF, APRIL, CD19, CD20, CD22, a B cell receptor (BCR), B cell maturation antigen (BCMA) or CD79.

96. The method of embodiment 92, wherein the antigen is CD19, CD20 or BAFF.

97. The method of embodiment 92, wherein the pharmaceutically effective amount of a population of T cells is about 10⁴ to 10⁹ cells per kg body weight of the human patient.

98. The method of embodiment 92, wherein the pharmaceutically effective amount of a population of T cells is about 10⁵ to 10⁶ cells per kg body weight of the human patient.

99. The method of embodiment 92, wherein the pharmaceutically effective amount of a population of T cells is about 10⁶ to 10⁷ cells per kg body weight of the human patient.

100. The method of embodiment 92, wherein the pharmaceutically effective amount of a population of T cells is about 10⁴ to 10⁵ cells per kg body weight of the human patient.

101. The method of embodiment 92, wherein the pharmaceutically effective amount of a population of T cells is about 10⁷ to 10⁸ cells per kg body weight of the human patient.

102. The method of embodiment 92, wherein the pharmaceutically effective amount of a population of T cells is about 10⁸ to 10⁹ cells per kg body weight of the human patient.

103. The method of embodiment 92, wherein the CAR comprises the amino acid sequence of SEQ ID NO: 1-9.

104. Use of a population of T cells expressing a CAR that binds an antigen associated with B cells for use in the treatment of autoimmune disease.

105. The use of embodiment 104, the autoimmune disease is Systemic Lupus Erythematosus (SLE), Rheumatoid Arthritis (RA), Type 1 Diabetes (T1D), Sjögren's syndrome, or Multiple Sclerosis (MS).

106. The use of embodiment 104 or 105, wherein the autoimmune disease is SLE.

107. The use of any one of embodiments 104-106, wherein the T cells include the amino acid sequence of any one of SEQ ID NO: 1-9.

108. The use of any one of embodiments 104-107, wherein the antigen is BAFF, APRIL, CD19, CD20, CD22, B cell receptor (BCR), B cell maturation antigen (BCMA) or CD79.

109. The use of any one of embodiments 104-108, wherein the antigen is CD19, CD20 or BCMA.

110. The use of any one of embodiments 104-109, wherein the pharmaceutically effective amount of the population of T cells is about 10⁴ to 10⁹ cells per kg body weight of the human patient.

111. The use of any one of embodiments 104-110, wherein the pharmaceutically effective amount of the population of T cells is about 10⁵ to 10⁶ cells per kg body weight of the human patient.

112. The use of any one of embodiments 104-111, wherein the pharmaceutically effective amount of the population of T cells is about 10⁶ to 10⁷ cells per kg body weight of the human patient.

113. The use of any one of embodiments 104-112, wherein the pharmaceutically effective amount of the population of T cells is about 10⁴ to 10⁵ cells per kg body weight of the human patient.

114. The use of any one of embodiments 104-113, wherein the pharmaceutically effective amount of the population of T cells is about 10⁷ to 10⁸ cells per kg body weight of the human patient.

115. The use of any one of embodiments 104-114, wherein the pharmaceutically effective amount of the population of T cells is about 10⁸ to 10⁹ cells per kg body weight of the human patient.

116. The use of any one of embodiments 104-115, wherein the CAR comprises the amino acid sequence of SEQ ID NO: 117-125.

117. The use of any one of embodiments 104-116, wherein the use further includes measuring a level of: a urine protein, a serum anti-double-stranded DNA antibody, renal impairment, and/or damage to skin of the subject.

118. The use of any one of embodiments 104-117, wherein the level of the urine protein, the serum anti-double-stranded DNA antibody, the renal impairment, and/or the damage to the skin of the subject decreases.

119. A population of modified cells effective for expanding and/or maintaining the modified cells in a patient, wherein the population of modified cells comprise at least two different modified cells: a first modified cell comprising a first antigen binding domain for expanding, maintaining the modified cells, and/or killing B cells; and a second modified cell comprising a second antigen binding domain for killing a target cell, such as a T cells causing an autoimmune disease, preferably, wherein the modified cells are modified T cells, preferably, wherein, the at least two different modified cells include two different modified T cells, two different modified immune cells, or a combination thereof, and/or preferably, wherein the modified immune cells include modified T cells, DC cells, and/or macrophages.

120. The population of modified cells of embodiment 119, wherein the antigen binding domains bind different antigens.

121. The population of modified cells of embodiment 119, wherein the population of modified cells further comprises a third modified cell expressing at least two different antigen binding domains, a first antigen binding domain for expanding and/or maintaining the modified cells and a second antigen binding domain for killing a target cell, and wherein the two different antigen binding domains are expressed on the same cell.

122. The population of modified cells of embodiment 119, wherein the population of modified cells comprises a modified cell expressing an antigen binding domain for killing a target cell and a modified cell expressing at least two antigen binding domains, a first antigen binding domain for expanding and/or maintaining the modified cells and a second antigen binding domain for killing a target cell, and wherein the two different antigen binding domains are expressed on the same modified cell.

123. The population of modified cells of embodiment 119, wherein the population of modified cells includes a modified cell expressing an antigen binding domain for expanding and/or maintaining the modified cells and a modified cell expressing at least two antigen binding domains, a first antigen binding domain for expanding and/or maintaining the modified cells and a second antigen binding domain for killing a target cell, and wherein the two different antigen binding domains are expressed on the same modified cell.

124. The population of modified cells of any one of embodiments 119-123, wherein the modified cell is a modified T cell, a modified NK cell, a modified macrophage, or a modified dendritic cell.

125. The population of modified cells of any one of embodiments 119-124, wherein the antigen binding domain for expanding/or and maintaining the modified cells bind the surface antigen of a WBC, and the antigen binding domain for killing a target cell binds a tumor antigen.

126. The population of modified cells of embodiment 125, wherein the WBC is a B cell.

127. The population of modified cells of embodiment 125, wherein the cell surface antigen of the WBC is CD19, CD22, CD20, BCMA, CD5, CD7, CD2, CD16, CD56, CD30, CD14, CD68, CD11b, CD18, CD169, CD1c, CD33, CD38, CD138, or CD13.

128. The population of modified cells of any one of embodiments 119-127, wherein the second antigen is T cell one or more domains associated with HLA-peptide complex (HPC).

129. The population of modified cells of embodiment 125, wherein the cell surface antigen of the WBC is CD19, CD20, CD22, or BCMA or wherein the WBC is a B cell and the cell surface antigen of the B cell is CD19.

130. A composition comprising a first population of cells comprising a first CAR binding a first antigen and a second population of cells comprising a second CAR binding a second antigen, wherein the second antigen corresponds to an ADHPC, e.g., SEQ ID NO: 10 and 11, and the first antigen and second antigen are different antigens.

131. The composition of embodiment 130, wherein the first population of cells does not comprise the second CAR, and/or the second population of cells does not comprise the first CAR.

132. The composition of embodiment 131, wherein the composition further comprises a third population of cells comprising the first CAR and the second CAR.

133. The composition of embodiment 130, wherein the second population of cells further comprises the first CAR, and the first population of cells do not comprise the second CAR; or the first population of cells further comprises the second CAR.

134. The composition of embodiment 130, wherein second population of cells does not comprise the first CAR, and the first population of cells comprise the second CAR.

135. A method of enhancing expansion of the second population of cells, wherein the second population of cells are cells comprising an ADHPC, the method comprising administering an effective amount of the composition of any one of embodiments 130-134 to a subject having a form of cancer associated with the ADHPC.

136. A method of enhancing T cell response in a subject or treating a subject having cancer, the method comprising administering an effective amount of the composition of any one of embodiments 130-134 to the subject having a form of autoimmune disease associated with the ADHPC.

137. A method of enhancing expansion of cells in a subject, the method comprising: contacting cells with a first vector comprising a first nucleic acid sequence encoding a first CAR and a second vector comprising a second nucleic acid sequence encoding a second CAR to obtain the composition of any one of embodiments 130-134; and administering an effective amount of the composition to the subject having a form of autoimmune diseases associated with the ADHPC.

138. A method of enhancing T cell response in a subject in need thereof or treating a subject having an autoimmune disease, the method comprising: contacting cells with a first vector comprising a first nucleic acid sequence encoding a first CAR and a second vector comprising a second nucleic acid sequence encoding a second CAR to obtain the composition of any one of embodiments 130-134; and administering an effective amount of the composition to the subject having a form of autoimmune diseases associated with the ADHPC.

139. A method of enhancing expansion of cells in a subject, the method comprising: administering an effective amount of the first population of cells of the composition of any one of embodiments 130-134; and administering an effective amount of the second population of cells.

140. The method of any one of embodiments 137-139, wherein the first vector and the second vector comprise lentiviral vectors.

141. The composition or the method of any one of embodiments 130-140, wherein the first antigen is or comprises a surface molecule of a white blood cell, such as WBC.

142. The composition or the method of any one of embodiments 130-141, wherein the cells are modified T cells, modified NK cells, modified macrophages, or modified dendritic cells.

143. The composition or the method of embodiment 141, wherein the WBC is a granulocyte, a monocyte, or a lymphocyte.

144. The composition or the method of embodiment 143, wherein the WBC is a B cell.

145. The composition or the method of embodiment 144, wherein the cell surface antigen of the WBC is CD19, CD22, CD20, BCMA, CD5, CD7, CD2, CD16, CD79A/B, CD56, CD30, CD14, CD68, CD11 b, CD18, CD169, CD1c, CD33, CD38, CD138, or CD13.

146. The composition or the method of embodiment 143, wherein the cell surface antigen of the WBC is CD19, CD20, CD22, or BCMA.

147. The composition or the method of embodiment 143, wherein the cell surface antigen of the WBC is CD19.

148. The composition or the method of embodiment 143, wherein the first antigen corresponds to ADHPC.

149. The composition or the method of embodiment 148, wherein the ADHPC is a native HLA-peptide or artificial HLA-peptide.

150. A fusion protein, e.g., polyspecific binding molecule, e.g., polyspecific antibody, comprising:
a first antigen binding domain targeting a receptor of an antigen of T cells; and
a second antigen binding domain targeting a receptor of an ADHPC.

151. The fusion protein of embodiment 150, wherein the receptor comprises CD3, CD28, 41BB, and/or OX40.

152. A composition comprising the fusion protein of embodiment 150 or 151.

153. A polynucleotide encoding the fusion protein of any preceding suitable embodiments.

154. A host cell comprising the fusion protein of any preceding suitable embodiments.

155. A modified cell comprising the fusion protein of any preceding embodiments.

156. A composition comprising the modified cells of embodiment 155.

157. A method for treating or causing or inducing a T cell response in a subject having autoimmune disease, the method comprising administering an effective amount of the composition comprising modified cells of embodiment 156.

158. A method for treating cancer, causing or inducing a T cell response in a subject having the cancer, or enhancing the T cell response, the method comprising administering an effective amount of the composition of embodiment 152.

159. A method for treating or causing or inducing a T cell response in a subject having an autoimmune disease, the method comprising administering an effective amount of the composition comprising fusion proteins of any preceding suitable embodiments.

160. A method for treating cancer, causing or inducing a T cell response in a subject having the autoimmune disease, or enhancing the T cell response, the method comprising administering an effective amount of the composition comprising fusion proteins of any preceding suitable embodiments.

161. The method of any suitable preceding embodiments, wherein the polyspecific binding molecule, e.g., polyspecific antibody, comprise a bispecific binding molecule (such as BITE) comprising a binding domain binding a WBC, white blood antigen, e.g., CD19, antigen and a binding domain binding CD3.

162. The method or composition of any suitable preceding embodiments, wherein the polyspecific binding molecule or the CAR comprises at least one of the sequence listed in the Table 6.

163. A method of removing T cells from the body of a subject, the method comprising administering to the subject with an effective amount of the composition comprising modified cells or the fusion protein of any suitable preceding embodiments.

164. A composition comprising an antigen that a CAR binds and a CD40 activator, wherein the CAR is in a modified T cell.

165. The composition of embodiment 164, wherein the CD40 activator is a CD40 ligand or CD40 agonist.

166. The composition embodiment 164 or 165 for use in enhancing a T cell response of modified T cells comprising a nucleic acid encoding a CAR and a nucleic acid encoding an extracellular domain of CD40.

167. The composition of any one of embodiments 164-167 for use in a method of enhancing a T cell response, the method comprising:
introducing a nucleic acid encoding a chimeric antigen receptor (CAR) and a polynucleotide encoding an extracellular domain of CD40 into T cells to obtain modified T cells; and
contacting the modified T cells with the composition of any one of embodiments 164-166, thereby inducing a T cell response, wherein the T cell response is greater than the T cell response induced by contacting the modified T cells with a composition comprising the antigen without the CD40 activator.

168. The composition of embodiment 166 or 167, wherein the nucleic acid encoding the extracellular domain of CD40 comprises nucleic acid sequence SEQ ID NO: 36. 167.

169. The composition of any one of embodiments 166-168, wherein the T cell response comprises T cell expansion and/or T cell activation.

170. The composition of any one of embodiments 164-169, wherein the CAR comprises an antigen binding domain, a transmembrane domain, and an intracellular signaling domain.

171. The composition of any one of embodiments 164-169, wherein the antigen binding domain binds a tumor antigen, the tumor antigen comprising TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LACE-1a, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, or IGLL1.

172. The composition of any one of embodiments 164-171, wherein the CAR comprises an intracellular signaling domain comprising a co-stimulatory signaling domain, or a primary signaling domain and a co-stimulatory signaling domain, and wherein the co-stimulatory signaling domain comprises a functional signaling domain of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, or NKG2D.

173. The composition of any one of embodiments 164-172, wherein the CAR comprises an intracellular domain comprising a CD3 zeta signaling domain.

174. The composition of any one of embodiments 166-173, wherein the nucleic acid is an mRNA, which is not integrated into the genome of the modified cell.

175. The composition of any one of embodiments 166-174, wherein the nucleic acid encoding CD40 is associated with an oxygen-sensitive polypeptide domain, and optionally wherein the oxygen-sensitive polypeptide domain comprises Hif1a VHL binding domain.

176. The composition of any one of embodiments 166-175, wherein the nucleic acid is regulated by a promoter comprising a binding site for a transcription modulator that modulates the expression and/or secretion of the therapeutic agent in the cell, and optionally wherein the transcription modulator comprises one or more of Hif1a, NFAT, FOXP3, and NFkB.

177. The composition of any one of embodiments 164-176, wherein the modified T cells comprise a nucleic acid encoding a dominant negative form of an inhibitory immune checkpoint molecule or a receptor thereof, and optionally wherein the inhibitory immune checkpoint molecule comprises programmed death 1 (PD-1), cytotoxic T lymphocyte antigen-4 (CTLA- 4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIRD, natural killer cell receptor 2B4 (2B4), or CD160.

178. The composition of any one of embodiments 164-177, wherein the modified T cells have a reduced expression of endogenous TRAC gene.

179. The composition of any one of embodiments 164-178, wherein the modified T cells comprise a nucleic acid encoding hTERT or a nucleic acid encoding SV40LT, or a combination thereof.

180. The composition of any one of embodiments 164-179, wherein the modified T cells comprise an exogenous nucleic acid encoding a therapeutic agent, and the therapeutic agent comprises one or more of IL-1P, IL-2, IL-4, IL-5, IL-6, IL-7, IL-8, IL-10, IL-12, IL-13, IL-15, IL-17, IL-1Ra, IL-2R, IFNγ, MIP-In, MIP-IP, MCP-1, TNFα, GM-CSF, GCSF, CXCL9, CXCL10, CXCR factors, VEGF, RANTES, EOTAXIN, EGF, HGF, FGF-P, and ferritin.

181. The composition of any one of embodiments 164-180, wherein the modified T cells comprise at least one of 1) modified T cells comprising a first CAR binding a surface molecule of a white blood cell and a second CAR binding a solid tumor antigen, 2) modified T cells comprising the first CAR without the second CAR, and 3) modified T cell comprising the second CAR without the first CAR.

182. The composition of any one of embodiments 164-181, wherein the T cells are derived from a subject having cancer or a healthy donor.

### EXAMPLES

Lentiviral vectors that encode individual CAR molecules were generated and transfected with T cells, and described below. Techniques related to cell cultures, construction of cytotoxic T lymphocyte assay can be found in “Control of large, established tumor xenografts with genetically retargeted human T cells containing CD28 and CD137 domains,” PNAS, Mar. 3, 2009, vol. 106 no. 9, 3360-3365 and “Chimeric Receptors Containing CD137 Signal Transduction Domains Mediate Enhanced Survival of T Cells and Increased Antileukemic Efficacy In Vivo,” Molecular Therapy, August 2009, vol. 17 no. 8, 1453-1464, which are incorporated herein by reference in its entirety.

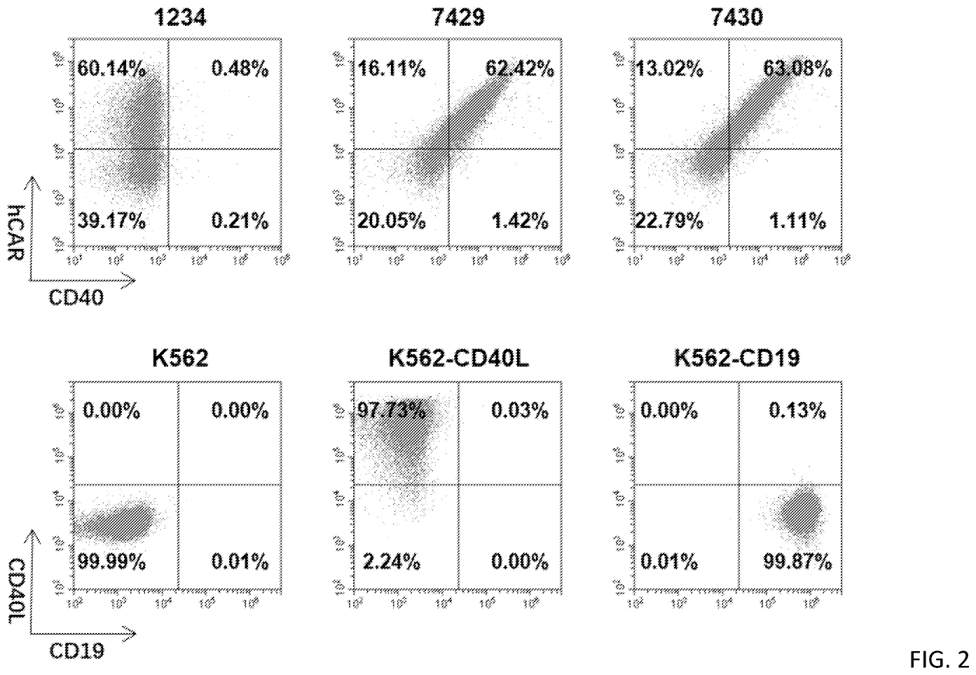

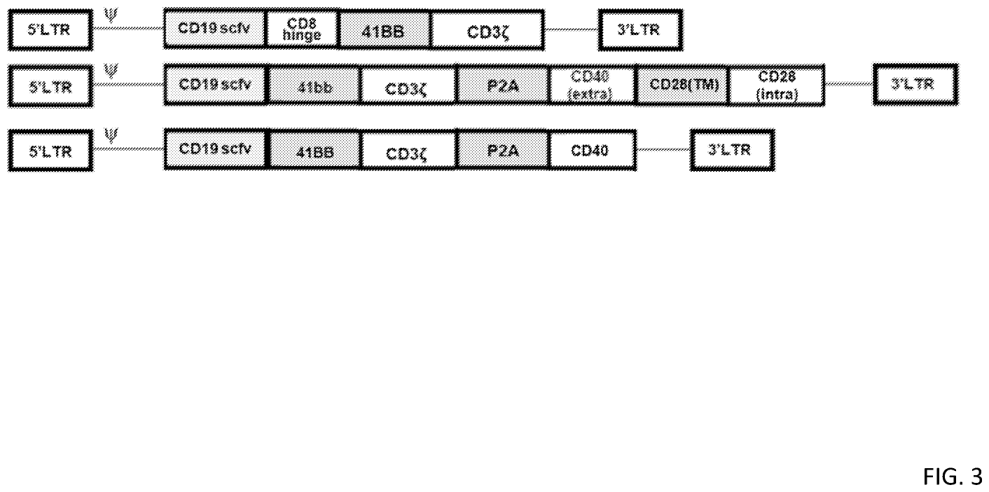

FIG. 2 shows CAR expression ratio and cell phenotypes that were determined using flow cytometry. FIG. 3 shows a schematic diagram of various vectors transduced into T cells. Information regarding vectors and cells are provided in Table 2, and experimental designs are provided in Table 3.

TABLE 2

| Cell IDs | Construct |
|---|---|
| 1234 | h19CAR(humanized CD19 CAR) |
| 7429 | H19bbz(humanized CD19 + 4-1BB)-2a-CD40(extracellular)- |
| | CD28(TM)-CD28(intracellular) |
| 7430 | H19bbz-2a-CD40(full-length) |

On Day 0, the peripheral blood of healthy volunteers was taken, and CD3+T cells were sorted with the Pan T Cell Isolation Kit. The isolated CD3+T cells were then mixed with 100 ul TtransAct® per 1×10⁷ T cells. On Day 1, the activated CD3+T cells were transduced with lentivirus vectors encoding h19 CAR (MOI=10.00), lentivirus vectors encoding H19bbz-2a-CD40(extracellular)-CD28(TM)-CD28(intracellular) CAR (MOI=60.17), and lentivirus vectors encoding H19bbz-2a-CD40 CAR(MOI=60.35). Cells of the control group were not transfected with vectors. Since scFv of CD19 CAR is derived from a humanized antibody, they were detected with human CAR antibodies. On day 6, flow cytometry was performed, and results are shown in FIG. 2.

TABLE 3

| | Substrate cell | | | | |
|---|---|---|---|---|---|
| T CELL | K562 | K562-CD40L | K562-CD19 | E:T | System |
| NT | + | − | − | 3:1 | 24-well |
| NT | − | + | − | | plate 800 |
| NT | − | − | + | | ul x-vivo |
| 1234 | + | − | − | | without |
| 1234 | − | + | − | | IL-2 |
| 1234 | − | − | + | | added |
| 7429 | + | − | − | | |
| 7429 | − | + | − | | |
| 7429 | − | − | + | | |
| 7430 | + | − | − | | |
| 7430 | − | + | − | | |
| 7430 | − | − | + | | |

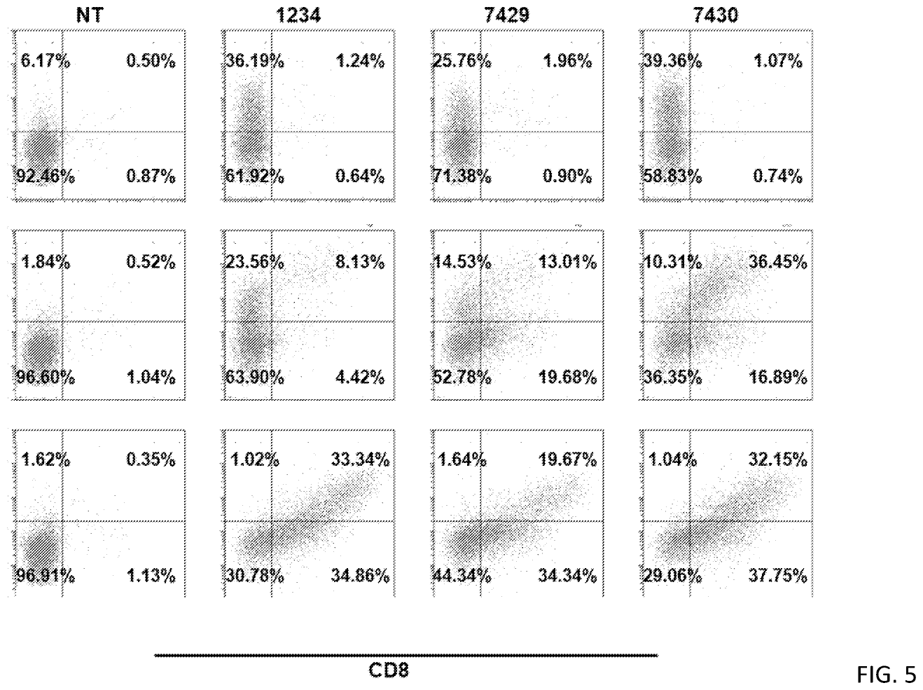

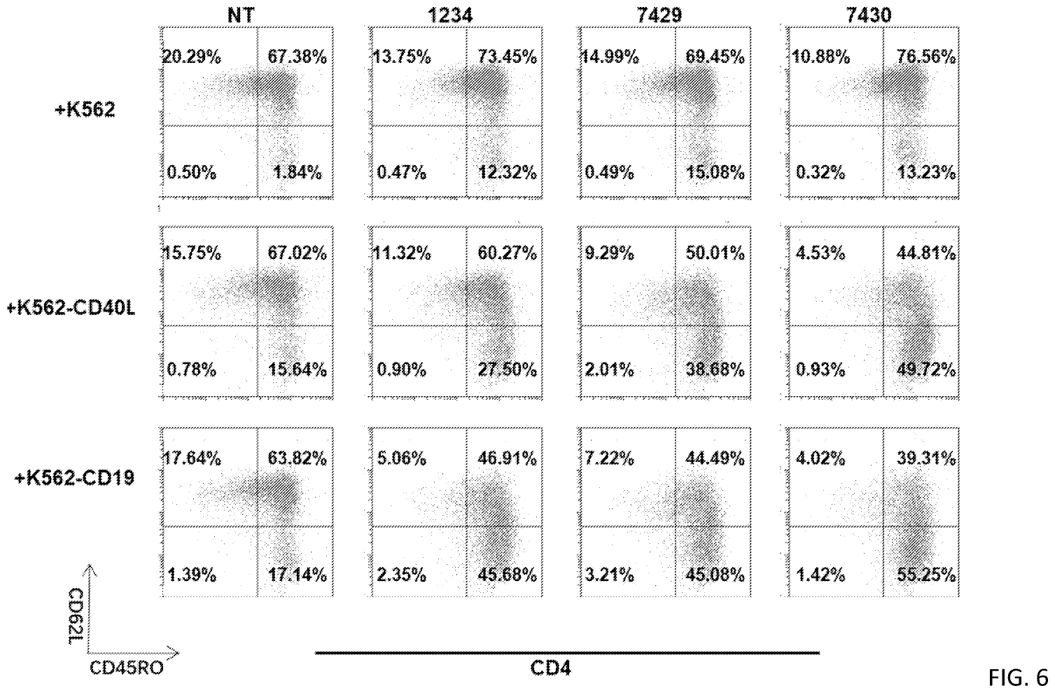

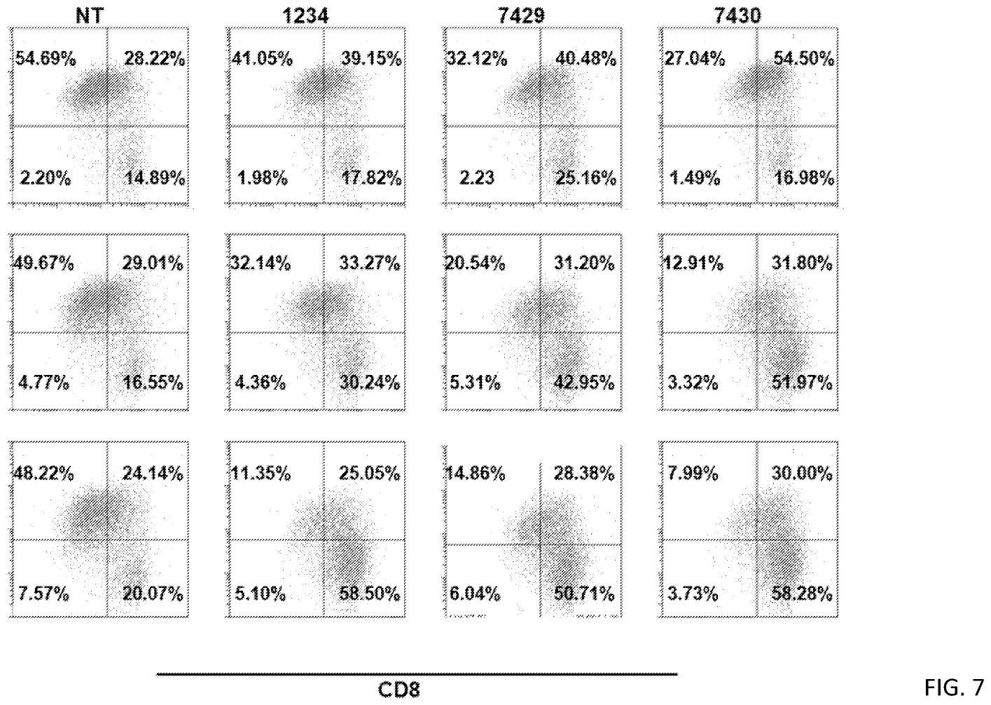

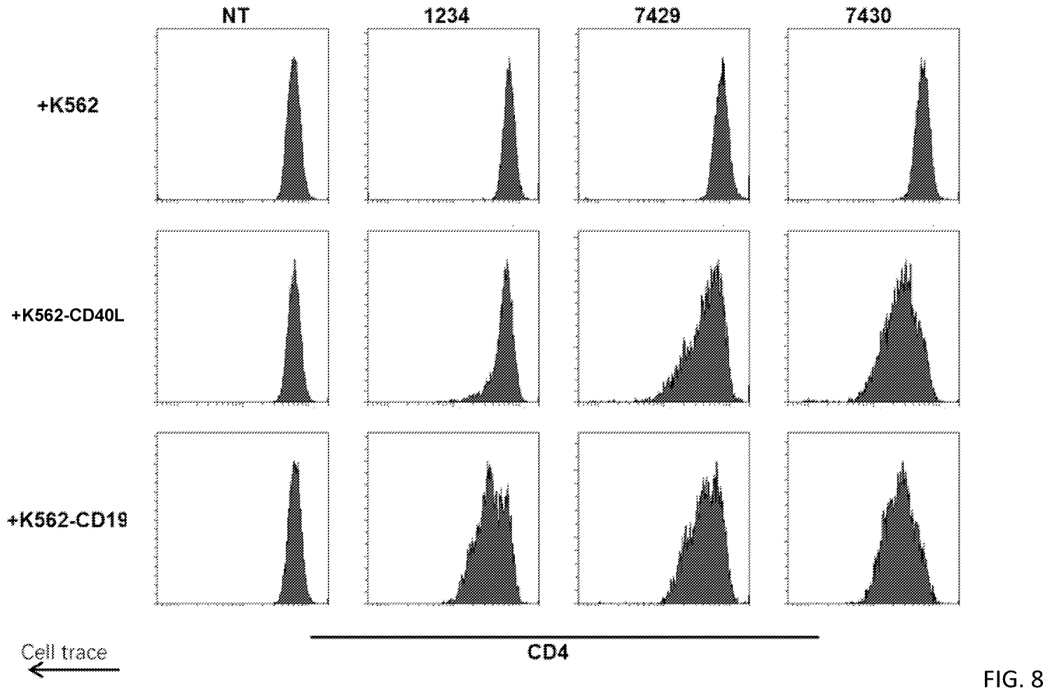

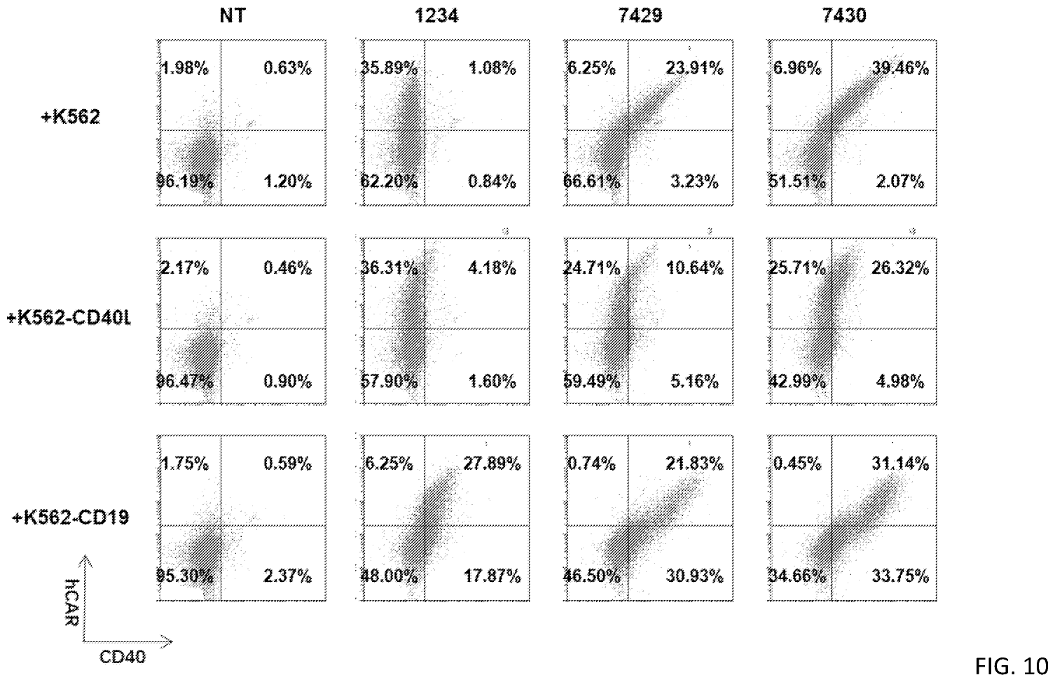

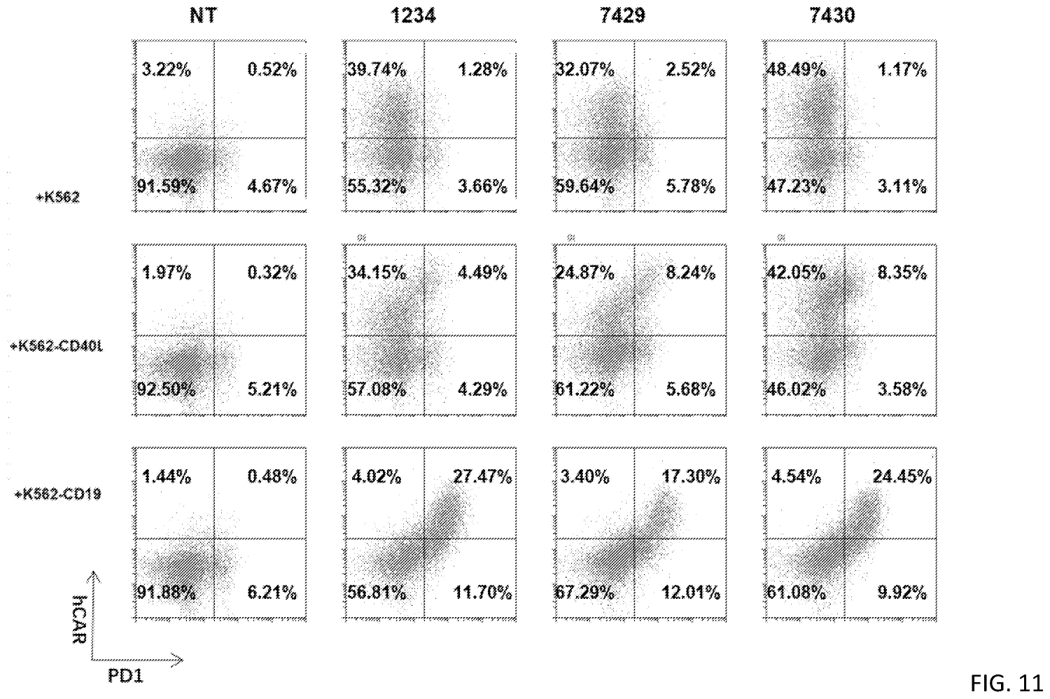

FIGS. 4 and 5 show results of flow cytometry analysis of the expression of CD137, indicating that K562-CD40L can significantly activate 7429 and 7430. Compared with 1234, 7429 and 7430 showed significant activation. FIGS. 6 and 7 show results of flow cytometry analysis of the overexpression of CD40 on T cells that were stimulated by K562-CD40L to differentiate from memory cells to effector cells. Compared with 1234, 7429 and 7430 showed more cells in the differentiated status. FIGS. 8 and 9 show the overexpression of CD40 on T cells that proliferated significantly after being stimulated by K562-CD40L. CellTrace™ was used to measure amplification levels. As compared to 1234, 7429 and 7430 showed higher amplification levels. FIGS. 10 and 11 show the results of flow cytometry analysis of T cells including vectors encoding CD40 that down-regulated CD40 expression and PD-1 expression after receiving CD40L stimulation as compared to the T cells without CD40L stimulation. The downregulation improves the safety of cellular therapy. Compared with 1234, neither 7429 nor 7430 showed significant up-regulation of PD1 expression. Also, co-cultivation with K562 expressing CD40L showed that the CD40 expression on CAR T cells was down-regulated, further ensuring CAR T therapy's safety for clinical uses.

These results demonstrated that CD40L can enhance the activation and expansion of T cells expressing CAR and CD40 extracellular domain. Thus, CD40 agonists can replace CD40L to enhance the activation and expansion. Various CD40 agonists are commercially available. CD40 agonists have a certain half-life, making them controllable and safe.

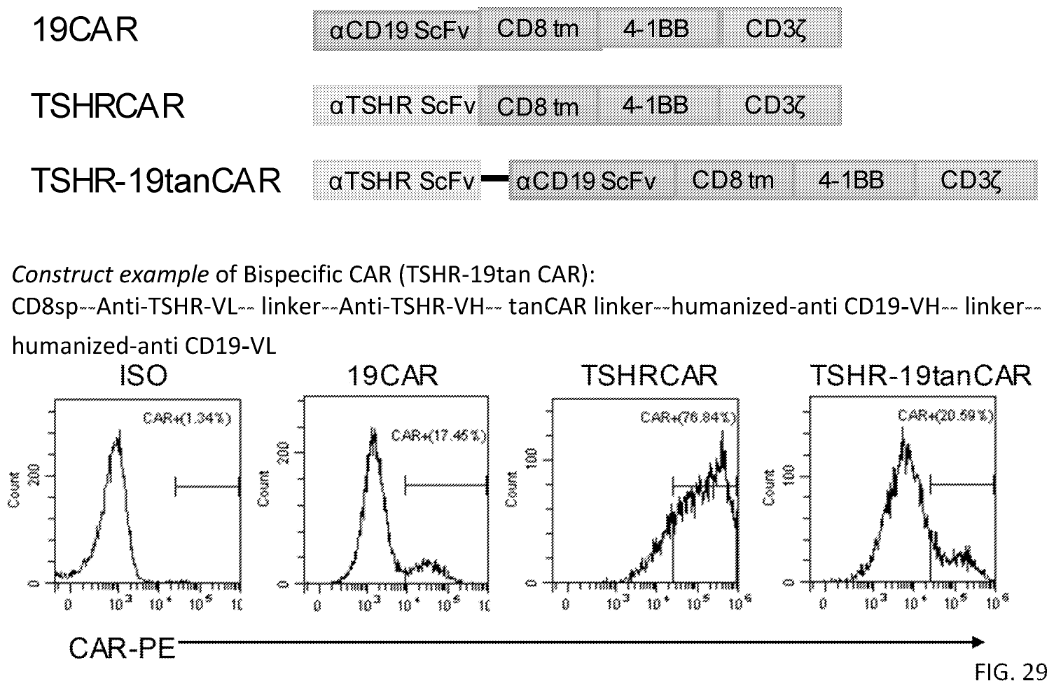

On Day 0, peripheral blood was drawn from healthy volunteers and sorted to collect CD3+ T cells. CD3/CD28 Dynabeads were added to the collected CD3+ T cells in a 1:1 ratio. On Day 1, the activated CD3+ T cells were transfected with various vectors. Structure and sequence information for the constructs are provided in FIG. 29. On Day 2, culture media were changed; the lentivirus was removed; and the cells were resuspended in fresh media. On Day 5, flow cytometry analysis on CAR expression was performed.

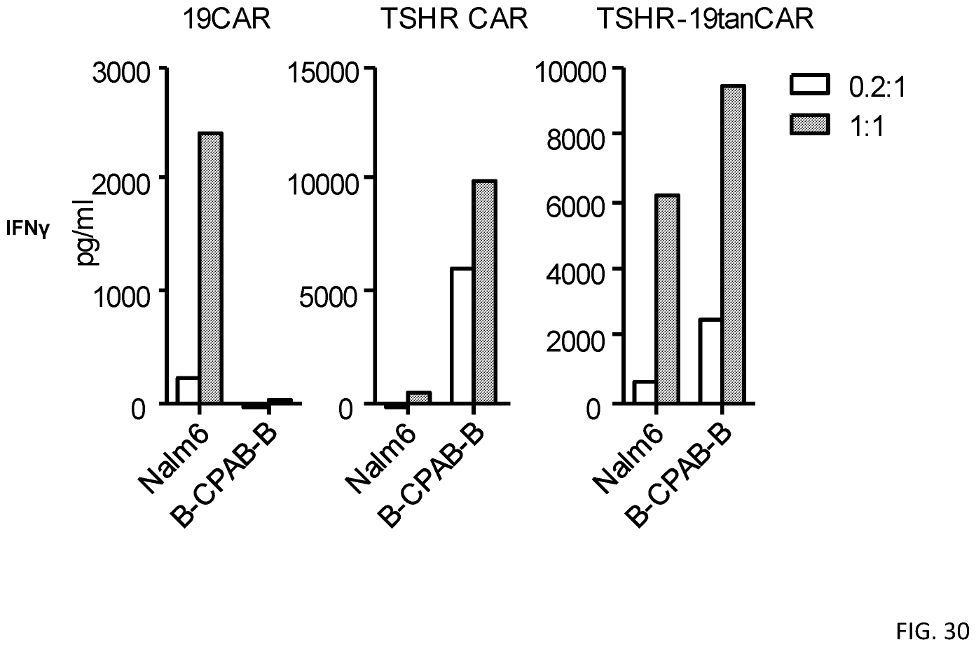

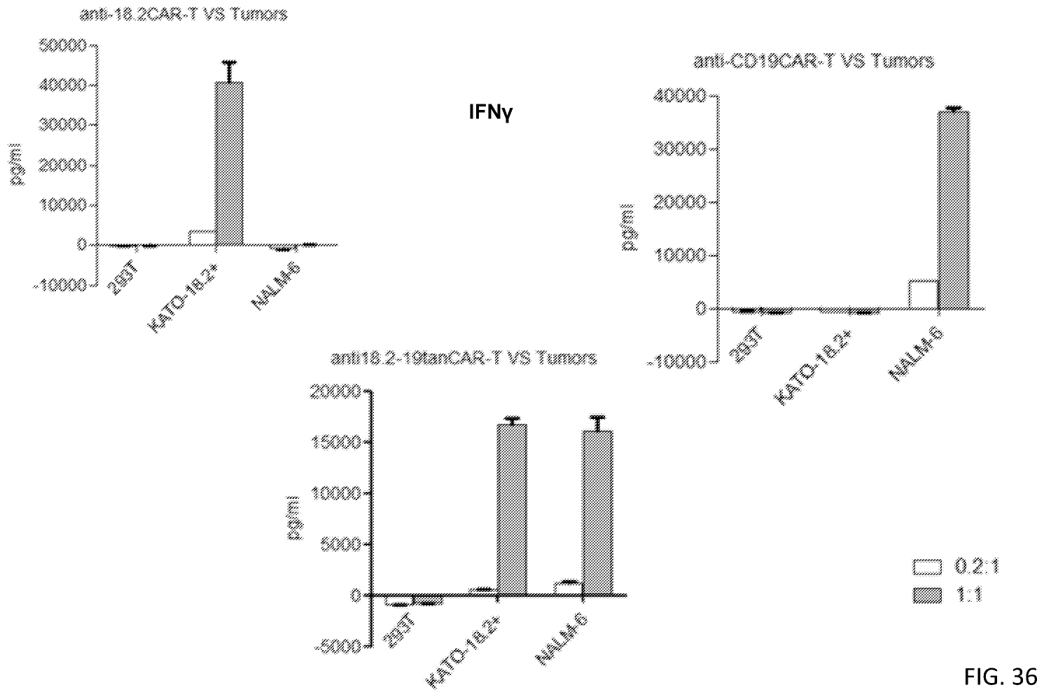

CAR T cells and Nalm6 or B-CPAB-B tumor cells were co-cultured for 24 hours (hrs), and the supernatant was collected. IFNγ release was detected. Nalm6 was a CD19-positive tumor cell, and B-CPAB-B was a TSHR positive tumor cell. As shown in the left panel of FIG. 30, CD19 CART cells released more IFNγ in response to Nalm6 as compared to that released in response to B-CPAB-B. As shown in the middle panel, TSHR CAR T cells released more IFNγ in response to B-CPAB-B as compared to that released in response to Nalm6. As shown in the right panel, bispecific CAR T cells released a significant amount of IFNγ in response to Nalm6 and B-CPAB-B, separately. These results indicated that bispecific CAR T cells can be stimulated by both CD19-positive or TSHR-positive cells. Similar cytokine release assays were performed and showed that bispecific CAR T (CLDN18.2-CD19 bispecific CAR or CLDN18.2-19tan CAR) cells were activated by both Nalm6 and cells expressing CLDN18.2 (FIG. 36).

On Day 0, peripheral blood was drawn from healthy volunteers. CD3+T cells were sorted with Pan T Cell Isolation kit and activated by CD3/CD28 Dynabeads at a ratio of 3:1. On Day 1, the activated CD3+ T cells were infected. Several groups of cells were infected with vectors shown in Table 5, and the remaining cells were used as NT (non-transfected). On Day 2, the lentivirus and the Dynabeads were removed, and the culture media were replaced. On Day 6, the CAR ratio and cell phenotype of CAR T cells were measured in each group using flow cytometry assay. Since anti-ACPP antibodies are humanized antibodies, and anti-MUC1 antibodies are murine antibodies, rabbit anti-human CAR antibodies and rabbit anti-mouse CAR antibodies were used to detect expression of these two scFvs, respectively. On Day 7, the experiment was carried out according to Table 5. The samples were flow-stained after 24 hours of full activation. The supernatant was collected for detection of Cytometric Bead Array (CBA), and carboxyfluorescein succinimidyl ester (CFSE) staining was performed to observe the proliferation. The cells were co-cultured with fluorescent substrate cells, and the survival of cells with fluorescent substrates was observed to determine the killing effect.

TABLE 4
CAR T cells and substrate cells

| Cells (ID) | Vectors |
|---|---|
| 6917 | CAR-5e5-28z |
| 6921 | CAR-ACPP-28z |
| 2529 | TanCAR 28z-ACPP + G4S + 5e5LH |
| 2530 | TanCAR 28z -ACPP + G4S + 5e5HL |
| 2533 | TanCAR 28z 5e5 + G4S + ACPP LH |
| 2534 | TanCAR 28z 5e5 + G4S + ACPP HL |
| MCF-7 | Breast cancer cell lines with high expression of tnMUC1 |
| PC3-ACPP | Human prostate cancer cells that overexpress ACPP |
| 2407 | tMUC1CAR-4-1BB |
| 163 | CLDN 18.2 CAR |
| 2517 | TanCAR 5E5 LH + G4S + 163LH |
| 1604 | TSHR - 4-1BB |
| 2407 | t MUC1 - 4-1BB |
| 2519 | TSHR - G4S - tMUC1 - 4-1BB |
| 2521 | tMUC1 - G4S - TSHR - 4-1BB |
| Notes | 5e5: GUCY2C antibody; 28z: CD28 and CD3zeta; 163: |
| | CLDN18.2 antibody; G4S: linker |

TABLE 5
cells for co-culturing assay

| | | | hCAR+ | mCAR+ | |
|---|---|---|---|---|---|
| ID | Vectors | MOI | (ACPP) | (tMUC1) | Co-culture |
| 6917 | CAR-5e528z | 13.15 | | 62.2 | Co-cultured |
| 6921 | CAR-ACPP-28z | 23.05 | 61.18 | | with |
| 2529 | TanCAR 28z | 51.65 | 55.21 | 49.54 | substrate |
| | ACPP + G4S + 5e5LH | | | | cells (MCF- |
| 2530 | TanCAR 28z | 52.49 | 70.51 | 60.49 | 7, PC3- |
| | ACPP + G4S + 5e5HL | | | | ACPP, |
| 2533 | TanCAR 28z 5e5 + | 51.04 | 55.94 | 63.1 | 293T) |
| | G4S + ACPP LH | | | | |
| 2534 | TanCAR 28z 5e5 + | 52.28 | 34.21 | 31.47 | |
| | G4S + ACPP HL | | | | |
| 2407 | tMUC1CAR-41bb | 8.44 | | 25.68 | Co-cultured |
| 163 | CLDN 18.2CAR | 64.8 | | 41.06 | with |
| 2517 | TanCAR 5E5 | 59.7 | | 44.42 | substrate |
| | LH + G4S + 163LH | | | | cells (MCF- |
| | | | | | 7, KATO3+, |
| | | | | | 293T) |

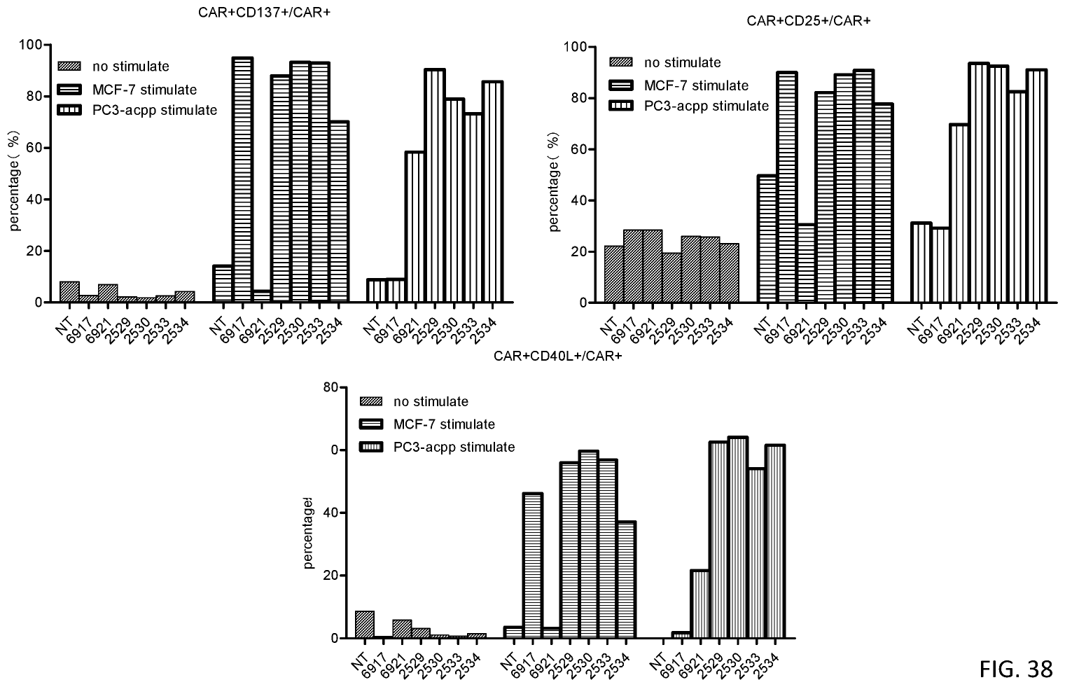

The results of the flow cytometry assay showing expression of several markers (i.e., CD137, CD25, and CD40L) on CART cells and tanCAR T cells are shown in FIG. 38. NT, 6917, 6921, 2529, 2530, 2533, 2534, and substrate cells (MCF-7, PC3-Acpp, 293T cells) were co-cultured for 24 hours. Flow cytometry assay was performed on Day 8. CAR T cells and three substrate cells (293T, MCF-7, PC3-Acpp) were co-cultured for 24 hrs. Flow cytometry assay was performed after the activation of CAR T cells. As shown in FIG. 38, the vertical coordinates are CAR+CD137+ cells (the total CAR+ cells) and CAR+CD25+ (total CAR+ cells), respectively. From the expression of CD137 and CD25, four types of tanCAR cells were effectively activated by corresponding substrate cells. The statistical analysis of the expression of CD0L by flow cytometry was performed after CAR T cells were co-cultured with substrate cells (293T, MCF-7 and PC3-Acpp) for 24hrs. Four types of tanCAR cells expressed CD40L, which can activate CD40+and other immune cells of the immune system, such as B cells, activated monocytes, DCs, and others.

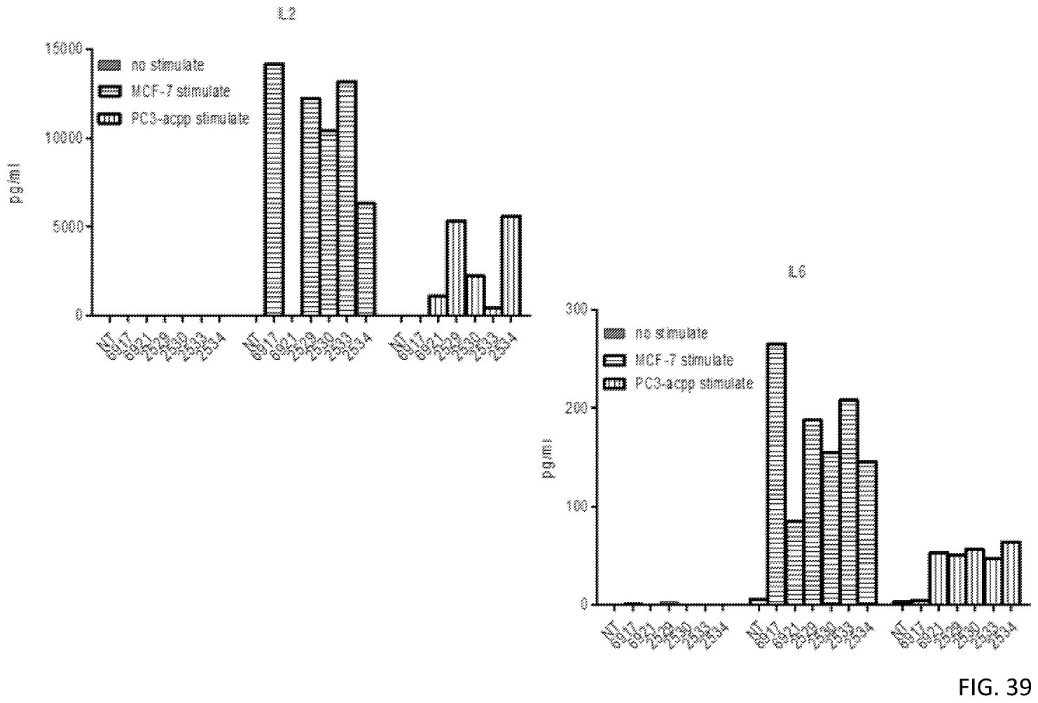

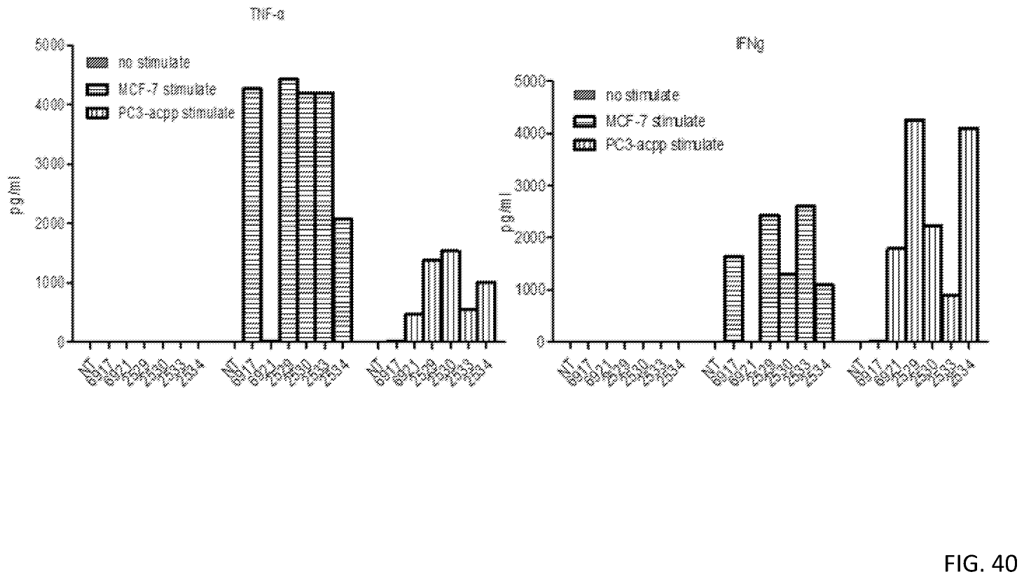

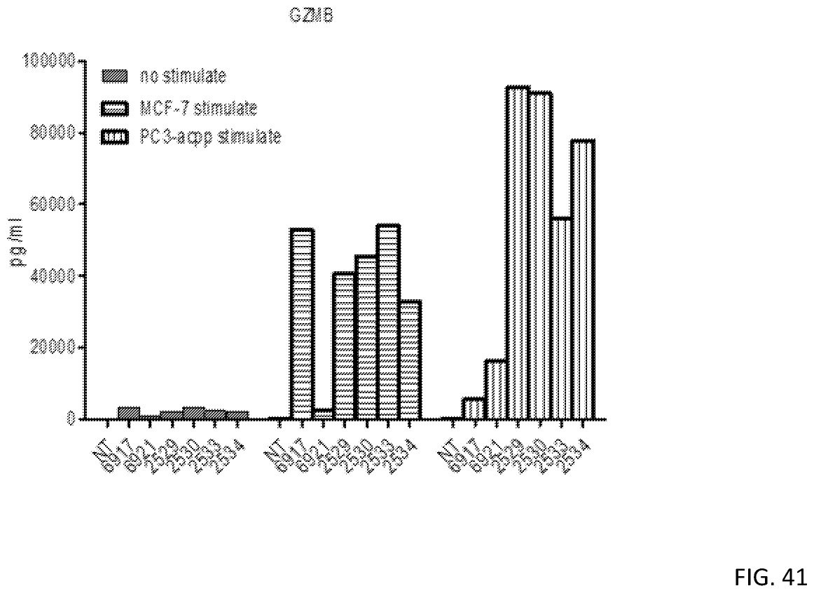

FIGS. 39-41 show cytokine release by CART cells and tanCAR T cells. NT, 6917, 6921, 6921, 2529, 2530, 2533, 2534, and substrate cells (MCF-7, PC3-acpp, 293T cells), were co-cultured for 24 hours and cytokine release was measured on Day 8.

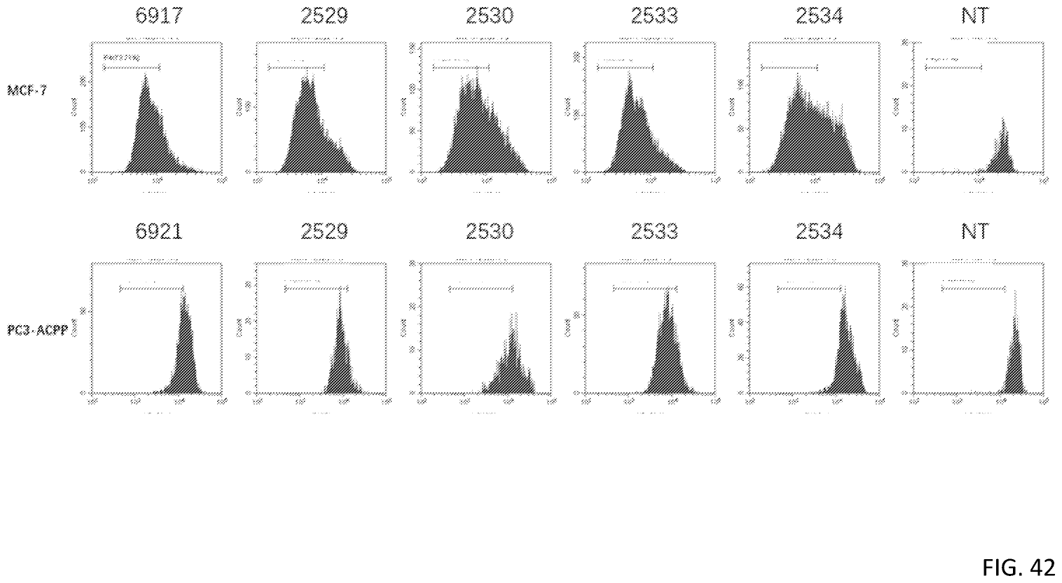

FIG. 42 shows the expansion of cells in each group after 5 days of stimulation with the corresponding substrate cells. As compared to control groups, tanCAR groups showed apparent expansion in response to both substrate cells. The proliferation of 6917, 6921, 2529, 2530, 2533, 2534, and NT was measured on Day 12 after co-culturing with substrate cells (MCF-7, PC3-acpp, 293T cells) for 5 days.

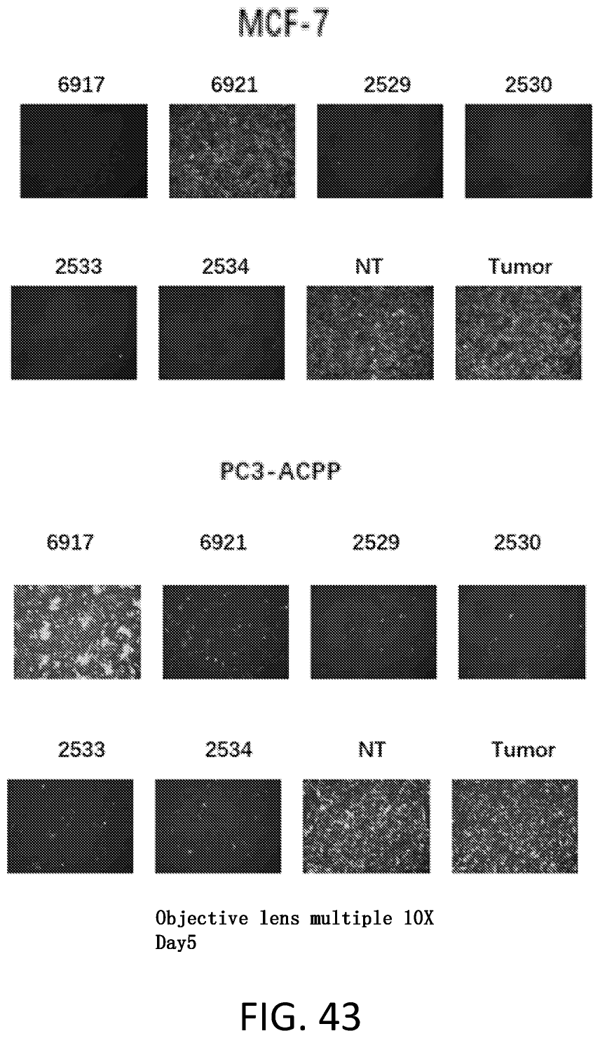

FIG. 43 shows killing assay results. The results indicate that 6917 inhibited MCF-7 and 6921 inhibited PC3-ACPP. The four groups of tanCAR T cells killed both substrate cells. NT was negative for the experiment. The control (Tumor) contained only tumor cells. The killing assay was performed for 6917, 6921, 2529, 2530, 2533, 2534, and NT cells after co-culturing with substrate cells for five days.

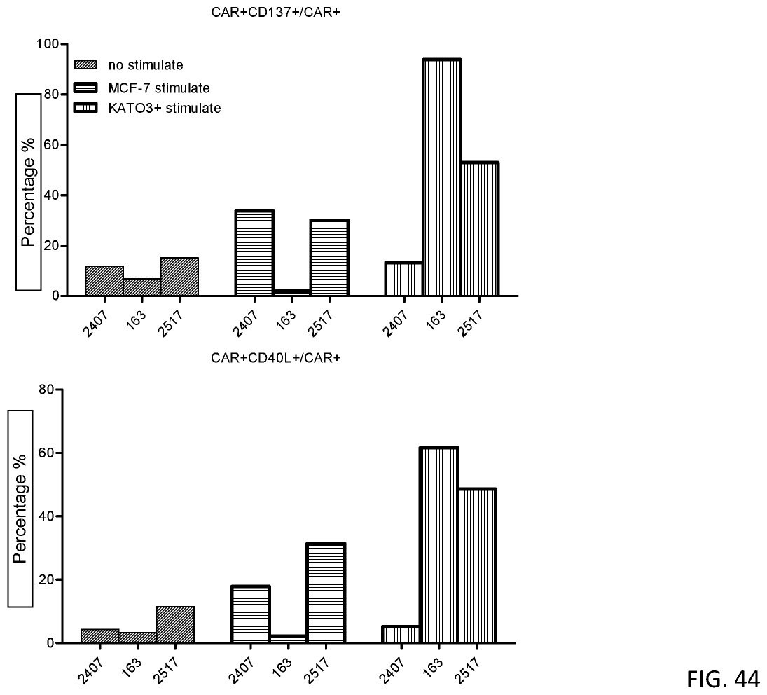

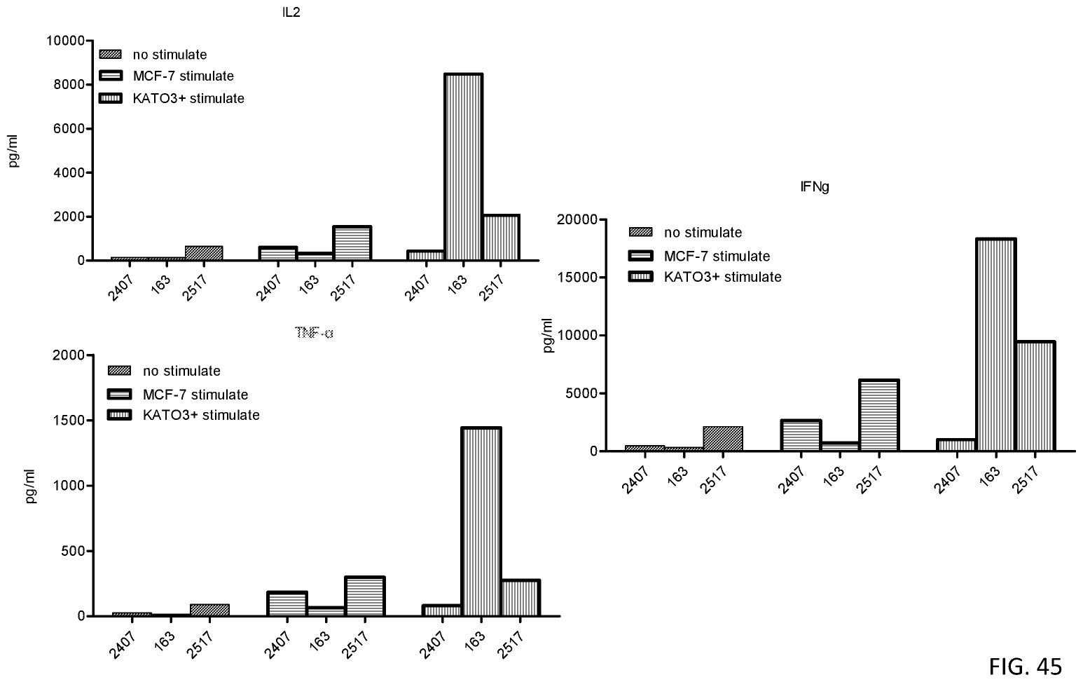

FIG. 44 shows expression of markers on CAR T cells and tanCAR T cells. FIG. 45 shows cytokine release using flow cytometry assay. On Day 8, 2407, 163, and 2517 were co-cultured with MCF-7, KATO3+, and 293T cells for 24 hours, and cytokine release assay was performed. TanCAR 2517 was activated by both MCF-7 and KATO3+substrate cells, and the expression of activation of Tan CAR 2517 were comparable to a cell with a single CAR. The corresponding CAR T cells were co-cultured with substrate cells (293T, MCF-7, and KATO3+) for 24 hrs, and the expression of CD40L was detected by flow cytometry.

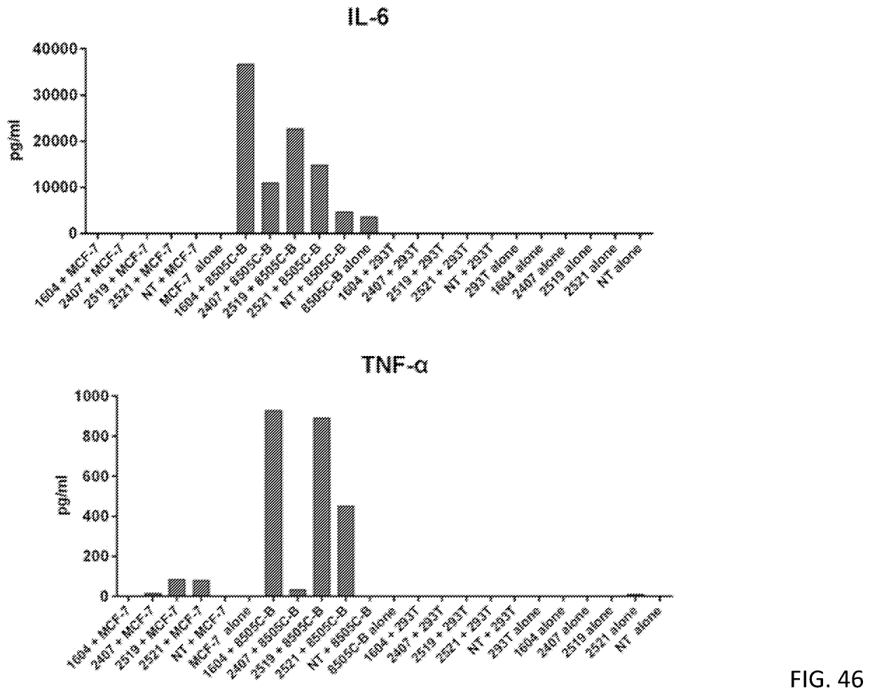

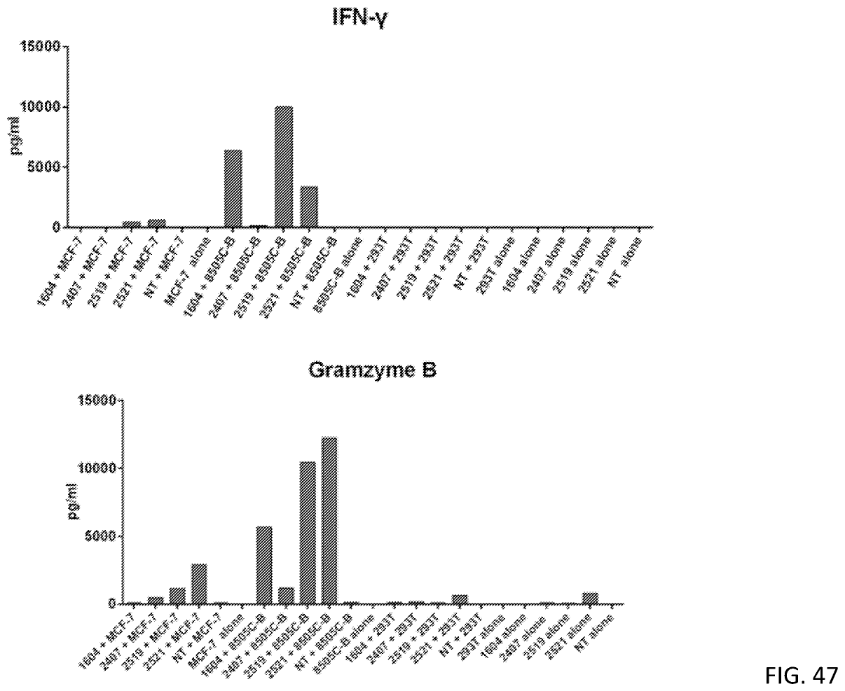

FIGS. 46 and 47 show cytokine release of various CART cells and tanCAR T cells in response to substrate cells. The experimental procedure are similar to those of the experiments described above.

TABLE 6

| SEQ | |
|---|---|
| ID NO: | Identity |
| 1 | SP |
| 2 | Hinge & transmembrane domain |
| 3 | Co-stimulatory region |
| 4 | CD3-zeta |
| 5 | scFV Humanized CD19 |
| 6 | scFV CD19 |
| 7 | scFv FZD10 |
| 8 | scFv TSHR |
| 9 | scFv PRLR |
| 10 | scFv Muc 17 |
| 11 | scFv GUCY2C |
| 12 | scFv CD207 |
| 13 | Prolactin (ligand) |
| 14 | scFv CD3 |
| 15 | scFv CD4 |
| 16 | scFv CD4-2 |
| 17 | scFv CD5 |
| 18 | WTCD3zeta |
| 19 | WTCD3zeta-BCMACAR full length |
| 20 | BCMACAR |
| 21 | MUC1CAR |
| 22 | m19CAR-IRES-MUC1CAR |
| 23 | hCD19CAR-IRES-MUC1CAR |
| 24 | hCD22CAR-IRES-MUC1CAR |
| 25 | BCMACAR-IRES-MUC1CAR |
| 26 | mCD19CAR-2A-MUC1CAR |
| 27 | hCD19CAR-2A-MUC1CAR |
| 28 | hCD22CAR-2A-MUC1CAR |
| 29 | BCMA-2A-MUC1CAR |
| 30 | Tumor associated MUC1 scFv 1 |
| 31 | Tumor associated MUC1 scFv-1 VH |
| 32 | Tumor associated MUC1 scFv-1 VL |
| 33 | Tumor associated MUC1 scFv 2 |
| 34 | Tumor associated MUC1 scFv2 VH |
| 35 | Tumor associated MUC1 scFv2 VL |
| 36 | CD40-CD28 (TM)-CD28 (IN) |
| 37 | CD40-CD28 (TM)-CD28 (IN) |
| 38 | OX40 extracellular |
| 39 | OX40 TM |
| 40 | OX40 intracellular |
| 41 | CD40 extracellular |
| 42 | CD40 TM |
| 43 | CD40 intracellular |
| 44 | 4-1BB extracellular |
| 45 | 4-1BB TM |
| 46 | 4-1BB intracellular |
| 47 | Her2 extracellular |
| 48 | Her2 TM |
| 49 | Her2 intracellular |
| 50 | GITR extracellular |
| 51 | GITR TM |
| 52 | GITR intracellular |
| 53 | CD28 extracellular |
| 54 | CD28TM |
| 55 | CD28 intracellular |
| 56 | ICOS extracellular |
| 57 | ICOS TM |
| 58 | ICOS intracellular |
| 59 | EGFR extracellular |
| 60 | EGFR TM |
| 61 | EFGR intracellular |
| 62 | CD27 extracellular |
| 63 | CD27 TM |
| 64 | CD27 intracellular |
| 65 | IL-18R1 cytoplasmic |
| 66 | IL-23R cytoplasmic |
| 67 | Gp130 (IL-6ST) cytoplasmic |
| 68 | IL-15Ra, cytoplasmic |
| 69 | Nucleic acid of SIRPα |
| 70 | Amino acid sequence of SIRPα |
| 71 | Modified Nucleic acid of SIRPα 1 |
| 72 | Modified Amino acid sequence of SIRPα 1 |
| 73 | Modified Nucleic acid of SIRPα 2 |
| 74 | Modified Amino acid sequence of SIRPα 2 |
| 75 | SIGLEC-10 WT (Sialic acid-binding Ig-like lectin 10 A |
| | receptor for CD24) |
| 76 | SIGLEC-10 (form2) |
| 77 | SIGLEC-10 (form3) |
| 78 | DN-SIGLEC-10 |
| 79 | DN-SIGLEC-10 (form2) |
| 80 | Hif VHL-interaction domain:Hif amino acid 344-417 |
| 81 | IL-12 & IFNγ |
| 82 | IFNγ & IL-12 |
| 83 | IL-12-VHL 1 |
| 84 | IL-12-VHL 2 |
| 85 | IFNγ-VHL1 |
| 86 | IFNγ-VHL2 |
| 87 | IL-6-VHL1 |
| 88 | IL-6-VHL2 |
| 89 | IL-6 & IFNγ |
| 90 | IFNγ & IL-6 |
| 91 | IL-12 & IFNγm (IFNγm:modified IFNγ) |
| 92 | IFNγm & IL-12 |
| 93 | IFNγm-VHL 1 |
| 94 | IFNγm-VHL2 |
| 95 | TNFα VHL 1 |
| 96 | TNFα VHL 2 |
| 97 | IL-12 & IL-6 |
| 98 | IL-6 & IL-12 |
| 99 | IL-6& IFNγm |
| 100 | IFNγm & IL-6 |
| 101 | Neo-2/15 |
| 102 | Neo-2/15-VHL 1 |
| 103 | Neo-2/15-VHL 2 |
| 104 | IFNβ-VHL 1 |
| 105 | IFNβ-VHL 2 |
| 106 | T2A (GSG residues are optional) |
| 107 | P2A(GSG residues are optional) |
| 108 | E2A(GSG residues are optional) |
| 109 | F2A(GSG residues are optional) |
| 110 | OX40-CD28(TM)-CD28(IN) |
| 111 | 4-1BB-CD28(TM)-CD28(IN) |
| 112 | Her2-CD28(TM)-CD28(IN) |
| 113 | GITR-CD28(TM)-CD28(IN) |
| 114 | ICOS-CD28(TM)-CD28(IN) |
| 115 | EGFR-CD28(TM)-CD28(IN) |
| 116 | CD27-CD28(TM)-CD28(IN) |
| 117 | HLA-A2-MAGEA4 substrate cell jurkat infection antigen |
| | sequence aa |
| 118 | HLA-A2-MAGEA4-41BB-CD3z CAR |
| 119 | HLA-A2-MAGEA4)-CD3 double-head antibody |
| | sequence/Signal peptide -- HLA-A2-MAGEA4-Fc(knob) |
| 120 | HLA-A2-MAGEA4)-CD3 double-head antibody |
| | sequence/Signal peptide -CD3-Fc(hole) |
| 121 | (HLA-A2-MAGEA4)-CD3 BITE sequence |
| 122 | type 1 diabetes/CAR sequence of HLA-A2-DRiPs 1BB-CD3z |
| 123 | (HLA-A2-DRiPs)-CD3 double-head antibody sequence/Signal |
| | peptide --HLA-A2-DRiPs-Fc(knob) |
| 124 | HLA-A2-DRiPs)-CD3 double-head antibody sequence/Signal |
| | peptide -CD3 -Fc(hole) |
| 125 | (HLA-A2-DRiPs) CD3 BITE sequences |
| 126 | Type 1 Diabetes: ADHPC -1 |
| 127 | Type 1 Diabetes: ADHPC - 2 |
| 128 | TanCAR CLDN 18.2 and GUCY2C 1 |
| 129 | TanCAR CLDN 18.2 and GUCY2C 2 |
| 130 | CLDN 18.2 scFv |
| 131 | Linker: (GGGGS)₃ |

All publications, patents and patent applications cited in this specification are incorporated herein by reference in their entireties as if each individual publication, patent or patent application were specifically and individually indicated to be incorporated by reference. While the foregoing has been described in terms of various embodiments, the skilled artisan will appreciate that various modifications, substitutions, omissions, and changes may be made without departing from the spirit thereof.

1. A method of enhancing T cell response, the method comprising:
introducing a nucleic acid encoding a chimeric antigen receptor (CAR) and a nucleic acid encoding an extracellular domain of CD40 into T cells to obtain modified T cells; and
contacting the modified T cells with an antigen that the CAR binds and a CD40 activator, thereby inducing a T cell response, wherein the T cell response is greater than the T cell response induced by contacting the modified T cells with the antigen without the CD40 activator.

2. The method of claim 1, wherein the CD40 activator is a CD40 ligand or a CD40 agonist.

3. The method of claim 1, wherein the nucleic acid encoding the extracellular domain of CD40 comprises nucleic acid sequence SEQ ID NO: 36.

4. The method of claim 1, wherein the T cell response comprises T cell expansion and/or T cell activation.

5. The method of claim 1, wherein the CAR comprises an antigen binding domain, a transmembrane domain, and an intracellular signaling domain.

6. The method of claim 5, wherein the antigen binding domain binds a tumor antigen, the tumor antigen comprising TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGSS, HMWMAA, o-acetyl-GD2, Folate receptor beta, TEM1/CD248, TEM7R, CLDN6, GPRCSD, CXORF61, CD97, CD179a, ALK, Polysialic acid, PLAC1, GloboH, NY-BR-1, UPK2, HAVCR1, ADRB3, PANX3, GPR20, LY6K, OR51 E2, TARP, W1-1, NY-ESO-1, LAGE-1a, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAXS, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRLS, or IGLL1.

7. The method of claim 5, wherein the intracellular signaling domain comprises a co-stimulatory signaling domain, or a primary signaling domain and a co-stimulatory signaling domain, and wherein the co-stimulatory signaling domain comprises a functional signaling domain of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, or NKG2D.

8. The method of claim 5, wherein the intracellular domain comprises a CD3 zeta signaling domain.

9. The method of claim 1, wherein the nucleic acid is an mRNA, which is not integrated into the genome of the modified cell.

10. The method of claim 1, wherein the nucleic acid encoding the extracellular domain of CD40 is associated with an oxygen-sensitive polypeptide domain.

11. The method of claim 10, wherein the oxygen-sensitive polypeptide domain comprises Hif1α VHL binding domain.

12. The method of claim 1, wherein the nucleic acid is regulated by a promoter comprising a binding site for a transcription modulator that modulates the expression and/or secretion of the therapeutic agent in the cell.

13. The method of claim 12, wherein the transcription modulator comprises one or more of Hif1a, NFAT, FOXP3, and NFkB.

14. The method of claim 1, wherein the modified T cells comprise a nucleic acid encoding a dominant negative form of an inhibitory immune checkpoint molecule or a receptor thereof.

15. The method of claim 14, wherein the inhibitory immune checkpoint molecule comprises programmed death 1 (PD-1), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIRD, natural killer cell receptor 2B4 (2B4), or CD160.

16. The method of claim 1, wherein the modified T cells have a reduced expression of endogenous TRAC gene.

17. The method of claim 1, wherein the modified T cells comprise a nucleic acid encoding hTERT or a nucleic acid encoding SV40LT, or a combination thereof.

18. The method of claim 1, wherein the modified T cells comprise an exogenous nucleic acid encoding a therapeutic agent, and the therapeutic agent comprises one or more of IL-1P, IL-2, IL-4, IL-5, IL-6, IL-7, IL-8, IL-10, IL-12, IL-13, IL-15, IL-17, IL-1Ra, IL-2R, IFNγ, MIP-In, MIP-IP, MCP-1, TNFα, GM-CSF, GCSF, CXCL9, CXCL10, CXCR factors, VEGF, RANTES, EOTAXIN, EGF, HGF, FGF-P, and ferritin.

19. The method of claim 1, wherein the modified T cells comprise at least one of 1) modified T cells comprising a first CAR binding a surface molecule of a white blood cell and a second CAR binding a solid tumor antigen, 2) modified T cells comprising the first CAR without the second CAR, and 3) modified T cell comprising the second CAR without the first CAR.

20. The method of claim 1, wherein the T cells are derived from a subject having cancer or a healthy donor.
